(19) United States
(12) Patent Application Publication
(10) Pub. No.: US 2022/0386609 A1
(43) Pub. Date: Dec. 8, 2022

(54) META-DIAMIDE INSECTICIDES

(71) Applicant: FMC Corporation, Philadelphia, PA (US)

(72) Inventors: George Philip LAHM, Wilmington, DE (US); Benjamin Kenneth SMITH, Newark, DE (US); Jeffrey Keith LONG, Wilmington, DE (US)

(21) Appl. No.: 17/761,486

(22) PCT Filed: Sep. 20, 2020
(86) PCT No.: PCT/US20/51701

§ 371 Date: Mar. 17, 2022

Related U.S. Application Data
(60) Provisional application No. 62/903,165, filed on Sep. 20, 2019.

(51) Int. Cl. A01N 43/58; A01N 43/54; A01P 7/04; C07C 233/11; C07D 237/24; C07C 233/65; A01N 37/18

(52) U.S. Cl. CPC A01N 43/58; A01N 43/54; A01P 7/04; C07C 233/11; C07D 237/24; C07C 233/65; A01N 37/18

(57) ABSTRACT

Disclosed are compounds of Formula 1, including all geometric and stereoisomers, N-oxides, and salts thereof, wherein Q, Y, R^{1a}, R^{1b}, Z, W, R², R³, R⁴, m, R^{5a} and R^{5b} are as defined in the disclosure. Also disclosed are compositions containing the compounds of Formula 1 and methods for controlling an invertebrate pest comprising contacting the invertebrate pest or its environment with a biologically effective amount of a compound or a composition of the invention.

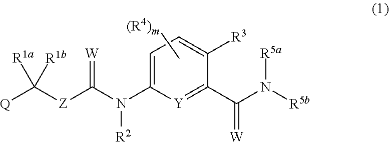

### CROSS-REFERENCE TO RELATED APPLICATION

This application claims the benefit of U.S. Provisional Application No. 62/903,165 filed Sep. 20, 2019.

### FIELD OF THE INVENTION

This invention relates to certain substituted meta-diamide compounds, their N-oxides, salts and compositions suitable for agronomic and nonagronomic uses, and methods of their use for controlling invertebrate pests such as arthropods in both agronomic and nonagronomic environments.

### BACKGROUND OF THE INVENTION

The control of invertebrate pests is extremely important in achieving high crop efficiency. Damage by invertebrate pests to growing and stored agronomic crops can cause significant reduction in productivity and thereby result in increased costs to the consumer. The control of invertebrate pests in forestry, greenhouse crops, ornamentals, nursery crops, stored food and fiber products, livestock, household, turf, wood products, and public and animal health is also important. Many products are commercially available for these purposes, but the need continues for new compounds that are more effective, less costly, less toxic, environmentally safer or have different sites of action.

PCT Patent Publications WO 2018/071327, WO 2016/168056, WO 2016/168058 and WO 2016/168059 disclose meta-diamide compounds and their use as insecticides in agricultural environments.

### SUMMARY OF THE INVENTION

This invention is directed to compounds of Formula 1 (including all stereoisomers), N-oxides, and salts thereof, compositions containing them and their use as insecticides:

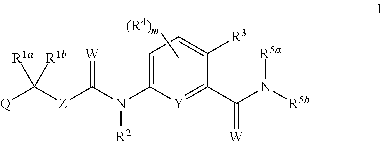

wherein
Q is a phenyl ring or a naphthalenyl ring system, each ring or ring system optionally substituted with up to 5 substituents independently selected from R⁶; or a 5- to 6-membered heterocyclic ring or an 8- to 11-membered heteroaromatic bicyclic ring system, each ring or ring system containing ring members selected from carbon atoms and 1 to 4 heteroatoms independently selected from up to 2 O, up to 2 S and up to 4 N atoms, wherein up to 2 ring members are independently selected from C(═O), C(═S), S(═O) and S(═O)₂, each ring or ring system optionally substituted with up to 5 substituents independently selected from R⁶;
Y is CR⁴ or N;
R^{1a} is CF₃, CHF₂, CCl₃, CHCl₂, CF₂Cl, CFCl₂ or CHFCl;
R^{1b} is H, halogen, hydroxy, C₁-C₃ alkyl, C₁-C₃ haloalkyl, C₁-C₃ alkoxy or C₁-C₃ haloalkoxy;
Z is CR^{7a}R^{7b}, NR^{7c}, O or S;
each W is independently O or S;
R² is H; or C₁-C₆ alkyl, C₂-C₆ alkenyl, C₂-C₆ alkynyl, C₂-C₆ alkylcarbonyl, C₂-C₆ alkoxycarbonyl or C₁-C₆ alkylsulfonyl, each optionally substituted with up to 5 substituents independently selected from halogen, cyano, hydroxy, C₁-C₃ alkyl, C₁-C₃ haloalkyl, C₁-C₃ alkoxy and C₁-C₃ haloalkoxy;
R³ is H, halogen, cyano, nitro, C₁-C₆ alkyl, C₁-C₆ haloalkyl, OR⁸ or S(═O)ₜR⁸; each R⁴ is independently halogen, cyano, nitro, C₁-C₆ alkyl, C₁-C₆ haloalkyl, OR⁸ or S(═O)ₜR⁸;
m is 0, 1, 2, or 3;
each t is independently 0, 1 or 2;
R^{5a} is H, cyano, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₂-C₆ alkoxyalkyl, C₂-C₆ haloalkoxyalkyl, C₁-C₆ alkylsulfonyl, C₁-C₆ haloalkylsulfonyl, C₂-C₆ alkylthioalkyl, C₂-C₆ alkylsulfinylalkyl, C₂-C₆ alkylsulfonylalkyl, C₂-C₆ alkylcarbonyl, C₂-C₆ haloalkylcarbonyl, C₂-C₆ alkoxycarbonyl, C₂-C₆ haloalkoxycarbonyl, C₂-C₆ alkylaminocarbonyl, C₃-C₆ dialkylaminocarbonyl, C₃-C₆ alkylaminocarbonylalkyl or C₃-C₆ haloalkylaminocarbonylalkyl;
R^{5b} is H, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₁-C₆ hydroxyalkyl, C₂-C₆ cyanoalkyl, C₃-C₆ cycloalkyl, C₃-C₆ halocycloalkyl, C₃-C₆ cycloalkenyl, C₃-C₆ halocycloalkenyl, C₄-C₁₀ alkylcycloalkyl, C₄-C₁₀ haloalkylcycloalkyl, C₄-C₁₀ cycloalkylalkyl, C₄-C₁₀ halocycloalkylalkyl, C₅-C₁₀ alkylcycloalkylalkyl, C₂-C₆ alkoxyalkyl, C₂-C₆ haloalkoxyalkyl, C₄-C₁₀ cycloalkoxyalkyl, C₃-C₆ alkoxyalkoxyalkyl, C₂-C₆ alkylthioalkyl, C₂-C₆ haloalkylthioalkyl, C₂-C₆ alkylsulfinylalkyl, C₂-C₆ haloalkylsulfinylalkyl, C₂-C₆ alkylsulfonylalkyl, C₂-C₆ haloalkylsulfonylalkyl, C₂-C₆ alkylaminoalkyl, C₂-C₆ haloalkylaminoalkyl, C₃-C₈ dialkylaminoalkyl, C₄-C₁₀ cycloalkylaminoalkyl; or
R^{5b} is a phenyl ring or a naphthalenyl ring system, each ring or ring system optionally substituted with up to 5 substituents independently selected from R⁹; or a 5- to 6-membered heterocyclic ring or an 8- to 11-membered heteroaromatic bicyclic ring system, each ring or ring system containing ring members selected from carbon atoms and 1 to 4 heteroatoms independently selected from up to 2 O, up to 2 S and up to 4 N atoms, wherein up to 2 ring members are independently selected from C(═O), C(═S), S(═O) and S(═O)₂, each ring optionally substituted with up to 5 substituents independently selected from R⁹; or
R^{5b} is -A(CR^{10a}R^{10b})ₙB or NR^{21a}R^{21b}; or
R^{5a} and R^{5b} are taken together to form a 4- to 6-membered fully saturated heterocyclic ring, each ring containing ring members, in addition to the connecting nitrogen atom, selected from carbon atoms and up to 2 heteroatoms independently selected from up to 2 O, up to 2 S and up to 2 N atoms, each ring optionally substituted with up to 3 substituents independently selected from R¹³;
A is a direct bond, O or NR¹¹;
n is 0, 1, 2 or 3, provided that when n is 0, then A is O or NR¹¹;
B is a phenyl ring optionally substituted with up to 5 substituents independently selected from R¹²; or a 4- to 7-membered heterocyclic ring containing ring members selected from carbon atoms and 1 to 4 heteroatoms independently selected from up to 2 O, up to 2 S and up to 4 N atoms, wherein up to 3 carbon atom ring members are independently selected from C(═O) and C(═S), the ring optionally substituted with up to 3 substituents independently selected from R¹²;
each R⁶ is independently cyano, halogen, hydroxy, nitro, C(═O)OH, NR^{14a}R^{14b}, C(═O)NR^{14a}R^{14b}, C(═S)NR^{14a}R^{14b} or —U—V-T; or C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₃-C₆ cycloalkyl, C₃-C₆ halocycloalkyl, C₂-C₆ cyanoalkyl, C₁-C₆ hydroxyalkyl, C₄-C₁₀ alkylcycloalkyl, C₄-C₁₀ cycloalkylalkyl, C₃-C₆ cycloalkenyl, C₃-C₆ halocycloalkenyl, C₂-C₆ alkoxyalkyl, C₄-C₁₀ cycloalkoxyalkyl, C₃-C₁₀ alkoxyalkoxyalkyl, C₂-C₆ alkylthioalkyl, C₂-C₆ alkylsulfinylalkyl, C₁-C₆ alkoxy, C₁-C₆ haloalkoxy, C₃-C₆ cycloalkoxy, C₃-C₆ halocycloalkoxy, C₄-C₁₀ cycloalkylalkoxy, C₂-C₆ alkenyloxy, C₂-C₆ haloalkenyloxy, C₂-C₆ alkoxyalkoxy, C₂-C₆ alkylcarbonyloxy, C₁-C₆ alkylthio, C₁-C₆ haloalkylthio, C₃-C₆ cycloalkylthio, C₁-C₆ alkylsulfinyl, C₁-C₆ haloalkylsulfinyl, C₁-C₆ alkylsulfonyl, C₁-C₆ haloalkylsulfonyl, C₃-C₆ cycloalkylsulfonyl, C₁-C₆ alkylamino, C₂-C₆ dialkylamino, C₁-C₆ haloalkylamino, C₂-C₆ halodialkylamino or C₃-C₆ cycloalkylamino, each optionally substituted with up to 3 substituents independently selected from R¹⁵;
R^{7a} is H, C₁-C₆ alkyl, C₁-C₆ haloalkyl, C₃-C₆ cycloalkyl, C₄-C₆ cycloalkylalkyl and C₄-C₆ alkylcycloalkyl;
R^{7b} is H, C₁-C₆ alkyl, C₁-C₆ haloalkyl, C₂-C₆ cyanoalkyl, C₃-C₆ cycloalkyl, C₃-C₆ halocycloalkyl, C₄-C₆ alkylcycloalkyl, C₄-C₆ cycloalkylalkyl, C₄-C₆ halocycloalkylalkyl, C₂-C₆ alkoxyalkyl, C₂-C₆ haloalkoxyalkyl, C₄-C₆ cycloalkoxyalkyl, C₃-C₆ alkoxyalkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₂-C₆ haloalkylaminoalkyl or C₃-C₆ dialkylaminoalkyl, each optionally substituted with up to 1 substituent selected from cyano, hydroxy, nitro, C₂-C₄ alkylcarbonyl or C₂-C₄ alkoxycarbonyl; or phenyl optionally substituted with up to 3 substituents independently selected from R¹⁶;
R^{7c} is H, C(═O)H, C₁-C₄ alkyl, C₁-C₄ haloalkyl or C₂-C₄ alkylcarbonyl;
each R⁸ is independently H, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₃-C₆ cycloalkyl or C₃-C₆ halocycloalkyl; or phenyl or benzyl each ring optionally substituted with up to 4 substituents independently selected from R¹⁷;
each R⁹ is independently amino, cyano, halogen, hydroxy, nitro, C₁-C₆ alkyl, C₁-C₆ haloalkyl, C₃-C₆ cycloalkyl, C₃-C₆ halocycloalkyl, C₁-C₆ alkoxy, C₁-C₆ haloalkoxy, C₂-C₆ alkoxyalkoxy, C₁-C₆ alkylthio, C₁-C₆ alkylsulfinyl, C₁-C₆ alkylsulfonyl, C₁-C₆ haloalkylsulfonyl, C₂-C₆ alkylcarbonyl, C₂-C₆ haloalkylcarbonyl, C₂-C₆ alkoxycarbonyl, C₁-C₆ alkylamino, C₂-C₆ dialkylamino, C₂-C₆ alkylaminocarbonyl or C₃-C₆ dialkylaminocarbonyl;
each R^{10a} is independently H, halogen, cyano or C₁-C₄ alkyl;
each R^{10b} is independently H or C₁-C₄ alkyl;
R¹¹ is H, cyano, hydroxy, C₁-C₃ alkyl, C₁-C₃ haloalkyl, C₂-C₃ alkenyl, C₂-C₃ haloalkenyl, C₁-C₃ alkoxy, C₂-C₃ alkylcarbonyl or C₂-C₃ haloalkylcarbonyl; each R¹² is independently halogen, cyano, C₁-C₃ alkyl, C₁-C₃ haloalkyl, C₁-C₃ alkoxy or C₁-C₃ haloalkoxy;
each R¹³ is independently halogen, cyano, hydroxy, nitro, C₁-C₆ alkyl, C₁-C₆ haloalkyl, C₂-C₆ alkenyl, C₂-C₆ haloalkenyl, C₂-C₆ alkynyl C₂-C₆ haloalkynyl, C₃-C₆ cycloalkyl, C₃-C₆ halocycloalkyl, C₂-C₆ alkoxyalkyl, C₂-C₆ haloalkoxyalkyl, C₁-C₆ alkoxy, C₁-C₆ haloalkoxy, C₃-C₆ cycloalkoxy, C₃-C₆ halocycloalkoxy, C₂-C₆ alkenyloxy, C₂-C₆ haloalkenyloxy, C₂-C₆ alkynyloxy, C₂-C₆ haloalkynyloxy, C₂-C₆ alkoxyalkoxy, C₂-C₆ haloalkoxyalkoxy, C₁-C₆ alkylthio, C₁-C₆ haloalkylthio, C₂-C₆ alkylcarbonyl, C₂-C₆ haloalkylcarbonyl, C₂-C₆ alkoxycarbonyl, C₂-C₆ haloalkoxycarbonyl, C₂-C₆ alkylaminocarbonyl, C₃-C₆ dialkylaminocarbonyl, C₂-C₆ alkylcarbonyloxy, C₂-C₆ haloalkylcarbonyloxy, C₁-C₆ alkylamino, C₂-C₆ dialkylamino, C₁-C₆ haloalkylamino or C₂-C₆ halodialkylamino;
each R^{14a} is independently H, cyano, hydroxy, C₁-C₄ alkyl, C₁-C₄ haloalkyl, C₂-C₄ alkenyl, C₂-C₄ haloalkenyl, C₂-C₄ alkynyl, C₂-C₄ haloalkynyl, C₁-C₄ alkoxy, C₁-C₄ haloalkoxy, C₂-C₄ alkoxyalkyl, C₁-C₄ alkylsulfonyl, C₁-C₄ haloalkylsulfonyl, C₂-C₄ alkylthioalkyl, C₂-C₄ alkylsulfinylalkyl, C₂-C₄ alkylsulfonylalkyl, C₂-C₄ alkylcarbonyl, C₂-C₄ haloalkylcarbonyl, C₄-C₆ cycloalkylcarbonyl, C₂-C₄ alkoxycarbonyl, C₃-C₅ alkoxycarbonylalkyl, C₂-C₄ alkylaminocarbonyl or C₃-C₅ dialkylaminocarbonyl;
each R^{14b} is independently H, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₁-C₆ hydroxyalkyl, C₂-C₆ cyanoalkyl, C₃-C₆ cycloalkyl, C₃-C₆ halocycloalkyl, C₃-C₆ cycloalkenyl, C₃-C₆ halocycloalkenyl, C₄-C₁₀ alkylcycloalkyl, C₄-C₁₀ cycloalkylalkyl, C₄-C₁₀ halocycloalkylalkyl, C₂-C₆ alkoxyalkyl, C₂-C₆ haloalkoxyalkyl, C₄-C₁₀ cycloalkoxyalkyl, C₃-C₆ alkoxyalkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₂-C₆ haloalkylaminoalkyl, C₃-C₆ dialkylaminoalkyl or C₄-C₁₀ cycloalkylaminoalkyl; each R¹⁵ is independently amino, cyano, halogen, hydroxy, nitro, C₁-C₄ alkyl, C₁-C₄ haloalkyl, C₃-C₆ cycloalkyl, C₃-C₆ halocycloalkyl, C₁-C₄ alkoxy, C₁-C₄ haloalkoxy, C₂-C₄ alkoxyalkoxy, C₁-C₄ alkylthio, C₁-C₄ alkylsulfinyl, C₁-C₄ alkylsulfonyl, C₁-C₄ haloalkylsulfonyl, C₂-C₄ alkylcarbonyl, C₂-C₄ haloalkylcarbonyl, C₂-C₅ alkoxycarbonyl, C₁-C₄ alkylamino or C₂-C₅ dialkylamino;
each U is independently a direct bond, C(═O)O, C(═O)N(R¹⁸) or C(═S)N(R¹⁹), wherein the atom to the left is connected to Q, and the atom to the right is connected to V;
each V is independently a direct bond; or C₁-C₆ alkylene, C₂-C₆ alkenylene, C₃-C₆ alkynylene, each optionally substituted with up to 3 substituents independently selected from halogen, cyano, nitro, hydroxy, C₁-C₂ alkyl, C₁-C₂ haloalkyl, C₁-C₂ alkoxy and C₁-C₂ haloalkoxy;
each T is independently phenyl or phenoxy, each optionally substituted with up to 3 substituents independently selected from R²⁰; or
each T is independently a 5- to 6-membered heteroaromatic ring, each ring containing ring members selected from carbon atoms and 1 to 4 heteroatoms independently selected from up to 2 O, up to 2 S and up to 4 N atoms, each ring optionally substituted with up to 3 substituents independently selected from R²⁰; or
each T is independently a 3- to 7-membered nonaromatic heterocyclic ring containing ring members selected from carbon atoms and 1 to 4 heteroatoms independently selected from up to 2 O, up to 2 S and up to 4 N atoms, wherein up to 2 ring members are independently selected from C(═O), C(═S), S(═O) and S(═O)₂, each ring optionally substituted with up to 3 substituents independently selected from R²⁰;
each R¹⁶ is independently halogen, cyano, nitro, C₁-C₆ alkyl, C₁-C₆ haloalkyl, C₁-C₆ alkoxy, C₁-C₆ haloalkoxy, C₁-C₆ alkylthio, C₁-C₆ haloalkylthio C₁-C₆ alkylsulfinyl, C₁-C₆ haloalkylsulfinyl, C₁-C₆ alkylsulfonyl or C₁-C₆ haloalkylsulfonyl;
each R¹⁷ is independently halogen, cyano, nitro, C₁-C₆ alkyl, C₁-C₆ haloalkyl, C₁-C₆ alkoxy, C₁-C₆ haloalkoxy, C₁-C₆ alkylthio, C₁-C₆ haloalkylthio C₁-C₆ alkylsulfinyl, C₁-C₆ haloalkylsulfinyl, C₁-C₆ alkylsulfonyl or C₁-C₆ haloalkylsulfonyl;
each R¹⁸ and R¹⁹ is independently H, cyano, hydroxy, C₁-C₄ alkyl, C₁-C₄ haloalkyl, C₂-C₄ alkylcarbonyl, C₂-C₄ haloalkylcarbonyl, C₂-C₄ alkoxycarbonyl or C₂-C₄ haloalkoxycarbonyl;
each R²⁰ is independently halogen, cyano, hydroxy, nitro, C₁-C₄ alkyl, C₁-C₄ haloalkyl, C₂-C₄ alkenyl, C₁-C₄ alkoxy, C₂-C₄ alkylcarbonyl or C₂-C₄ alkoxycarbonyl;
R^{21a} is H, C₁-C₄ alkyl, C₁-C₄ haloalkyl or C₂-C₄ alkylcarbonyl; and
R^{21b} is H, cyano, C₁-C₅ alkyl, C₁-C₅ haloalkyl, C₁-C₆ cycloalkyl, C₁-C₆ halocycloalkyl, C₂-C₅ alkylcarbonyl, C₂-C₅ haloalkylcarbonyl, C₄-C₇ cycloalkylcarbonyl, C₂-C₅ alkoxycarbonyl, C₂-C₅ haloalkoxycarbonyl, C₃-C₅ alkoxycarbonylalkyl, C₂-C₅ alkylaminocarbonyl or C₃-C₅ dialkylaminocarbonyl; or
R^{21a} and R^{21b} are taken together to form a 5- to 6-membered fully saturated heterocyclic ring, each ring containing ring members, in addition to the connecting nitrogen atom, selected from carbon atoms and up to 2 heteroatoms independently selected from up to 2 O, up to 2 S and up to 2 N atoms, each ring optionally substituted with up to 5 substituents independently selected from halogen, cyano, nitro, hydroxy, C₁-C₃ alkyl, C₁-C₃ haloalkyl, C₁-C₃ alkoxy and C₁-C₃ haloalkoxy.

This invention also provides a composition comprising a compound of Formula 1, an N-oxide or a salt thereof, and at least one additional component selected from the group consisting of surfactants, solid diluents and liquid diluents. In one embodiment, this invention also provides a composition for controlling an invertebrate pest comprising a compound of Formula 1, an N-oxide or a salt thereof, and at least one additional component selected from the group consisting of surfactants, solid diluents and liquid diluents, said composition optionally further comprising at least one additional biologically active compound or agent (e.g., fungicide).

This invention further provides a spray composition for controlling an invertebrate pest comprising a compound of Formula 1, an N-oxide or a salt thereof, or the compositions described above, and a propellant. This invention also provides a bait composition for controlling an invertebrate pest comprising a compound of Formula 1, an N-oxide or a salt thereof, or the compositions described in the embodiments above, one or more food materials, optionally an attractant, and optionally a humectant.

This invention further provides a trap device for controlling an invertebrate pest comprising said bait composition and a housing adapted to receive said bait composition, wherein the housing has at least one opening sized to permit the invertebrate pest to pass through the opening so the invertebrate pest can gain access to said bait composition from a location outside the housing, and wherein the housing is further adapted to be placed in or near a locus of potential or known activity for the invertebrate pest.

This invention provides a method for controlling an invertebrate pest comprising contacting the invertebrate pest or its environment with a biologically effective amount of a compound of Formula 1, an N-oxide or a salt thereof, (e.g., as a composition described herein). This invention also relates to such method wherein the invertebrate pest or its environment is contacted with a composition comprising a biologically effective amount of a compound of Formula 1, an N-oxide or a salt thereof, and at least one additional component selected from the group consisting of surfactants, solid diluents and liquid diluents, said composition optionally further comprising a biologically effective amount of at least one additional biologically active compound or agent.

This invention also provides a method for protecting a seed from an invertebrate pest comprising contacting the seed with a biologically effective amount of a compound of Formula 1, an N-oxide or a salt thereof, (e.g., as a composition described herein). This invention also relates to the treated seed. This invention further provides a method for protecting an animal from an invertebrate parasitic pest comprising administering to the animal a parasiticidally effective amount of a compound of Formula 1, an N-oxide or a salt thereof, (e.g., as a composition described herein). This invention also provides for the use of a compound of Formula 1, an N-oxide or a salt thereof, (e.g., as a composition described herein) in protecting an animal from an invertebrate pest.

This invention also provides a method for increasing vigor of a crop plant comprising contacting the crop plant, the seed from which the crop plant is grown or the locus (e.g., growth medium) of the crop plant with a biologically effective amount of a compound of Formula 1 (e.g., as a composition described herein).

The invention also relates to compounds of Formula 2 (including all stereoisomers), N-oxides, and salts thereof:

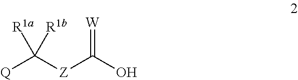

wherein
Q, R^{1a}, R^{1b}, Z and W are as defined above for Formula 1.

### DETAILS OF THE INVENTION

As used herein, the terms “comprises,” “comprising,” “includes,” “including,” “has,” “having,” “contains,” “containing,” “characterized by” or any other variation thereof, are intended to cover a non-exclusive inclusion, subject to any limitation explicitly indicated. For example, a composition, mixture, process, method, article, or apparatus that comprises a list of elements is not necessarily limited to only those elements but may include other elements not expressly listed or inherent to such composition, mixture, process, method, article, or apparatus.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition, method or apparatus that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in this disclosure, the term “invertebrate pest” includes arthropods, gastropods, nematodes and helminths of economic importance as pests. The term “arthropod” includes insects, mites, spiders, scorpions, centipedes, millipedes, pill bugs and symphylans. The term “gastropod” includes snails, slugs and other Stylommatophora. The term “nematode” includes members of the phylum Nematoda, such as phytophagous nematodes and helminth nematodes parasitizing animals. The term “helminth” includes all of the parasitic worms, such as roundworms (phylum Nematoda), heartworms (phylum Nematoda, class Secernentea), flukes (phylum Platyhelminthes, class Tematoda), acanthocephalans (phylum Acanthocephala), and tapeworms (phylum Platyhelminthes, class Cestoda).

In the context of this disclosure “invertebrate pest control” means inhibition of invertebrate pest development (including mortality, feeding reduction, and/or mating disruption), and related expressions are defined analogously.

The term “agronomic” refers to the production of field crops such as for food and fiber and includes the growth of maize or corn, soybeans and other legumes, rice, cereal (e.g., wheat, oats, barley, rye and rice), leafy vegetables (e.g., lettuce, cabbage, and other cole crops), fruiting vegetables (e.g., tomatoes, pepper, eggplant, crucifers and cucurbits), potatoes, sweet potatoes, grapes, cotton, tree fruits (e.g., pome, stone and citrus), small fruit (e.g., berries and cherries) and other specialty crops (e.g., canola, sunflower and olives).

The term “nonagronomic” refers to other than field crops, such as horticultural crops (e.g., greenhouse, nursery or ornamental plants not grown in a field), residential, agricultural, commercial and industrial structures, turf (e.g., sod farm, pasture, golf course, lawn, sports field, etc.), wood products, stored product, agro-forestry and vegetation management, public health (i.e. human) and animal health (e.g., domesticated animals such as pets, livestock and poultry, undomesticated animals such as wildlife) applications.

The term “crop vigor” refers to rate of growth or biomass accumulation of a crop plant. An “increase in vigor” refers to an increase in growth or biomass accumulation in a crop plant relative to an untreated control crop plant. The term “crop yield” refers to the return on crop material, in terms of both quantity and quality, obtained after harvesting a crop plant. An “increase in crop yield” refers to an increase in crop yield relative to an untreated control crop plant.

The term “biologically effective amount” refers to the amount of a biologically active compound (e.g., a compound of Formula 1) sufficient to produce the desired biological effect when applied to (i.e. contacted with) an invertebrate pest to be controlled or its environment, or to a plant, the seed from which the plant is grown, or the locus of the plant (e.g., growth medium) to protect the plant from injury by the invertebrate pest or for other desired effect (e.g., increasing plant vigor).

Generally when a molecular fragment (i.e. radical) is denoted by a series of atom symbols (e.g., C, H, N, O and S) the implicit point or points of attachment will be easily recognized by those skilled in the art. In some instances herein, particularly when alternative points of attachment are possible, the point or points of attachment may be explicitly indicated by a hyphen (“-”). For example, “—NCS” indicates that the point of attachment is the nitrogen atom (i.e. isothiocyanato, not thiocyanato).

As used herein, the term “alkylating agent” refers to a chemical compound in which a carbon-containing radical is bound through a carbon atom to a leaving group such as halide or sulfonate, which is displaceable by bonding of a nucleophile to said carbon atom. Unless otherwise indicated, the term “alkylating” does not limit the carbon-containing radical to alkyl; the carbon-containing radicals in alkylating agents include the variety of carbon-bound substituent radicals specified, for example, for R⁶.

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” can also include moieties comprised of multiple triple bonds such as 2,5-hexadiynyl.

“Alkylene” denotes a straight-chain or branched alkanediyl. Examples of “alkylene” include CH₂, CH₂CH₂, CH(CH₃), CH₂CH₂CH₂, CH₂CH(CH₃), and the different butylene isomers. “Alkenylene” denotes a straight-chain or branched alkenediyl containing one olefinic bond. Examples of “alkenylene” include CH═CH, CH₂CH═CH, CH═C(CH₃) and the different butenylene isomers. “Alkynylene” denotes a straight-chain or branched alkynediyl containing one triple bond. Examples of “alkynylene” include CH₂C≡C, C≡CCH₂, and the different butynylene, pentynylene or hexynylene isomers.

“Alkoxy” includes, for example, methoxy, ethoxy, n-propyloxy, i-propyloxy, and the different butoxy, pentoxy and hexyloxy isomers. “Alkenyloxy” includes straight-chain and branched alkenyl attached to and linked through an oxygen atom. Examples of “alkenyloxy” include H₂C═CHCH₂O and CH₃CH═CHCH₂O. “Alkynyloxy” includes straight-chain and branched alkynyloxy moieties. Examples of “alkynyloxy” include HC≡CCH₂O and CH₃C≡CCH₂O.

The term “alkylthio” includes straight-chain and branched alkylthio moieties such as methylthio, ethylthio, and the different propylthio, butylthio, pentylthio and hexylthio isomers. “Alkylsulfinyl” includes both enantiomers of an alkylsulfinyl group. Examples of “alkylsulfinyl” include CH₃S(═O), CH₃CH₂S(═O), CH₃CH₂CH₂S(═O), (CH₃)₂CHS(═O), and the different butylsulfinyl, pentylsulfinyl and hexylsulfinyl isomers. Examples of “alkylsulfonyl” include CH₃S(═O)₂, CH₃CH₂S(═O)₂, CH₃CH₂CH₂S(═O)₂, (CH₃)₂CHS(═O)₂, and the different butylsulfonyl, pentylsulfonyl and hexylsulfonyl isomers.

“Alkylthioalkyl” denotes alkylthio substitution on alkyl. Examples of “alkylthioalkyl” include CH₃SCH₂, CH₃SCH₂CH₂, CH₃CH₂SCH₂, CH₃CH₂CH₂SCH₂ and CH₃CH₂SCH₂CH₂; “alkylsulfinylalkyl” and “alkylsulfonylalkyl” include the corresponding sulfoxides and sulfones, respectively.

“Alkylamino” includes an NH radical substituted with a straight-chain or branched alkyl group. Examples of “alkylamino” include CH₃CH₂NH, CH₃CH₂CH₂NH, and (CH₃)₂CHCH₂NH. Examples of “dialkylamino” include (CH₃)₂N, (CH₃CH₂CH₂)₂N and CH₃CH₂(CH₃)N. “Alkylaminoalkyl” denotes alkylamino substitution on alkyl. Examples of “alkylaminoalkyl” include CH₃NHCH₂, CH₃NHCH₂CH₂, CH₃CH₂NHCH₂, CH₃CH₂CH₂CH₂NHCH₂ and CH₃CH₂NHCH₂CH₂.

“Alkylcarbonyl” denotes a straight-chain or branched alkyl group bonded to a C(═O) moiety. Examples of “alkylcarbonyl” include CH₃C(═O), CH₃CH₂CH₂C(═O) and (CH₃)₂CHC(═O). Examples of “alkoxycarbonyl” include CH₃OC(═O), CH₃CH₂OC(═O), CH₃CH₂CH₂OC(═O), (CH₃)₂CHOC(═O), and the different butoxy-, pentoxy- and hexyloxycarbonyl isomers. Examples of “alkylaminocarbonyl” include CH₃NHC(═O), CH₃CH₂NHC(═O), CH₃CH₂CH₂NHC(═O), (CH₃)₂CHNHC(═O), and the different butylamino-, pentylamino- and hexylaminocarbonyl isomers. Examples of “dialkyl-aminocarbonyl” include (CH₃)₂NC(═O), (CH₃CH₂)₂NC(═O), CH₃CH₂(CH₃)NC(═O), (CH₃)₂CH(CH₃)NC(═O) and CH₃CH₂CH₂(CH₃)NC(═O).

“Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂OCH₂ and CH₃CH₂OCH₂CH₂.

“Alkoxyalkoxy” denotes alkoxy substitution on another alkoxy moiety. “Alkoxyalkoxyalkyl” denotes alkoxyalkoxy substitution on alkyl. Examples of “alkoxyalkoxyalkyl” include CH₃OCH₂OCH₂ CH₃OCH₂OCH₂CH₂ and CH₃CH₂OCH₂OCH₂. “Alkylthioalkyl” denotes alkylthio substitution on alkyl. Examples of “alkylthioalkyl” include CH₃SCH₂, CH₃SCH₂CH₂, CH₃CH₂SCH₂, CH₃CH₂CH₂SCH₂ and CH₃CH₂SCH₂CH₂; “alkylsulfinylalkyl” and “alkylsulfonylalkyl” include the corresponding sulfoxides and sulfones, respectively.

The term “alkylcarbonyloxy” denotes a straight-chain or branched alkyl bonded to a C(═O)O moiety. Examples of “alkylcarbonyloxy” include CH₃CH₂C(═O)O and (CH₃)₂CHC(═O)O. The term “alkoxycarbonylalkyl” denotes alkoxycarbonyl substitution on alkyl. Examples of “alkoxycarbonylalkyl” include CH₃CH₂OC(═O)CH₂, (CH₃)₂CHOC(═O)CH₂ and CH₃OC(═O)CH₂CH₂. The term “alkylaminocarbonylalkyl” denotes a straight-chain or branched alkylaminocarbonyl attached to alkyl. Examples of “alkylaminocarbonylalkyl” include (CH₃)₂CHCH₂NHC(═O)CH₂ and CH₃CH₂NHC(═O)CH₂.

“Cycloalkyl” includes, for example, cyclopropyl, cyclobutyl, cyclopentyl and cyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to a straight-chain or branched alkyl group. The term “alkylcycloalkyl” denotes alkyl substitution on a cycloalkyl moiety and includes, for example, ethylcyclopropyl, i-propylcyclobutyl, methylcyclopentyl and methylcyclohexyl. “Alkylcycloalkylalkyl” denotes alkylcycloalkyl substitution on alkyl. Examples of “alkylcycloalkylalkyl” include methylcyclohexylmethyl and ethylcycloproylmethyl. “Cycloalkenyl” includes groups such as cyclopentenyl and cyclohexenyl as well as groups with more than one double bond such as 1,3- or 1,4-cyclohexadienyl.

The term “cycloalkoxy” denotes cycloalkyl attached to and linked through an oxygen atom including, for example, cyclopentyloxy and cyclohexyloxy. The term “cycloalkoxyalkyl” denotes cycloalkoxy substitution on an alkyl moiety. Examples of “cycloalkoxyalkyl” include cyclopropyloxymethyl, cyclopentyloxyethyl, and other cycloalkoxy groups bonded to a straight-chain or branched alkyl moiety. “Cycloalkylcarbonyl” denotes cycloalkyl bonded to a C(═O) group including, for example, cyclopropylcarbonyl and cyclopentylcarbonyl. The term “cycloalkylthio” denotes cycloalkyl attached to and linked through a sulfur atom such as cyclopropylthio and cyclopentylthio; “cycloalkylsulfonyl” includes the corresponding sulfones. “Cycloalkylamino” denotes an NH radical substituted with cycloalkyl. Examples of “cycloalkylamino” include cyclopropylamino and cyclohexylamino.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C—, ClCH₂—, CF₃CH₂— and CF₃CCl₂—. The terms “halocycloalkyl”, “haloalkoxy”, “haloalkylthio”, “haloalkenyl”, “haloalkynyl”, and the like, are defined analogously to the term “haloalkyl”. Examples of “haloalkoxy” include CF₃O—, CCl₃CH₂O—, HCF₂CH₂CH₂O— and CF₃CH₂O—. Examples of “haloalkylthio” include CCl₃S—, CF₃S—, CCl₃CH₂S— and C₁CH₂CH₂CH₂S—.

“Cyanoalkyl” denotes an alkyl group substituted with one cyano group. Examples of “cyanoalkyl” include NCCH₂, NCCH₂CH₂ and CH₃CH(CN)CH₂. “Hydroxyalkyl” denotes an alkyl group substituted with one hydroxy group. Examples of “hydroxyalkyl” include HOCH₂CH₂, CH₃CH₂(OH)CH and HOCH₂CH₂CH₂CH₂.

The total number of carbon atoms in a substituent group is indicated by the “Cᵢ-Cⱼ” prefix where i and j are numbers from 1 to 10. For example, C₁-C₄ alkylsulfonyl designates methylsulfonyl through butylsulfonyl; C₂ alkoxyalkyl designates CH₃OCH₂—; C₃ alkoxyalkyl designates, for example, CH₃CH(OCH₃)—, CH₃OCH₂CH₂— or CH₃CH₂OCH₂—; and C₄ alkoxyalkyl designates the various isomers of an alkyl group substituted with an alkoxy group containing a total of four carbon atoms, examples including CH₃CH₂CH₂OCH₂— and CH₃CH₂OCH₂CH₂—.

The term “unsubstituted” in connection with a group such as a ring means the group does not have any substituents other than its one or more attachments to the remainder of Formula 1. The term “optionally substituted” means that the number of substituents can be zero. Unless otherwise indicated, optionally substituted groups may be substituted with as many optional substituents as can be accommodated by replacing a hydrogen atom with a non-hydrogen substituent on any available carbon or nitrogen atom. Commonly, the number of optional substituents (when present) range from 1 to 3. As used herein, the term “optionally substituted” is used interchangeably with the phrase “substituted or unsubstituted” or with the term “(un)substituted.”

The number of optional substituents may be restricted by an expressed limitation. For example, the phrase “optionally substituted with up to 3 substituents independently selected from R¹³” means that 0, 1, 2 or 3 substituents can be present (if the number of potential connection points allows). When a range specified for the number of substituents (e.g., x being an integer from 0 to 3 in Exhibit A) exceeds the number of positions available for substituents on a ring (e.g., 1 position available for (R⁶)ₓ on Q-11 in Exhibit A), the actual higher end of the range is recognized to be the number of available positions.

When a compound is substituted with a substituent bearing a subscript that indicates the number of said substituents can vary (e.g., (R⁶)ₓ in Exhibit A wherein x is 1 to 3), then said substituents are independently selected from the group of defined substituents, unless otherwise indicated. Further, when the subscript indicates a range, e.g. (R)ᵢ₋ⱼ, then the number of substituents may be selected from the integers between i and j inclusive. When a group contains a substituent which can be hydrogen, for example R³, then when this substituent is taken as hydrogen, it is recognized that this is equivalent to said group being unsubstituted. When a variable group is shown to be optionally attached to a position, for example (R⁶)ₓ in Exhibit A wherein x may be 0, then hydrogen may be at the position even if not recited in the definition of the variable group. When one or more positions on a group are said to be “not substituted” or “unsubstituted”, then hydrogen atoms are attached to take up any free valency.

Naming of substituents in the present disclosure uses recognized terminology providing conciseness in precisely conveying to those skilled in the art the chemical structure. For sake of conciseness, locant descriptors may be omitted. In some instances herein the point or points of attachment of substituents (e.g., R⁶ and R⁹) are indicated by locant numbers which may be different from the Chemical Abstracts naming system if the difference does not affect the meaning.

Unless otherwise indicated, a “ring” or “ring system” as a component of Formula 1 (e.g., substituent Q) is carbocyclic or heterocyclic. The term “ring system” denotes two or more fused rings. The terms “bicyclic ring system” and “fused bicyclic ring system” denote a ring system consisting of two fused rings, which can be “ortho-fused”, “bridged bicyclic” or “spirocyclic”. An “ortho-fused bicyclic ring system” denotes a ring system wherein the two constituent rings have two adjacent atoms in common. A “bridged bicyclic ring system” is formed by bonding a segment of one or more atoms to nonadjacent ring members of a ring. A “spirocyclic ring system” is formed by bonding a segment of two or more atoms to the same ring member of a ring. The term “ring member” refers to an atom or other moiety (e.g., C(═O), C(═S), S(O) or S(O)₂) forming the backbone of a ring or ring system.

The terms “carbocyclic ring”, “carbocycle” or “carbocyclic ring system” denote a ring or ring system wherein the atoms forming the ring backbone are selected only from carbon. The terms “heterocyclic ring”, “heterocycle” or “heterocyclic ring system” denote a ring or ring system in which at least one atom forming the ring backbone is not carbon, e.g., nitrogen, oxygen or sulfur. Typically a heterocyclic ring contains no more than 4 nitrogen atoms, no more than 2 oxygens and no more than 2 sulfurs. Unless otherwise indicated, a carbocyclic ring or heterocyclic ring can be a saturated or unsaturated ring. “Saturated” refers to a ring having a backbone consisting of atoms linked to one another by single bonds; unless otherwise specified, the remaining atom valences are occupied by hydrogen atoms. Unless otherwise stated, an “unsaturated ring” may be partially unsaturated or fully unsaturated. The expression “fully unsaturated ring” means a ring of atoms in which the bonds between atoms in the ring are single or double bonds according to valence bond theory and furthermore the bonds between atoms in the ring include as many double bonds as possible without double bonds being cumulative (i.e. no C═C═C or C═C═N). The term “partially unsaturated ring” denotes a ring comprising at least one ring member bonded to an adjacent ring member through a double bond and which conceptually potentially accommodates a number of non-cumulated double bonds between adjacent ring members (i.e. in its fully unsaturated counterpart form) greater than the number of double bonds present (i.e. in its partially unsaturated form).

Unless otherwise indicated, heterocyclic rings and ring systems can be attached through any available carbon or nitrogen by replacement of a hydrogen on said carbon or nitrogen.

“Aromatic” indicates that each of the ring atoms is essentially in the same plane and has a p-orbital perpendicular to the ring plane, and in which (4n+2) π electrons, where n is a positive integer, are associated with the ring to comply with Hückel's rule. The term “aromatic ring system” denotes a carbocyclic or heterocyclic ring system in which at least one ring of the ring system is aromatic. When a fully unsaturated carbocyclic ring satisfies Hückel's rule, then said ring is also called an “aromatic ring” or “aromatic carbocyclic ring”.

The term “aromatic carbocyclic ring system” denotes a carbocyclic ring system in which at least one ring of the ring system is aromatic. When a fully unsaturated heterocyclic ring satisfies Hückel's rule, then said ring is also called a “heteroaromatic ring”, “aromatic heterocyclic ring” or “heterocyclic aromatic ring”. The term “aromatic heterocyclic ring system” denotes a heterocyclic ring system in which at least one ring of the ring system is aromatic. The term “nonaromatic ring system” denotes a carbocyclic or heterocyclic ring system that may be fully saturated, as well as partially or fully unsaturated, provided that none of the rings in the ring system are aromatic. The term “nonaromatic carbocyclic ring system” denotes a carbocyclic ring in which no ring in the ring system is aromatic. The term “nonaromatic heterocyclic ring system” denotes a heterocyclic ring system in which no ring in the ring system is aromatic.

The term “optionally substituted” in connection with the heterocyclic rings refers to groups which are unsubstituted or have at least one non-hydrogen substituent that does not extinguish the biological activity possessed by the unsubstituted analog. As used herein, the following definitions shall apply unless otherwise indicated. The term “optionally substituted” is used interchangeably with the phrase “substituted or unsubstituted” or with the term “(un)substituted.” Unless otherwise indicated, an optionally substituted group may have a substituent at each substitutable position of the group, and each substitution is independent of the other.

A wide variety of synthetic methods are known in the art to enable preparation of aromatic and nonaromatic heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Compounds of this invention can exist as one or more stereoisomers. Stereoisomers are isomers of identical constitution but differing in the arrangement of their atoms in space and include enantiomers, diastereomers, cis- and trans-isomers (also known as geometric isomers) and atropisomers. Atropisomers result from restricted rotation about single bonds where the rotational barrier is high enough to permit isolation of the isomeric species. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. For a comprehensive discussion of all aspects of stereoisomerism, see Ernest L. Eliel and Samuel H. Wilen, Stereochemistry of Organic Compounds, John Wiley & Sons, 1994.

Compounds of this invention may be present as a mixture of stereoisomers, individual stereoisomers, or as an optically active form. Unless the structural formula or the language of this application specifically designate a particular cis- or trans-isomer, or a configuration of a chiral center, the scope of the present invention is intended to cover all such isomers per se, as well as mixtures of cis- and trans-isomers, mixtures of diastereomers and racemic mixtures of enantiomers (optical isomers) as well. Molecular depictions drawn herein follow standard conventions for depicting stereochemistry. To indicate stereoconfiguration, bonds rising from the plane of the drawing and towards the viewer are denoted by solid wedges wherein the broad end of the wedge is attached to the atom rising from the plane of the drawing towards the viewer. Bonds going below the plane of the drawing and away from the viewer are denoted by dashed wedges wherein the narrow end of the wedge is attached to the atom further away from the viewer. Constant width lines indicate bonds with a direction opposite or neutral relative to bonds shown with solid or dashed wedges; constant width lines also depict bonds in molecules or parts of molecules in which no particular stereoconfiguration is intended to be specified.

Compounds of Formula 1 can comprise a chiral center at the carbon atom to which the substituents R^{1a} and R^{1b} are attached. For example, when R^{1a} is other than H and R^{1b} is H, the two enantiomers of Formula 1 are depicted below as Formula 1′ and Formula 1″ with the chiral center identified with an asterisk (*).

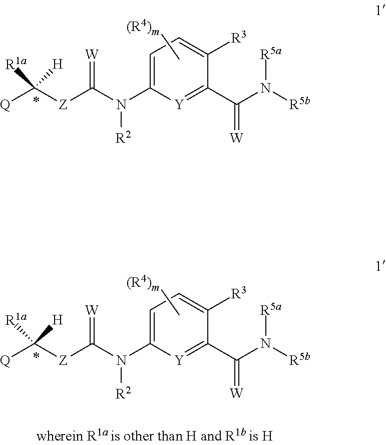

This invention comprises racemic mixtures, for example, equal amounts of the enantiomers of Formulae 1′ and 1″. In addition, this invention includes compounds that are enriched compared to the racemic mixture in an enantiomer of Formula 1. Also included are the essentially pure enantiomers of compounds of Formula 1, for example, Formula 1′ and Formula 1″.

When enantiomerically enriched, one enantiomer is present in greater amounts than the other, and the extent of enrichment can be defined by an expression of enantiomeric excess (“ee”), which is defined as (2x−1)·100%, where x is the mole fraction of the dominant enantiomer in the mixture (e.g., an ee of 20% corresponds to a 60:40 ratio of enantiomers).

Preferably the compositions of this invention have at least a 50% enantiomeric excess; more preferably at least a 75% enantiomeric excess; still more preferably at least a 90% enantiomeric excess; and the most preferably at least a 94% enantiomeric excess of the more active isomer. Of particular note are enantiomerically pure embodiments of the more active isomer.

Compounds of Formula 1 can comprise additional chiral centers. For example, substituents and other molecular constituents such as R^{5a} and R^{5b} may themselves contain chiral centers. This invention comprises racemic mixtures as well as enriched and essentially pure stereoconfigurations at these additional chiral centers.

One skilled in the art will appreciate that not all nitrogen-containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well-known by one skilled in the art including the oxidation of heterocycles and tertiary amines with peroxy acids such as peracetic and 3-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus a wide variety of salts of the compounds of Formula 1 are useful for control of invertebrate pests. The salts of the compounds of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound of Formula 1 contains an acidic moiety such as a carboxylic acid or phenol, salts also include those formed with organic or inorganic bases such as pyridine, triethylamine or ammonia, or amides, hydrides, hydroxides or carbonates of sodium, potassium, lithium, calcium, magnesium or barium. Accordingly, the present invention comprises compounds selected from Formula 1, N-oxides and suitable salts thereof.

Compounds selected from Formula 1, stereoisomers, tautomers, N-oxides, and salts thereof, typically exist in more than one form, and Formula 1 thus includes all crystalline and non-crystalline forms of the compounds that Formula 1 represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due to the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula 1 can exhibit beneficial effects (e.g., suitability for preparation of useful formulations, improved biological performance) relative to another polymorph or a mixture of polymorphs of the same compound represented by Formula 1. Preparation and isolation of a particular polymorph of a compound represented by Formula 1 can be achieved by methods known to those skilled in the art including, for example, crystallization using selected solvents and temperatures. For a comprehensive discussion of polymorphism see R. Hilfiker, Ed., Polymorphism in the Pharmaceutical Industry, Wiley-VCH, Weinheim, 2006.

Embodiments of the present invention as described in the Summary of the Invention include those described below. In the following Embodiments, Formula 1 includes stereoisomers, N-oxides, and salts thereof, and reference to “a compound of Formula 1” includes the definitions of substituents specified in the Summary of the Invention unless further defined in the Embodiments.
Embodiment 1. A compound of Formula 1 wherein Q is a phenyl ring or a naphthalenyl ring system, each ring or ring system optionally substituted with up to 3 substituents independently selected from R⁶; or a 5- to 6-membered heterocyclic ring or an 8- to 11-membered heteroaromatic bicyclic ring system, each ring or ring system containing ring members selected from carbon atoms and 1 to 4 heteroatoms independently selected from up to 2 O, up to 2 S and up to 4 N atoms, wherein up to 2 ring members are independently selected from C(═O), C(═S), S(═O) and S(═O)₂, each ring or ring system optionally substituted with up to 3 substituents independently selected from R⁶.
Embodiment 2. A compound of Formula 1 or Embodiment 1 wherein Q is selected from Q-1 through Q-113 as shown in Exhibit A.

### Exhibit A

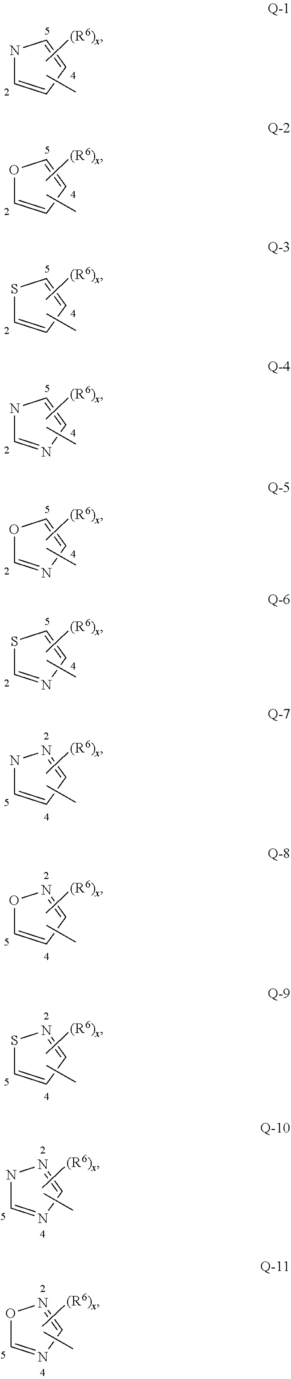 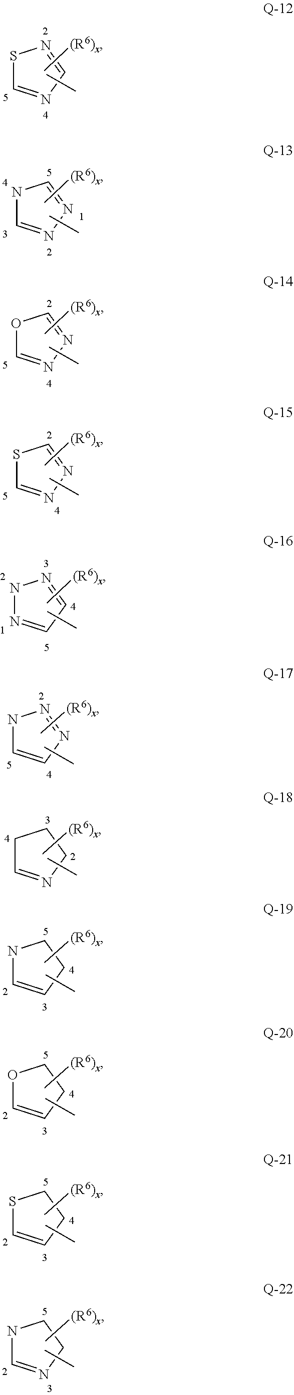 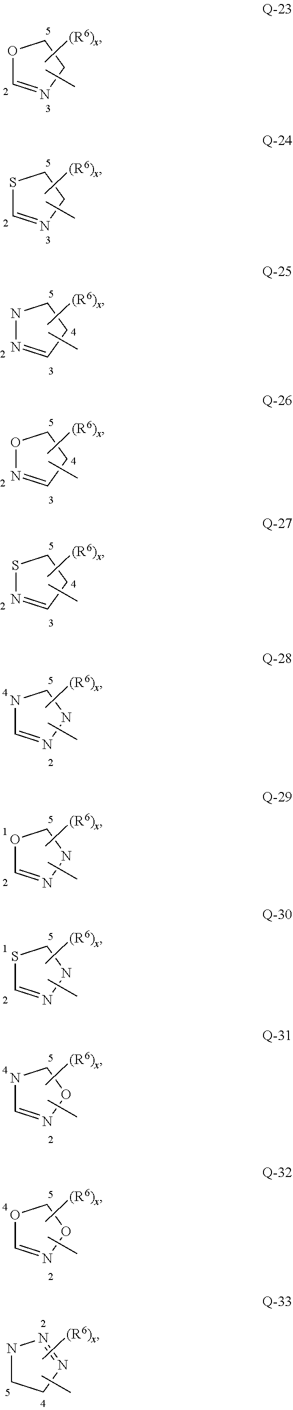 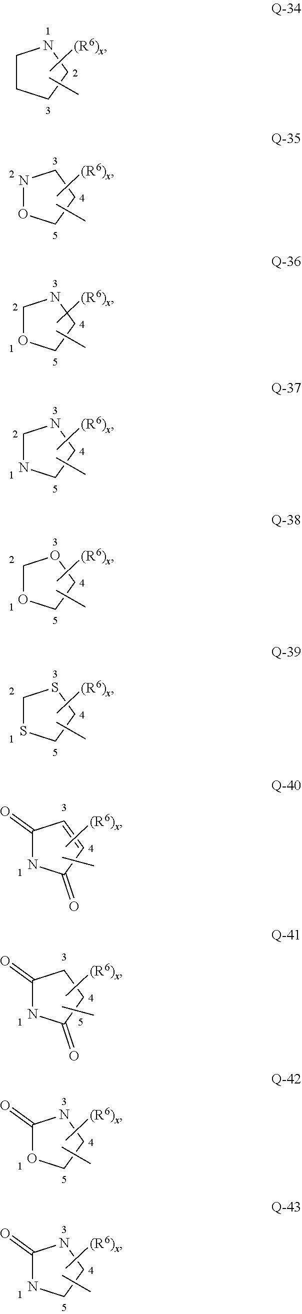 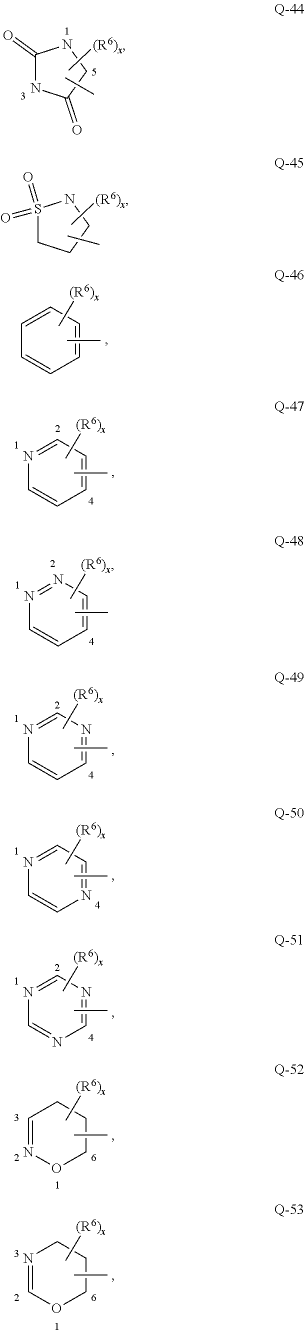 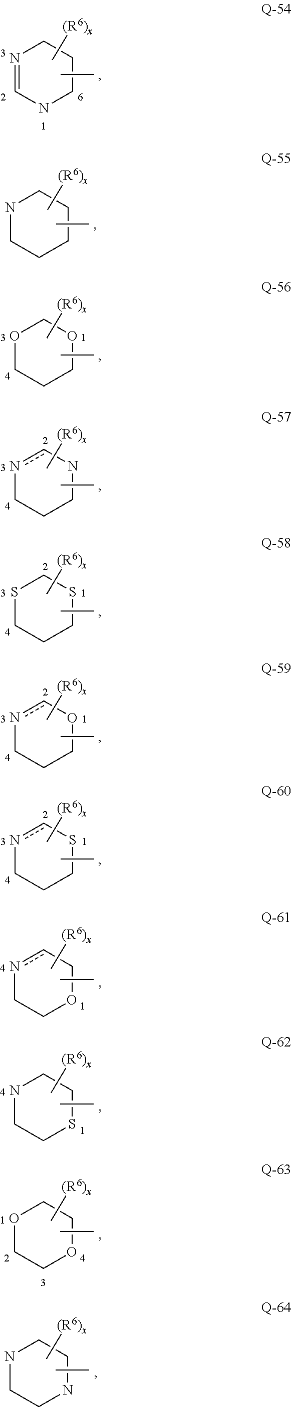 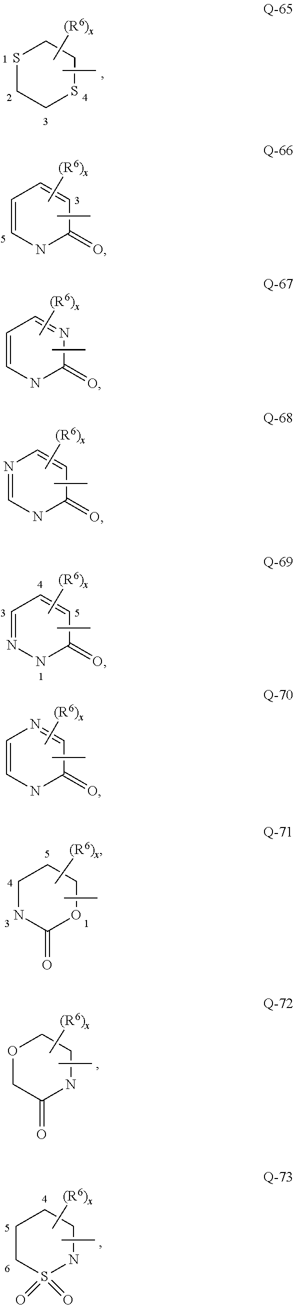 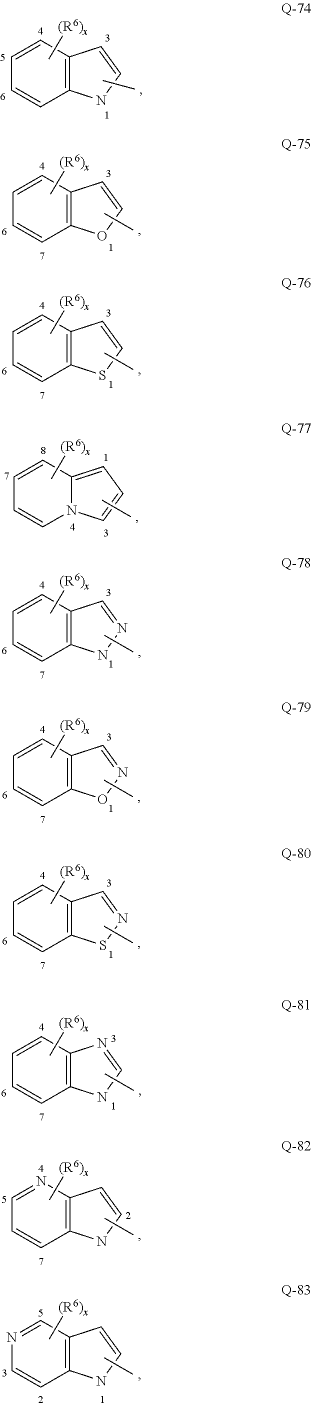 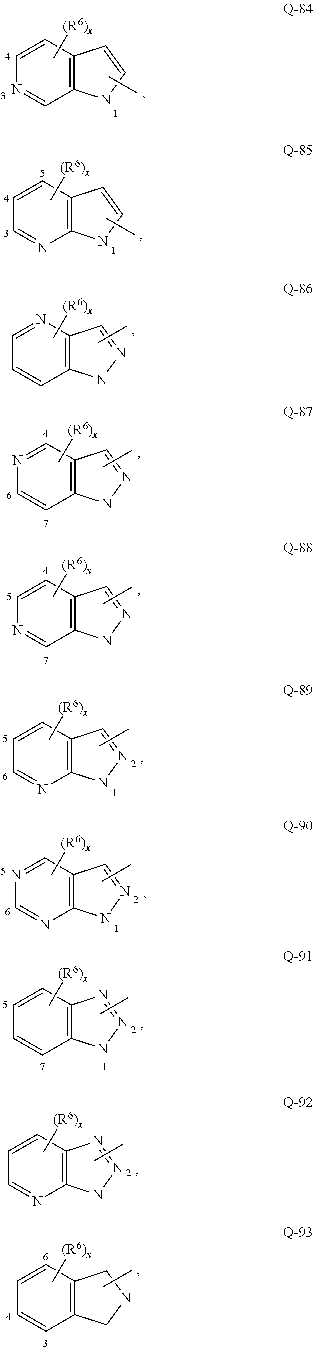 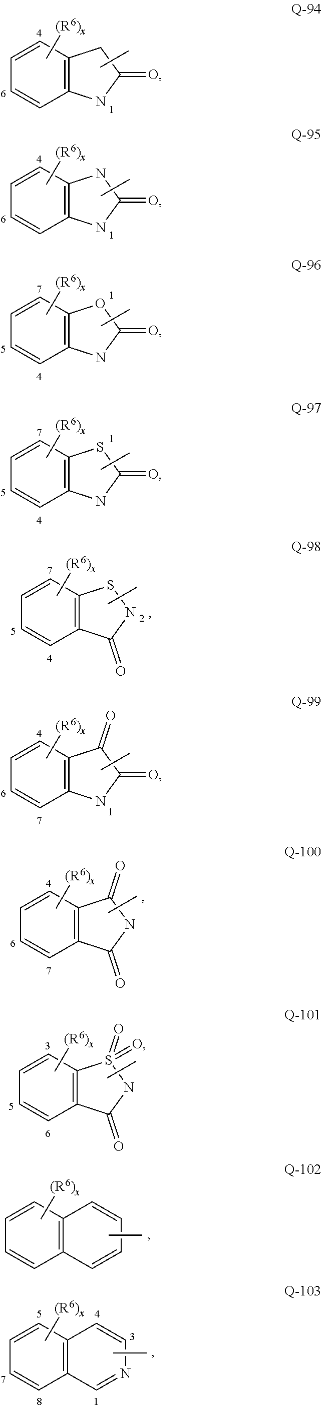 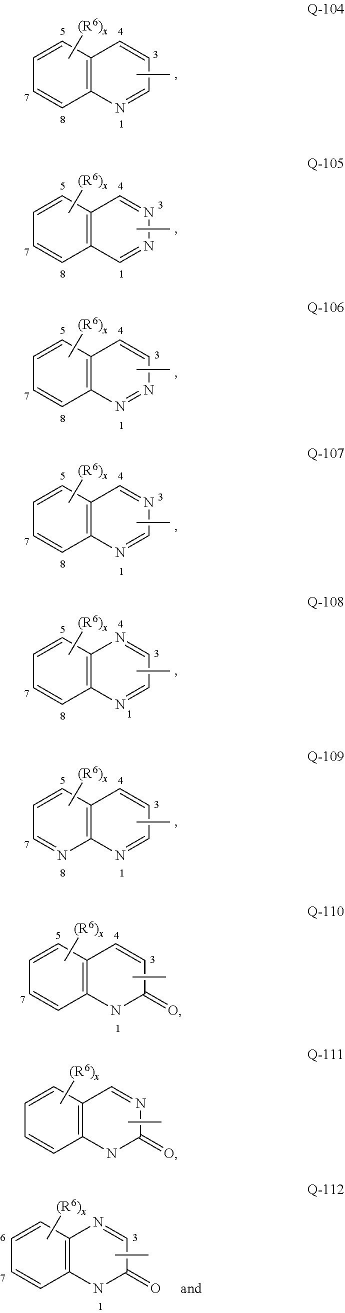 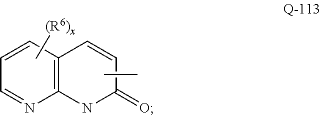 [0109] wherein the floating bond is connected to Formula 1 through any available carbon or nitrogen atom of the depicted ring or ring system; and x is 0, 1, 2 or 3.
Embodiment 3. A compound of Embodiment 2 wherein x is 1, 2 or 3.
Embodiment 4. A compound of Embodiment 2 wherein x is 1 or 2.
Embodiment 5. A compound of Embodiment 2 wherein x is 1.
Embodiment 6. A compound of Embodiment 2 wherein x is 2.
Embodiment 7. A compound of Embodiment 2 wherein x is 3.
Embodiment 8. A compound of anyone of Embodiments 2 through 7 wherein Q is Q-1 through Q-9, Q-46 through Q-50 or Q-66 through Q-70.
Embodiment 9. A compound of Embodiment 8 wherein Q is Q is Q-1 through Q-9.
Embodiment 9a. A compound of Embodiment 8 wherein Q is Q-46 through Q-50.
Embodiment 10. A compound of Embodiment 9a wherein Q is Q-46, Q-47, Q-49 or Q-50.
Embodiment 11. A compound of Embodiment 10 wherein Q is Q-46 or Q-47.
Embodiment 12. A compound of Embodiment 11 wherein Q is Q-46.
Embodiment 13. A compound of Formula 1 or anyone of Embodiments 1 through 12 wherein Q is Q-46, x is 3 and R⁶ is attached at the 3-, 4- and 5-positions of Q-46 (i.e. at the ortho position and both meta positions), relative to the connection of the Q-46 ring to the remainder of Formula 1.
Embodiment 14. A compound of Formula 1 or anyone of Embodiments 1 through 12 wherein Q is Q-46, x is 2 and R⁶ is attached at the 3- and 5-positions of Q-46 (i.e. both meta positions), relative to the connection of the Q-46 ring to the remainder of Formula 1.
Embodiment 15. A compound of Formula 1 or anyone of Embodiments 1 through 12 wherein Q is Q-46, x is 2 and R⁶ is attached at the 3- and 4-positions of Q-46 (i.e. at the ortho position and a meta position), relative to the connection of the Q-46 ring to the remainder of Formula 1.
Embodiment 16. A compound of Formula 1 or anyone of Embodiments 1 through 12 wherein Q is Q-46, x is 1 and R⁶ is attached at the 3-position of Q-46 (i.e. at a meta position), relative to the connection of the Q-46 ring to the remainder of Formula 1.
Embodiment 17. A compound of Formula 1 or anyone of Embodiments 1 through 12 wherein Q is Q-46, x is 1 and R⁶ is attached at the 4-position of Q-46 (i.e. at the para position), relative to the connection of the Q-46 ring to the remainder of Formula 1.
Embodiment 18. A compound of Formula 1 or anyone of Embodiments 1 through 17 wherein Q is Q-46 substituted at the 3-, 4- and 5-positions (i.e. at the ortho position and both meta positions) with substituents independently selected from R⁶; or Q is Q-46 substituted at the 3- and 4-positions (i.e. at the ortho position and a meta position) with substituents independently selected from R⁶; or Q is Q-46 substituted at the 3- and 5-positions (i.e. both meta positions) with substituents independently selected from R⁶; or Q is Q-46 substituted at the 3-position (i.e. a meta position) with a substituent selected from R⁶.
Embodiment 19. A compound of Embodiment 18 wherein Q is Q-46 substituted at the 3- and 4-positions (i.e. at the ortho position and a meta position) with substituents independently selected from R⁶; or Q is Q-46 substituted at the 3- and 5-positions (i.e. both meta positions) with substituents independently selected from R⁶; or Q is Q-46 substituted at the 3-position (i.e. a meta position) with a substituent selected from R⁶.
Embodiment 20. A compound of Embodiment 19 wherein Q is Q-46 substituted at the 3- and 5-positions (i.e. both meta positions) with substituents independently selected from R⁶; or Q is Q-46 substituted at the 3-position (i.e. a meta position) with a substituent selected from R⁶.
Embodiment 20a. A compound of Embodiment 19 wherein Q is phenyl substituted at the 3- and 4-positions (i.e. at the ortho position and a meta position) with substituents independently selected from R⁶.
Embodiment 20b. A compound of Embodiment 20 wherein Q is Q-46 substituted at the 3- and 5-positions (i.e. both meta positions) with substituents independently selected from R⁶.
Embodiment 20c. A compound of Embodiments 20 wherein Q is Q-46 substituted at the 3-position (i.e. a meta position) with a substituent selected from R⁶.
Embodiment 21a. A compound of Formula 1 or any one of Embodiments 1 through 20c wherein Y is CR⁴ and R^{1a} is CF₃, CHF₂, CCl₃, CF₂Cl, CFCl₂, or CHFCl.
Embodiment 21b. A compound of Formula 1 or any one of Embodiments 1 through 20c wherein Y is N and R^{1a} is CF₃, CHF₂, CCl₃, CF₂Cl, CFCl₂, or CHFCl.
Embodiment 21c. A compound of Formula 1 or any one of Embodiments 1 through 20c wherein Y is CR⁴ and R^{1a} is CF₃, CHF₂, CCl₃, CF₂Cl, or CFCl₂.
Embodiment 21d. A compound of Formula 1 or any one of Embodiments 1 through 20c wherein Y is CR⁴ and R^{1a} is CF₃, CHF₂, CCl₃, CF₂Cl, or CFCl₂.
Embodiment 22. A compound of any one of Embodiments 21a through 21d wherein R^{1a} is CF₃, CHF₂, CCl₃ or CF₂C₁.
Embodiment 23a. A compound of Embodiment 22 wherein R^{1a} is CF₃.
Embodiment 23b. A compound of Embodiment 22 wherein R^{1a} is CHF₂.
Embodiment 24. A compound of Formula 1 or any one of Embodiments 1 through 23b wherein R^{1b} is H, halogen, hydroxy, methyl, halomethyl, C₁-C₂ alkoxy or C₁-C₂ haloalkoxy.
Embodiment 25. A compound of Embodiment 24 wherein R^{1b} is H, halogen, hydroxy, methyl, halomethyl, methoxy or halomethoxy.
Embodiment 25a. A compound of Embodiment 25 wherein R^{1b} is H, Br, Cl, F, hydroxy, methyl, halomethyl, methoxy or halomethoxy.
Embodiment 26. A compound of Embodiment 25a wherein R^{1b} is H, Br, Cl, F, hydroxy, methyl or halomethyl.
Embodiment 27. A compound of Embodiment 26 wherein R^{1b} is H, Br, Cl, F, hydroxy or methyl.
Embodiment 28. A compound of Embodiment 27 wherein R^{1b} is H, hydroxy or methyl.
Embodiment 29. A compound of Embodiment 28 wherein R^{1b} is H.
Embodiment 29a. A compound of Formula 1 or any one of Embodiments 1 through 29 wherein R^{1a} is CF₃ and R^{1b} is H.
Embodiment 30. A compound of Formula 1 or any one of Embodiments 1 through 29a wherein Z is CR^{7a}R^{7b}, NR^{7c} or O.
Embodiment 31a. A compound of Embodiment 30 wherein Z is CR^{7a}R^{7b} or NR^{7c}.
Embodiment 31b. A compound of Embodiment 31a wherein Z is NR^{7c}.
Embodiment 31c. A compound of Embodiment 31b wherein Z is NH.
Embodiment 31d. A compound of Embodiment 30 wherein Z is O.
Embodiment 32. A compound of Embodiment 30 wherein Z is CR^{7a}R^{7b}.
Embodiment 33. A compound of Formula 1 or any one of Embodiments 1 through 32 wherein each W is O.
Embodiment 34. A compound of Formula 1 or any one of Embodiments 1 through 33 wherein R² is H; or C₁-C₆ alkyl, C₂-C₆ alkenyl, C₂-C₆ alkylcarbonyl or C₂-C₆ alkoxycarbonyl, each optionally substituted with up to 3 substituents independently selected from halogen, cyano, C₁-C₃ alkyl, C₁-C₃ haloalkyl or C₁-C₃ alkoxy.
Embodiment 35. A compound of Embodiment 34 wherein R² is H; or C₁-C₃ alkyl, C₂-C₃ alkenyl, C₂-C₃ alkylcarbonyl or C₂-C₃ alkoxycarbonyl, each optionally substituted with up to 3 substituents independently selected from Br, Cl, F, C₁-C₃ alkyl, C₁-C₃ haloalkyl or C₁-C₃ alkoxy.
Embodiment 36. A compound of Embodiment 35 wherein R² is H; or C₁-C₃ alkyl, C₂-C₃ alkenyl, C₂-C₃ alkylcarbonyl or C₂-C₃ alkoxycarbonyl, each optionally substituted with up to 3 substituents independently selected from Br, Cl, F or methyl.
Embodiment 37. A compound of Embodiment 36 wherein R² is H, C₁-C₃ alkyl, C₂-C₃ alkenyl, C₂-C₃ alkylcarbonyl or C₂-C₃ alkoxycarbonyl.
Embodiment 38. A compound of Embodiment 37 wherein R² is H or C₁-C₃ alkyl.
Embodiment 39. A compound of Embodiment 38 wherein R² is H or methyl.
Embodiment 40. A compound of Embodiment 39 wherein R² is H.
Embodiment 41. A compound of Formula 1 or any one of Embodiments 1 through 40 wherein R³ is H, halogen, cyano, C₁-C₃ alkyl, C₁-C₃ haloalkyl, OR⁸ or S(═O)ₜR⁸.
Embodiment 42. A compound of Formula 1 or Embodiment 41 wherein each t is independently 0 or 2.
Embodiment 43. A compound of Embodiment 42 wherein each t is 0.
Embodiment 44. A compound of Embodiment 42 wherein each t is 2.
Embodiment 45. A compound of Embodiment 41 wherein R³ is H, halogen, C₁-C₃ alkyl, C₁-C₃ haloalkyl or OR⁸.
Embodiment 46. A compound of Embodiment 45 wherein R³ is H, halogen or C₁-C₃ alkyl.
Embodiment 47. A compound of Embodiment 46 wherein R³ is H or halogen.
Embodiment 48. A compound of Embodiment 47 wherein R³ is H, Br, Cl or F.
Embodiment 49. A compound of Embodiment 48 wherein R³ is H, Cl or F.
Embodiment 50. A compound of Embodiment 47 wherein R³ is Br, Cl or F.
Embodiment 51. A compound of Embodiment 50 wherein R³ is Cl or F.
Embodiment 52. A compound of Embodiment 51 wherein R³ is C₁.
Embodiment 53. A compound of Formula 1 or any one of Embodiments 1 through 52 wherein each R⁴ is independently halogen, cyano or C₁-C₂ alkyl.
Embodiment 54. A compound of Embodiment 53 wherein each R⁴ is independently Br, Cl, F, cyano or methyl.
Embodiment 55. A compound of Embodiment 54 wherein each R⁴ is independently Br Cl or F.
Embodiment 56. A compound of Embodiment 55 wherein each R⁴ is independently Cl or F.
Embodiment 57. A compound of Embodiment 56 wherein each R⁴ is F Embodiment 58. A compound of Formula 1 or any one of Embodiments 1 through 57 wherein m is 0, 1 or 2.
Embodiment 59. A compound of Embodiment 58 wherein m is 0 or 1.
Embodiment 60. A compound of Embodiment 59 wherein m is 0.
Embodiment 61. A compound of Embodiment 59 wherein m is 1.
Embodiment 62. A compound of Formula 1 or any one of Embodiments 1 through 61 wherein when R^{5a} is separate (i.e. not taken together with R^{5b} to form a ring), then R^{5a} is H, cyano, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₂-C₆ alkoxyalkyl, C₂-C₆ haloalkoxyalkyl, C₁-C₆ alkylsulfonyl, C₂-C₆ alkylcarbonyl, C₂-C₆ haloalkylcarbonyl, C₂-C₆ alkoxycarbonyl, C₂-C₆ alkylaminocarbonyl or C₃-C₆ dialkylaminocarbonyl.
Embodiment 63. A compound of Embodiment 62 wherein R^{5a} is H, C₁-C₆ alkyl, C₁-C₆ haloalkyl, C₂-C₆ alkenyl, C₂-C₆ haloalkenyl, C₂-C₆ alkoxyalkyl, C₂-C₆ haloalkoxyalkyl, C₂-C₆ alkylcarbonyl, C₂-C₆ haloalkylcarbonyl or C₂-C₆ alkoxycarbonyl.
Embodiment 64. A compound of Embodiment 63 wherein R^{5a} is H, C₁-C₆ alkyl, C₁-C₆ haloalkyl, C₂-C₆ alkenyl, C₂-C₆ alkoxyalkyl, C₂-C₆ alkylcarbonyl or C₂-C₆ alkoxycarbonyl.
Embodiment 65. A compound of Embodiment 64 wherein R^{5a} is H, C₁-C₆ alkyl, C₁-C₆ haloalkyl, C₂-C₆ alkoxyalkyl, C₂-C₆ alkylcarbonyl or C₂-C₆ alkoxycarbonyl.
Embodiment 66. A compound of Embodiment 65 wherein R^{5a} is H, C₁-C₆ alkyl or C₁-C₆ haloalkyl.
Embodiment 67. A compound of Embodiment 66 wherein R^{5a} is H, C₁-C₃ alkyl or C₁-C₃ haloalkyl.
Embodiment 68. A compound of Embodiment 67 wherein R^{5a} is H, methyl or halomethyl.
Embodiment 69. A compound of Embodiment 68 wherein R^{5a} is H or methyl.
Embodiment 70. A compound of Embodiment 69 wherein R^{5a} is H.
Embodiment 71. A compound of Formula 1 or any one of Embodiments 1 through 70 wherein when R^{5b} is separate (i.e. not taken together with R^{5a} to form a ring), then R^{5b} is H, C₁-C₆ alkyl, C₁-C₆ haloalkyl, C₂-C₆ alkenyl, C₂-C₆ haloalkenyl, C₂-C₆ alkynyl, C₂-C₆ cyanoalkyl, C₃-C₆ cycloalkyl, C₃-C₆ halocycloalkyl, C₄-C₁₀ alkylcycloalkyl, C₄-C₁₀ cycloalkylalkyl, C₄-C₁₀ halocycloalkylalkyl, C₂-C₆ alkoxyalkyl, C₂-C₆ haloalkoxyalkyl, C₂-C₆ alkylaminoalkyl, C₂-C₆ haloalkylaminoalkyl or C₃-C₈ dialkylaminoalkyl; or a phenyl ring optionally substituted with up to 3 substituents independently selected from R⁹; or a 5- to 6-membered heterocyclic ring or an 8- to 11-membered heteroaromatic bicyclic ring system, each ring or ring system containing ring members selected from carbon atoms and 1 to 4 heteroatoms independently selected from up to 2 O, up to 2 S and up to 4 N atoms, wherein up to 2 ring members are independently selected from C(═O), C(═S), S(═O) and S(═O)₂, each ring optionally substituted with up to 3 substituents independently selected from R⁹; or -A(CR^{10a}R^{10b})ₙB or NR^{21a}R^{21b}.
Embodiment 72. A compound of Embodiment 71 wherein R^{5b} is H, C₁-C₆ alkyl, C₁-C₆ haloalkyl, C₂-C₆ cyanoalkyl, C₃-C₆ cycloalkyl or C₄-C₁₀ cycloalkylalkyl; or a phenyl ring optionally substituted with up to 3 substituents independently selected from R⁹; or a 5- to 6-membered heterocyclic ring, each ring containing ring members selected from carbon atoms and 1 to 4 heteroatoms independently selected from up to 2 O, up to 2 S and up to 4 N atoms, wherein up to 2 ring members are independently selected from C(═O), C(═S), S(═O) and S(═O)₂, each ring optionally substituted with up to 3 substituents independently selected from R⁹; or -A(CR^{10a}R^{10b})ₙB or NR^{21a}R^{21b}.
Embodiment 73. A compound of Embodiment 72 wherein R^{5b} is H, C₁-C₆ haloalkyl or C₃-C₆ cycloalkyl; or a phenyl ring optionally substituted with up to 3 substituents independently selected from R⁹; or a 6-membered heterocyclic ring, containing ring members selected from carbon atoms and 1 to 4 heteroatoms independently selected from up to 2 O, up to 2 S and up to 4 N atoms, wherein up to 2 ring members are independently selected from C(═O), C(═S), S(═O) and S(═O)₂, each ring optionally substituted with up to 3 substituents independently selected from R⁹.
Embodiment 73a. A compound of Embodiment 73 wherein R^{5b} is H, C₁-C₆ haloalkyl or C₃-C₆ cycloalkyl; or a phenyl ring optionally substituted with up to 3 substituents independently selected from R⁹; or a 6-membered heterocyclic ring, containing ring members selected from carbon atoms and 1 to 2 heteroatoms independently selected from up to 2 O, up to 2 S and up to 2 N atoms, each ring optionally substituted with up to 3 substituents independently selected from R⁹.
Embodiment 74. A compound of Embodiment 73a wherein R^{5b} is C₁-C₆ haloalkyl or C₃-C₆ cycloalkyl; or a phenyl ring optionally substituted with up to 3 substituents independently selected from R⁹; or a pyridinyl, pyrimidinyl, pyrazinyl or pyridazinyl ring, each ring optionally substituted with up to 3 substituents independently selected from R⁹.
Embodiment 75. A compound of Embodiment 74 wherein R^{5b} is C₁-C₆ haloalkyl or C₃-C₆ cycloalkyl; or a phenyl or pyridinyl ring, each ring optionally substituted with up to 3 substituents independently selected from R⁹.
Embodiment 76. A compound of Embodiment 75 wherein R^{5b} is C₁-C₃ haloalkyl or cyclopropyl; or a phenyl or pyridinyl ring, each ring optionally substituted with up to 3 substituents independently selected from R⁹.
Embodiment 76a. A compound of Embodiment 76 wherein R^{5b} is C₁-C₃ fluoroalkyl or cyclopropyl; or a phenyl or pyridinyl ring, each ring optionally substituted with up to 3 substituents independently selected from R⁹.
Embodiment 77. A compound of Embodiment 76a wherein R^{5b} is CH₂CF₃ or cyclopropyl; or a phenyl or pyridinyl ring, each ring optionally substituted with up to 3 substituents independently selected from R⁹.
Embodiment 78. A compound of Embodiment 77 wherein R^{5b} is CH₂CF₃ or cyclopropyl; or a phenyl, 2-pyridinyl or 3-pyridinyl ring, each ring optionally substituted with up to 3 substituents independently selected from R⁹.
Embodiment 79. A compound of Embodiment 78 wherein R^{5b} is cyclopropyl; or a phenyl, 2-pyridinyl or 3-pyridinyl ring, each ring optionally substituted with up to 3 substituents independently selected from R⁹.
Embodiment 79a. A compound of Embodiment 79 wherein R^{5b} is cyclopropyl; or a phenyl, 2-pyridinyl or 3-pyridinyl ring, each ring optionally substituted with up to 2 substituents independently selected from R⁹
Embodiment 79b. A compound of Embodiment 79a wherein R^{5b} is cyclopropyl; or a phenyl or 2-pyridinyl ring, each ring optionally substituted with up to 2 substituents independently selected from R⁹.
Embodiment 80. A compound of Embodiment 79 wherein R^{5b} is a phenyl ring optionally substituted with up to 3 substituents independently selected from R⁹.
Embodiment 81. A compound of Embodiment 80 wherein R^{5b} is a phenyl ring optionally substituted with up to 2 substituents independently selected from R⁹.
Embodiment 82. A compound of Embodiment 81 wherein R^{5b} is a phenyl ring optionally substituted with up to 1 substituent selected from R⁹.
Embodiment 81. A compound of Formula 1 or anyone of Embodiments 1 through 82 wherein when R^{5b} is separate (i.e. not taken together with R^{5a} to form a ring), then R^{5b} is a phenyl ring substituted at the 2-, 4- and 6-positions with substituents independently selected from R⁹; or a phenyl ring substituted at the 2- and 4-positions with substituents independently selected from R⁹; or a phenyl ring substituted at the 4-position with a substituent selected from R⁹.
Embodiment 82. A compound of Embodiment 81 wherein when R^{5b} a phenyl ring substituted at the 2- and 4-positions with substituents independently selected from R⁹; or a phenyl ring substituted at the 4-position with a substituent selected from R⁹.
Embodiment 83. A compound of Formula 1 or any one of Embodiments 1 through 82 wherein when R^{5a} and R^{5b} are taken together to form a ring (i.e. R^{5a} and R^{5b} are not separate), then said ring is a 5- to 6-membered fully saturated heterocyclic ring, each ring containing ring members, in addition to the connecting nitrogen atom, selected from carbon atoms and up to 2 heteroatoms independently selected from up to 2 O, up to 2 S and up to 2 N atoms, each ring optionally substituted with up to 3 substituents independently selected from R¹³.
Embodiment 84. A compound of Embodiment 83 wherein R^{5a} and R^{5b} are taken together to form a ring a 5- to 6-membered fully saturated heterocyclic ring, each ring containing ring members, in addition to the connecting nitrogen atom, selected from carbon atoms and up to 2 heteroatoms independently selected from up to 2 O, up to 2 S and up to 2 N atoms, each ring optionally substituted with up to 2 substituents independently selected from R¹³.
Embodiment 85. A compound of Formula 1 or any one of Embodiments 1 through 84 wherein A is O or direct bond.
Embodiment 86. A compound of Embodiment 85 wherein A is O.
Embodiment 87. A compound of Embodiment 85 wherein A is direct bond.
Embodiment 88. A compound of Formula 1 or any one of Embodiments 1 through 84 wherein A is NR¹¹.
Embodiment 89. A compound of Formula 1 or any one of Embodiments 1 through 88 wherein n is 0, 1 or 2.
Embodiment 90. A compound of Formula 1 or any one of Embodiments 1 through 82 wherein n is 0 or 1.
Embodiment 91. A compound of Embodiment 90 wherein n is 0.
Embodiment 92. A compound of Embodiment 90 wherein n is 1.
Embodiment 93. A compound of Formula 1 or any one of Embodiments 1 through 92 wherein B is a phenyl ring optionally substituted with up to 3 substituents independently selected from R¹²; or a 5- to 6-membered heterocyclic ring containing ring members selected from carbon atoms and 1 to 4 heteroatoms independently selected from up to 2 O, up to 2 S and up to 4 N atoms, the ring optionally substituted with up to 3 substituents independently selected from R¹².
Embodiment 94. A compound of Embodiment 93 wherein B is a phenyl ring optionally substituted with up to 3 substituents independently selected from R¹².
Embodiment 95. A compound of Embodiment 94 wherein B is a phenyl ring optionally substituted with up to 2 substituents independently selected from R¹².
Embodiment 96. A compound of Formula 1 or any one of Embodiments 1 through 95 wherein each R⁶ is independently cyano, halogen, nitro, NR^{14a}R^{14b}, C(═O)NR^{14a}R^{14b}, C(═S)NR^{14a}R^{14b} or —U—V-T; or C₁-C₆ alkyl, C₁-C₆ haloalkyl, C₂-C₆ alkenyl, C₂-C₆ haloalkenyl, C₃-C₆ cycloalkyl, C₃-C₆ halocycloalkyl, C₂-C₆ cyanoalkyl, C₄-C₁₀ alkylcycloalkyl, C₄-C₁₀ cycloalkylalkyl, C₂-C₆ alkoxyalkyl, C₂-C₆ alkylthioalkyl, C₁-C₆ alkoxy, C₁-C₆ haloalkoxy, C₂-C₆ alkenyloxy, C₂-C₆ haloalkenyloxy, C₂-C₆ alkoxyalkoxy, C₁-C₆ alkylthio, C₁-C₆ alkylamino, C₂-C₆ dialkylamino or C₁-C₆ haloalkylamino, each optionally substituted with up to 3 substituents independently selected from R¹⁵.
Embodiment 97. A compound of Embodiment 96 wherein each R⁶ is independently cyano, halogen, NR^{14a}R^{14b}, C(═O)NR^{14a}R^{14b} or —U—V-T; or C₁-C₆ alkyl, C₁-C₆ haloalkyl, C₂-C₆ alkenyl, C₂-C₆ haloalkenyl, C₂-C₆ cyanoalkyl, C₂-C₆ alkoxyalkyl, C₁-C₆ alkoxy, C₁-C₆ haloalkoxy, C₂-C₆ alkenyloxy, C₂-C₆ haloalkenyloxy, C₁-C₆ alkylthio, C₁-C₆ alkylamino or C₂-C₆ dialkylamino, each optionally substituted with up to 1 substituent selected from R¹⁵.
Embodiment 98. A compound of Embodiment 97 wherein each R⁶ is independently cyano, halogen, C₁-C₆ alkyl, C₁-C₆ haloalkyl, C₂-C₆ alkenyl, C₂-C₆ haloalkenyl, C₁-C₆ alkoxy, C₁-C₆ haloalkoxy or C₂-C₆ alkenyloxy.
Embodiment 99. A compound of Embodiment 98 wherein each R⁶ is independently halogen, C₁-C₃ alkyl, C₁-C₃ haloalkyl, C₁-C₃ alkoxy, C₁-C₃ haloalkoxy or C₂-C₄ alkenyloxy.
Embodiment 100. A compound of Embodiment 99 wherein each R⁶ is independently halogen, C₁-C₃ alkyl, C₁-C₃ haloalkyl or C₁-C₃ alkoxy.
Embodiment 101. compound of Embodiment 100 wherein each R⁶ is independently Br, Cl, F or trifluoromethyl.
Embodiment 102. compound of Embodiment 101 wherein each R⁶ is independently Cl, F or trifluoromethyl.
Embodiment 103. A compound of Formula 1 or any one of Embodiments 1 through 102 wherein R^{7a} is H, C₁-C₃ alkyl, C₁-C₃ haloalkyl or cyclopropyl.
Embodiment 104. A compound of Embodiment 103 wherein R^{7a} is H, methyl, trifluoromethyl or cyclopropyl.
Embodiment 105. A compound of Embodiment 104 wherein R^{7a} is H or methyl.
Embodiment 106. A compound of Embodiment 105 wherein R^{7a} is H.
Embodiment 107. A compound of Formula 1 or any one of Embodiments 1 through 106 wherein R^{7b} is H, C₁-C₃ alkyl, C₁-C₃ haloalkyl, C₂-C₄ cyanoalkyl, C₃-C₆ cycloalkyl, C₃-C₆ halocycloalkyl, C₄-C₆ alkylcycloalkyl, C₄-C₆ cycloalkylalkyl, C₄-C₆ halocycloalkylalkyl, C₂-C₃ alkoxyalkyl, C₂-C₃ alkylaminoalkyl; or phenyl optionally substituted with up to 3 substituents independently selected from R¹⁶.
Embodiment 108. A compound of Embodiment 107 wherein R^{7b} is H, C₁-C₃ alkyl, C₁-C₃ haloalkyl, C₃-C₆ cycloalkyl, C₃-C₆ halocycloalkyl, C₂-C₃ alkoxyalkyl; or phenyl optionally substituted with up to 3 substituents independently selected from R¹⁶.
Embodiment 109. A compound of Embodiment 108 wherein R^{7b} is H, C₁-C₃ alkyl, C₁-C₃ haloalkyl; or phenyl optionally substituted with up to 3 substituents independently selected from R¹⁶.
Embodiment 110. A compound of Embodiment 109 wherein R^{7b} is H, C₁-C₃ alkyl or C₁-C₃ haloalkyl.
Embodiment 111. A compound of Embodiment 110 wherein R^{7b} is H, methyl or trifluoromethyl.
Embodiment 112. A compound of Embodiment 111 wherein R^{7b} is H or methyl.
Embodiment 113. A compound of Embodiment 112 wherein R^{7b} is H.
Embodiment 114. A compound of Formula 1 or any one of Embodiments 1 through 113 wherein R^{7c} is H, methyl, trifluoromethyl or C₂-C₄ alkylcarbonyl.
Embodiment 115. A compound of Embodiment 114 wherein R^{7c} is H or methyl.
Embodiment 116. A compound of Embodiment 115 wherein R^{7c} is H.
Embodiment 117. A compound of Formula 1 or any one of Embodiments 1 through 116 wherein each R⁸ is independently H, C₁-C₃ alkyl, C₁-C₃ haloalkyl, C₂-C₄ alkenyl or C₃-C₆ cycloalkyl; or phenyl or benzyl each ring optionally substituted with up to 3 substituents independently selected from R¹⁷.
Embodiment 118. A compound of Embodiment 117 wherein each R⁸ is independently H, C₁-C₃ alkyl or C₁-C₃ haloalkyl; or phenyl optionally substituted with up to 3 substituents independently selected from R¹⁷.
Embodiment 119. A compound of Embodiment 118 wherein each R⁸ is independently H or C₁-C₃ alkyl.
Embodiment 120. A compound of Embodiment 119 wherein each R⁸ is independently C₁-C₂ alkyl.
Embodiment 121. A compound of Embodiment 120 wherein each R⁸ is methyl.
Embodiment 122. A compound of Formula 1 or any one of Embodiments 1 through 121 wherein each R⁹ is independently cyano, halogen, nitro, C₁-C₃ alkyl, C₁-C₃ haloalkyl, cyclopropyl, C₁-C₃ alkoxy, C₁-C₃ haloalkoxy, C₂-C₄ alkoxyalkoxy, C₁-C₃ alkylthio, C₂-C₄ alkylcarbonyl, C₂-C₄ alkoxycarbonyl, C₁-C₃ alkylamino or C₂-C₄ dialkylamino.
Embodiment 123. A compound of Embodiment 122 wherein each R⁹ is independently cyano, halogen, nitro, C₁-C₃ alkyl, C₁-C₃ haloalkyl, C₁-C₃ alkoxy or C₁-C₃ haloalkoxy.
Embodiment 124. A compound of Embodiment 123 wherein each R⁹ is independently halogen, methyl, trifluoromethyl or methoxy.
Embodiment 125. A compound of Embodiment 124 wherein each R⁹ is independently Br, Cl, F or trifluoromethyl.
Embodiment 126. A compound of Embodiment 125 wherein each R⁹ is independently Cl, F or trifluoromethyl.
Embodiment 127. A compound of Embodiment 126 wherein each R⁹ is independently C₁ or F.
Embodiment 128. A compound of Formula 1 or any one of Embodiments 1 through 127 wherein each R^{10a} is independently H, Br, Cl, F, cyano or methyl.
Embodiment 129. A compound of Embodiment 128 wherein each R^{10a} is independently H, Br, Cl, F or methyl.
Embodiment 130. A compound of Embodiment 129 wherein each R^{10a} is H.
Embodiment 131. A compound of Formula 1 or any one of Embodiments 1 through 130 wherein each R^{10b} is independently H or methyl.
Embodiment 132. A compound of Embodiment 131 wherein each R^{10b} is H.
Embodiment 133. A compound of Formula 1 or any one of Embodiments 1 through 132 wherein R¹¹ is H, cyano, methyl or methoxy.
Embodiment 134. A compound of Embodiment 133 wherein R¹¹ is H.
Embodiment 135. A compound of Formula 1 or any one of Embodiments 1 through 134 wherein each R¹² is independently halogen, methyl, halomethyl or methoxy.
Embodiment 136. A compound of Embodiment 120 wherein each R¹² is independently halogen or methyl.
Embodiment 137. A compound of Formula 1 or any one of Embodiments 1 through 136 wherein each R¹³ is independently halogen, cyano, C₁-C₆ alkyl, C₁-C₆ haloalkyl, C₂-C₆ alkenyl, C₂-C₆ haloalkenyl, C₃-C₆ cycloalkyl, C₃-C₆ halocycloalkyl, C₂-C₆ alkoxyalkyl, C₂-C₆ haloalkoxyalkyl, C₁-C₆ alkoxy, C₁-C₆ haloalkoxy, C₃-C₆ cycloalkoxy, C₂-C₆ alkenyloxy, C₂-C₆ haloalkenyloxy, C₁-C₆ alkylthio, C₁-C₆ haloalkylthio, C₂-C₆ alkylcarbonyl, C₂-C₆ haloalkylcarbonyl, C₂-C₆ alkoxycarbonyl, C₁-C₆ alkylamino or C₂-C₆ dialkylamino.
Embodiment 138. A compound of Embodiment 137 wherein each R¹³ is independently halogen, cyano, C₁-C₃ alkyl, C₁-C₃ haloalkyl, C₂-C₄ alkenyl, C₂-C₄ haloalkenyl, C₃-C₆ cycloalkyl, C₂-C₄ alkoxyalkyl, C₂-C₄ haloalkoxyalkyl, C₁-C₃ alkoxy, C₁-C₃ haloalkoxy, C₂-C₄ alkenyloxy, C₂-C₄ haloalkenyloxy, C₁-C₃ alkylthio, C₁-C₃ haloalkylthio, C₂-C₄ alkylcarbonyl, C₂-C₄ haloalkylcarbonyl.
Embodiment 139. A compound of Embodiment 138 wherein each R¹³ is independently halogen, C₁-C₃ alkyl, C₁-C₃ haloalkyl, C₂-C₄ alkenyl, C₂-C₄ haloalkenyl, C₁-C₃ alkoxy, C₁-C₃ haloalkoxy, C₁-C₃ alkylthio or C₁-C₃ haloalkylthio.
Embodiment 140. A compound of Embodiment 139 wherein each R¹³ is independently halogen, C₁-C₃ alkyl, C₁-C₃ haloalkyl, C₁-C₃ alkoxy or C₁-C₃ haloalkoxy.
Embodiment 141. A compound of Embodiment 140 wherein each R¹³ is independently halogen, methyl, trifluoromethyl or methoxy.
Embodiment 142. A compound of Embodiment 141 wherein each R¹³ is independently halogen.
Embodiment 143. A compound of Formula 1 or any one of Embodiments 1 through 127 wherein each R^{14a} is independently H, cyano, hydroxy, C₁-C₄ alkyl, C₁-C₄ haloalkyl, C₂-C₄ alkenyl, C₂-C₄ haloalkenyl, C₂-C₄ alkoxyalkyl, C₁-C₄ alkylsulfonyl, C₂-C₄ alkylthioalkyl, C₂-C₄ alkylsulfonylalkyl, C₂-C₄ alkylcarbonyl, C₂-C₄ haloalkylcarbonyl, C₂-C₄ alkoxycarbonyl, C₃-C₅ alkoxycarbonylalkyl, C₂-C₄ alkylaminocarbonyl or C₃-C₅ dialkylaminocarbonyl.
Embodiment 144. A compound of Embodiment 143 wherein each R^{14a} is independently H, cyano, C₁-C₄ alkyl, C₁-C₄ haloalkyl, C₂-C₄ alkenyl, C₂-C₄ haloalkenyl, C₂-C₄ alkoxyalkyl, C₂-C₄ alkylcarbonyl, C₂-C₄ haloalkylcarbonyl or C₂-C₄ alkoxycarbonyl.
Embodiment 145. A compound of Embodiment 144 wherein each R^{14a} is independently H, C₁-C₂ alkyl, C₁-C₂ haloalkyl, C₂-C₄ alkoxyalkyl or C₂-C₄ alkylcarbonyl.
Embodiment 146. A compound of Embodiment 145 wherein each R^{14a} is independently H, methyl, trifluoromethyl or methoxymethyl.
Embodiment 147. A compound of Embodiment 146 wherein each R^{14a} is independently H or methyl.
Embodiment 148. A compound of Formula 1 or any one of Embodiments 1 through 147 wherein each R^{14b} is independently H, C₁-C₃ alkyl, C₁-C₃ haloalkyl, C₂-C₄ alkenyl, C₂-C₄ haloalkenyl, C₁-C₃ hydroxyalkyl, C₂-C₄ cyanoalkyl, C₃-C₆ cycloalkyl, C₃-C₆ halocycloalkyl, C₄-C₁₀ alkylcycloalkyl, C₄-C₁₀ cycloalkylalkyl, C₂-C₄ alkoxyalkyl, C₂-C₄ haloalkoxyalkyl, C₂-C₄ alkylaminoalkyl or C₂-C₄ haloalkylaminoalkyl.
Embodiment 149. A compound of Embodiment 148 wherein each R^{14b} is independently H, C₁-C₃ alkyl, C₁-C₃ haloalkyl, C₂-C₄ alkenyl, C₂-C₄ haloalkenyl, C₃-C₆ cycloalkyl, C₃-C₆ halocycloalkyl, C₄-C₁₀ cycloalkylalkyl, C₂-C₄ alkoxyalkyl or C₂-C₄ alkylaminoalkyl.
Embodiment 150. A compound of Embodiment 149 wherein each R^{14b} is independently H, methyl, trifluoromethyl, cyclopropyl or methoxymethyl.
Embodiment 151. A compound of Embodiment 150 wherein each R^{14b} is independently H or methyl.
Embodiment 152. A compound of Formula 1 or any one of Embodiments 1 through 151 wherein each R¹⁵ is independently cyano, halogen, hydroxy, C₁-C₄ alkyl, C₁-C₄ haloalkyl, C₃-C₆ cycloalkyl, C₃-C₆ halocycloalkyl, C₁-C₄ alkoxy, C₁-C₄ haloalkoxy, C₁-C₄ alkylthio, C₁-C₄ alkylsulfonyl, C₂-C₄ alkylcarbonyl, C₂-C₅ alkoxycarbonyl, C₁-C₄ alkylamino or C₂-C₅ dialkylamino.
Embodiment 153. A compound of Embodiment 152 wherein each R¹⁵ is independently, C₁-C₃ alkyl, C₁-C₃ haloalkyl, cyclopropyl, C₁-C₃ alkoxy, C₁-C₃ haloalkoxy, C₂-C₄ alkylcarbonyl or C₂-C₅ alkoxycarbonyl.
Embodiment 154. A compound of Embodiment 153 wherein each R¹⁵ is independently, trifluoromethyl, cyclopropyl, methoxy, halomethoxy, methylcarbonyl or methoxycarbonyl.
Embodiment 155. A compound of Formula 1 or any one of Embodiments 1 through 154 wherein each U is independently a direct bond, C(═O)O or C(═O)N(R¹⁸).
Embodiment 156. A compound of Embodiment 155 wherein each U is a direct bond.
Embodiment 157. A compound of Embodiment 155 wherein each U is independently C(═O)O or C(═O)N(R¹⁸).
Embodiment 158. A compound of Formula 1 or any one of Embodiments 1 through 157 wherein each V is independently a direct bond; or C₁-C₃ alkylene, C₂-C₄ alkenylene or C₃-C₄ alkynylene, each optionally substituted with up to 2 substituents independently selected from halogen, hydroxy, C₁-C₂ alkyl, C₁-C₂ alkoxy and C₁-C₂ haloalkoxy.
Embodiment 159. A compound of Embodiment 158 wherein each V is independently a direct bond, C₁-C₃ alkylene, C₂-C₄ alkenylene or C₃-C₄ alkynylene.
Embodiment 160. A compound of Embodiment 159 wherein each V is independently C₁-C₃ alkylene.
Embodiment 161. A compound of Formula 1 or any one of Embodiments 1 through 160 wherein each T is independently phenyl optionally substituted with up to 2 substituents independently selected from R²⁰; or a 5- to 6-membered heteroaromatic ring, each ring containing ring members selected from carbon atoms and 1 to 4 heteroatoms independently selected from up to 2 O, up to 2 S and up to 4 N atoms, each ring optionally substituted with up to 2 substituents independently selected from R²⁰; or a 3- to 7-membered nonaromatic heterocyclic ring, each ring containing ring members selected from carbon atoms and 1 to 4 heteroatoms independently selected from up to 2 O, up to 2 S and up to 4 N atoms, wherein up to 2 ring members are independently selected from C(═O), C(═S), S(═O) and S(═O)₂, each ring optionally substituted with up to 2 substituents independently selected from R²⁰.
Embodiment 162. A compound of Embodiment 161 wherein each T is independently phenyl, pyridinyl, pyrazolyl, imidazolyl, triazolyl, thiazolyl, oxazolyl, isoxazolyl, thienyl, isoxazolinyl, piperidinyl, morpholinyl or piperazinyl, each optionally substituted with up to 2 substituents independently selected from R²⁰.
Embodiment 163. A compound of Embodiment 162 wherein each T is independently phenyl, pyridinyl, pyrazolyl, imidazolyl, triazolyl, thiazolyl or oxazolyl, each optionally substituted with up to 2 substituents independently selected from R²⁰.
Embodiment 164. A compound of Embodiment 163 wherein each T is independently phenyl, pyridinyl or pyrazolyl, each optionally substituted with up to 2 substituents independently selected from R²⁰.
Embodiment 165. A compound of Formula 1 or any one of Embodiments 1 through 164 wherein each R¹⁶ is independently halogen, cyano, C₁-C₃ alkyl, C₁-C₃ haloalkyl, C₁-C₃ alkoxy, C₁-C₃ haloalkoxy, C₁-C₃ alkylthio, C₁-C₃ alkylsulfonyl or C₁-C₃ haloalkylsulfonyl.
Embodiment 166. A compound of Embodiment 165 wherein each R¹⁶ is independently halogen, C₁-C₃ alkyl, C₁-C₃ haloalkyl, C₁-C₃ alkoxy or C₁-C₃ haloalkoxy.
Embodiment 167. A compound of Embodiment 166 wherein each R¹⁶ is independently halogen, methyl or trifluoromethyl.
Embodiment 168. A compound of Formula 1 or any one of Embodiments 1 through 167 wherein each R¹⁷ is independently halogen, cyano, C₁-C₃ alkyl, C₁-C₃ haloalkyl, C₁-C₃ alkoxy or C₁-C₃ haloalkoxy.
Embodiment 169. A compound of Embodiment 168 wherein each R¹⁷ is independently halogen, C₁-C₃ alkyl or C₁-C₃ haloalkyl.
Embodiment 170. A compound of Embodiment 169 wherein each R¹⁷ is independently halogen, methyl or halomethyl.
Embodiment 171. A compound of Formula 1 or any one of Embodiments 1 through 170 wherein each R¹⁸ and R¹⁹ is independently H, cyano, hydroxy, C₁-C₄ alkyl or C₁-C₄ haloalkyl.
Embodiment 172. A compound of Embodiment 171 wherein each R¹⁸ and R¹⁹ is independently H, cyano, hydroxy or C₁-C₂ alkyl.
Embodiment 173. A compound of Formula 1 or any one of Embodiments 1 through 172 wherein each R²⁰ is independently halogen, cyano, C₁-C₄ alkyl, C₁-C₄ haloalkyl or C₁-C₄ alkoxy.
Embodiment 174. A compound of Embodiment 173 wherein each R²⁰ is independently halogen, cyano, C₁-C₂ alkyl, C₁-C₂ haloalkyl or C₁-C₂ alkoxy.
Embodiment 175. A compound of Embodiment 174 wherein each R²⁰ is independently halogen, methyl or methoxy.
Embodiment 176. A compound of Formula 1 or any one of Embodiments 1 through 175 wherein when R^{21a} is separate (i.e. not taken together with R^{21b} to form a ring), then R^{21a} is H, methyl, halomethyl or C₂-C₄ alkylcarbonyl.
Embodiment 177. A compound of Embodiment 176 wherein R^{21a} is H, methyl or trifluoromethyl.
Embodiment 178. A compound of Embodiment 177 wherein R^{21a} is H or methyl.
Embodiment 179. A compound of Formula 1 or any one of Embodiments 1 through 178 wherein when R^{21b} is separate (i.e. not taken together with R^{21a} to form a ring), then R^{21b} is H, cyano, C₁-C₅ alkyl, C₁-C₅ haloalkyl, C₁-C₆ cycloalkyl, C₁-C₆ halocycloalkyl, C₂-C₅ alkylcarbonyl, C₂-C₅ haloalkylcarbonyl, C₂-C₅ alkoxycarbonyl, C₂-C₅ haloalkoxycarbonyl, C₃-C₅ alkoxycarbonylalkyl or C₂-C₅ alkylaminocarbonyl.
Embodiment 180. A compound of Embodiment 179 wherein R^{21b} is H, cyano, C₁-C₅ alkyl, C₁-C₅ haloalkyl, C₁-C₆ cycloalkyl, C₂-C₅ alkylcarbonyl, C₂-C₅ haloalkylcarbonyl, C₂-C₅ alkoxycarbonyl or C₂-C₅ haloalkoxycarbonyl.
Embodiment 181. A compound of Embodiment 180 wherein R^{21b} is H, cyano, C₁-C₃ alkyl, C₁-C₃ haloalkyl, cyclopropyl C₂-C₂ alkylcarbonyl or C₂-C₂ alkoxycarbonyl.
Embodiment 182. A compound of Embodiment 181 wherein R^{21b} is H, C₁-C₃ alkyl or C₁-C₃ haloalkyl.
Embodiment 183. A compound of Embodiment 182 wherein R^{21b} is H or methyl.
Embodiment 184. A compound of Formula 1 or any one of Embodiments 1 through 183 wherein when R^{21a} and R^{21b} are taken together to form a ring (i.e. R^{21a} and R^{21b} are not separate), then said ring is a 5- to 6-membered fully saturated heterocyclic ring, each ring containing ring members, in addition to the connecting nitrogen atom, selected from carbon atoms and up to 2 heteroatoms independently selected from up to 2 O, up to 2 S and up to 2 N atoms, each ring optionally substituted with up to 3 substituents independently selected from halogen, cyano, C₁-C₃ alkyl, C₁-C₃ haloalkyl, C₁-C₃ alkoxy and C₁-C₃ haloalkoxy.
Embodiment 185. A compound of Embodiment 184 wherein R^{21a} and R^{21b} are taken together to form a 5- to 6-membered fully saturated heterocyclic ring, each ring containing ring members, in addition to the connecting nitrogen atom, selected from carbon atoms and up to 2 heteroatoms independently selected from up to 2 O, up to 2 S and up to 2 N atoms, each ring optionally substituted with up to 3 substituents independently selected from halogen, cyano, methyl, halomethyl or methoxy.
Embodiment 186. A compound of Embodiment 185 wherein R^{21a} and R^{21b} are taken together to form a 5- to 6-membered fully saturated heterocyclic ring, each ring containing ring members, in addition to the connecting nitrogen atom, selected from carbon atoms and up to 2 heteroatoms independently selected from up to 1 O, up to 1 S and up to 2 N atoms, each ring optionally substituted with up to 3 substituents independently selected from halogen or methyl.

Embodiments of this invention, including Embodiments 1-186 above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this invention, including Embodiments 1-186 above as well as any other embodiments described herein, and any combination thereof, pertain to the compositions and methods of the present invention.

Combinations of Embodiments 1-186 are illustrated by:

Embodiment A. A compound of Formula 1 wherein
Q is selected from:

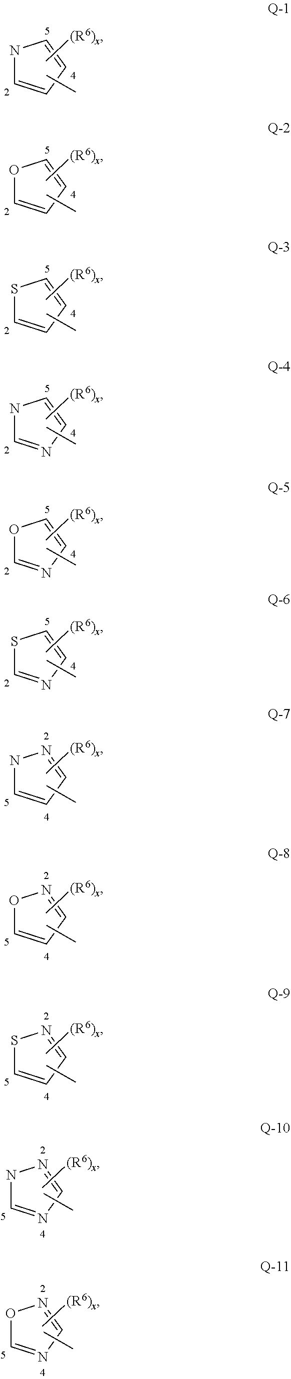

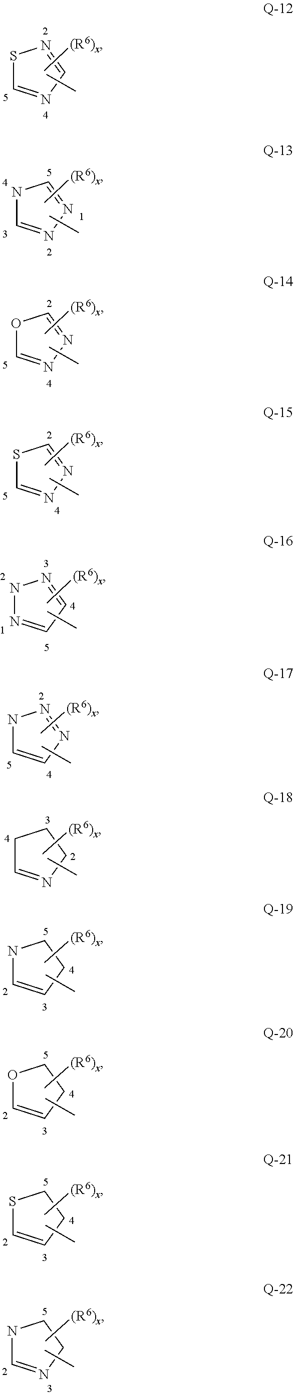

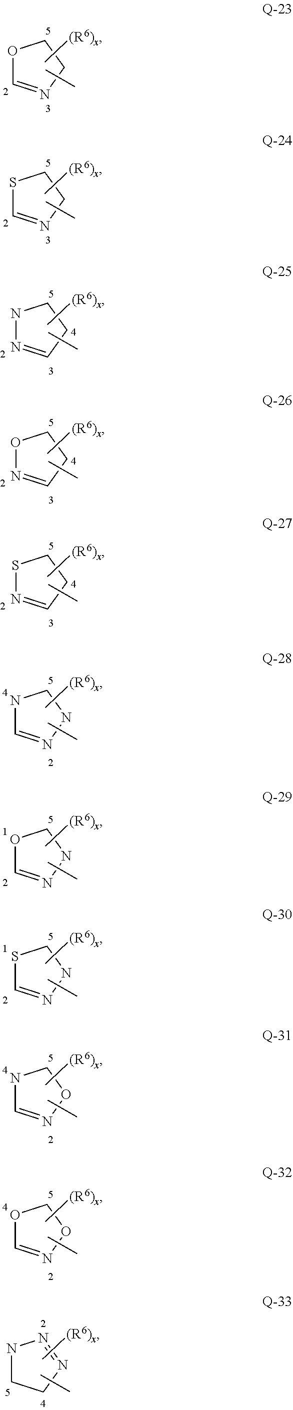

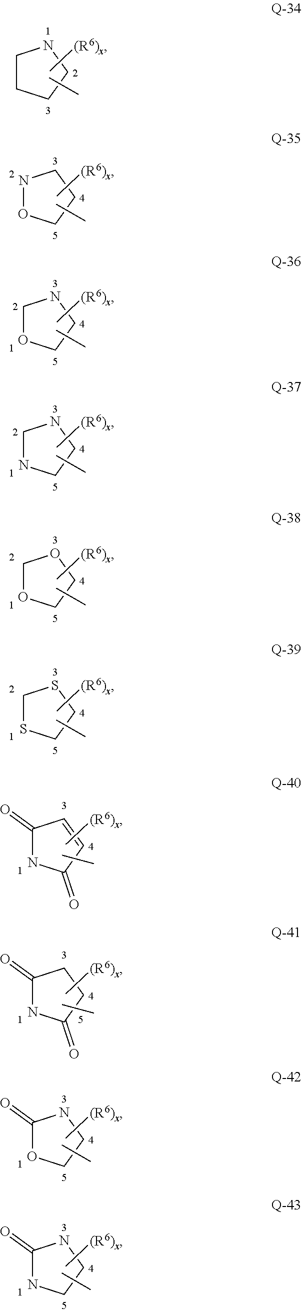

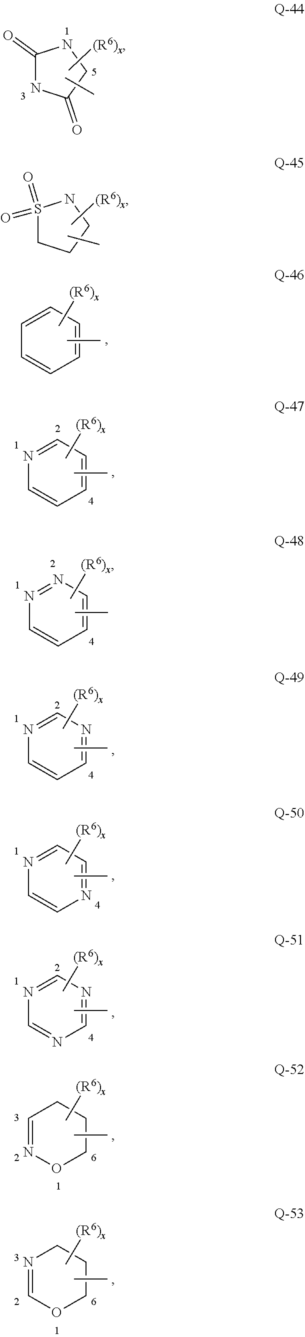

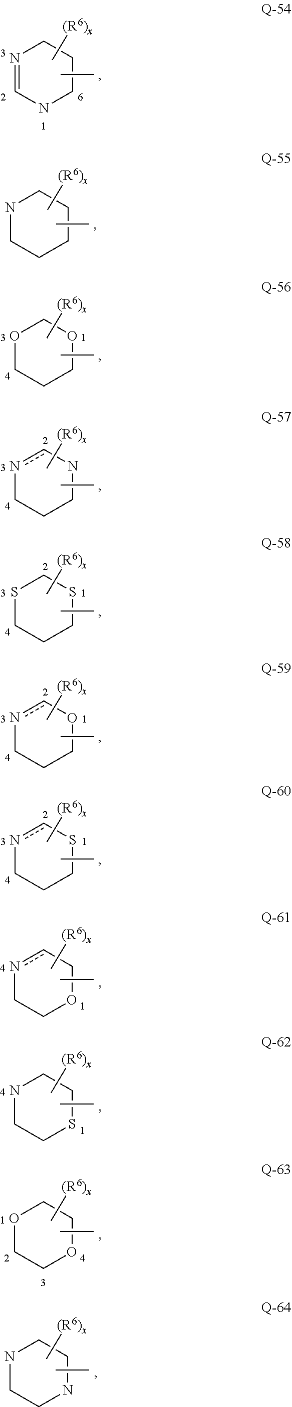

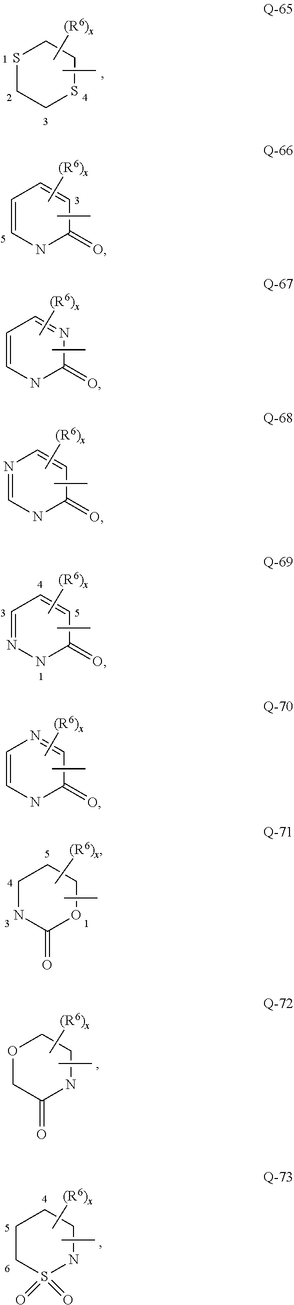

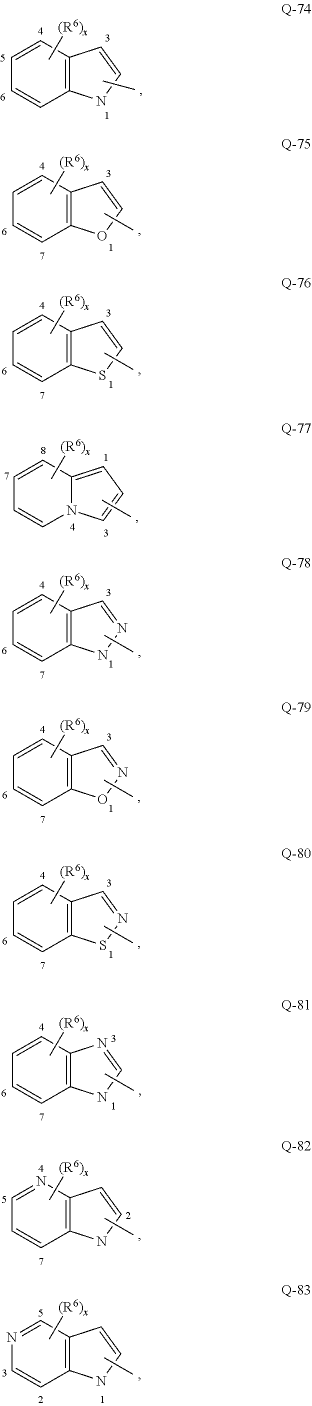

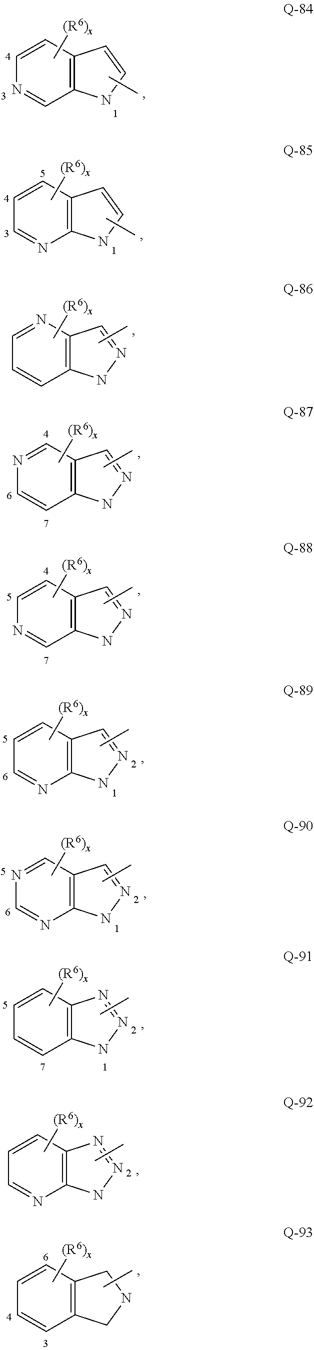

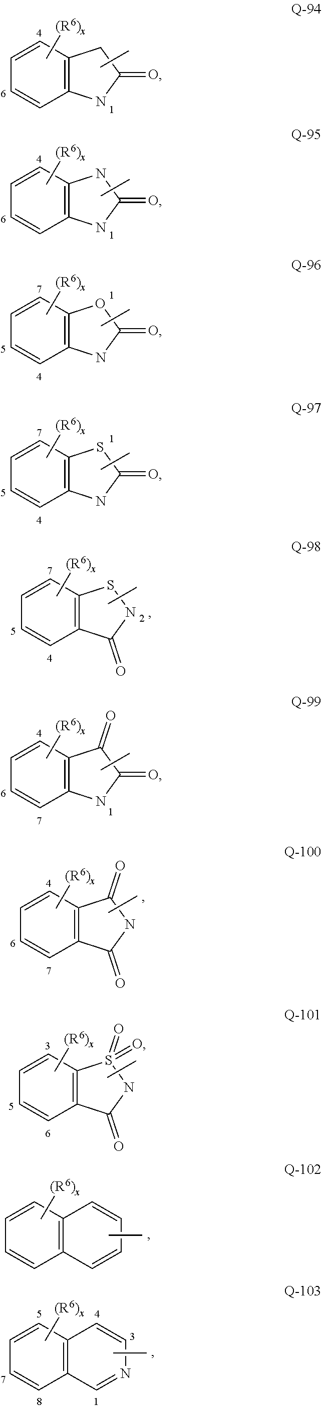

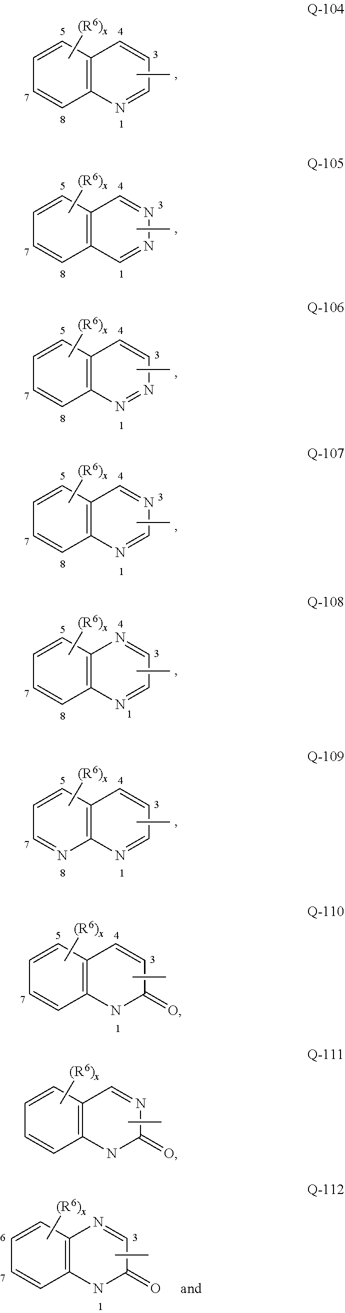

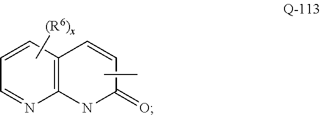

wherein the floating bond is connected to Formula 1 through any available carbon or nitrogen atom of the depicted ring or ring system; and x is 0, 1, 2 or 3;
Y is CR⁴ or N;
R^{1a} is CF₃, CCl₃ or CF₂C₁
R^{1b} is H, halogen, hydroxy, methyl, halomethyl, methoxy or halomethoxy;
is CR^{7a}R^{7b};
each W is O;
R² is H or methyl;
R³ is H, halogen, C₁-C₃ alkyl, C₁-C₃ haloalkyl or OR⁸;
R⁴ is halogen, cyano or C₁-C₂ alkyl;
m is 0 or 1;
R^{5a} is H, C₁-C₆ alkyl or C₁-C₆ haloalkyl;
R^{5b} is H, C₁-C₆ alkyl, C₁-C₆ haloalkyl, C₂-C₆ cyanoalkyl, C₃-C₆ cycloalkyl or C₄-C₁₀ cycloalkylalkyl; or a phenyl ring optionally substituted with up to 3 substituents independently selected from R⁹; or a 5- to 6-membered heterocyclic ring, each ring containing ring members selected from carbon atoms and 1 to 4 heteroatoms independently selected from up to 2 O, up to 2 S and up to 4 N atoms, wherein up to 2 ring members are independently selected from C(═O), C(═S), S(═O) and S(═O)₂, each ring optionally substituted with up to 3 substituents independently selected from R⁹; or -A(CR^{10a}R^{10b})ₙB or NR^{21a}R^{21b}; or
R^{5a} and R^{5b} are taken together to form a 5- to 6-membered fully saturated heterocyclic ring, each ring containing ring members, in addition to the connecting nitrogen atom, selected from carbon atoms and up to 2 heteroatoms independently selected from up to 2 O, up to 2 S and up to 2 N atoms, each ring optionally substituted with up to 3 substituents independently selected from R¹³;
A is 0 or direct bond;
n is 0 or 1;
B is a phenyl ring optionally substituted with up to 3 substituents independently selected from R¹²;
each R⁶ is independently cyano, halogen, nitro, NR^{14a}R^{14b}, C(═O)NR^{14a}R^{14b}, C(═S)NR^{14a}R^{14b} or —U—V-T; or C₁-C₆ alkyl, C₁-C₆ haloalkyl, C₂-C₆ alkenyl, C₂-C₆ haloalkenyl, C₃-C₆ cycloalkyl, C₃-C₆ halocycloalkyl, C₂-C₆ cyanoalkyl, C₄-C₁₀ alkylcycloalkyl, C₄-C₁₀ cycloalkylalkyl, C₁-C₆ alkoxyalkyl, C₁-C₆ alkylthioalkyl, C₁-C₆ alkoxy, C₁-C₆ haloalkoxy, C₂-C₆ alkenyloxy, C₂-C₆ haloalkenyloxy, C₂-C₆ alkoxyalkoxy, C₁-C₆ alkylthio, C₁-C₆ alkylamino, C₂-C₆ dialkylamino or C₁-C₆ haloalkylamino, each optionally substituted with up to 3 substituents independently selected from R¹⁵;
R^{7a} is H, C₁-C₃ alkyl or C₁-C₃ haloalkyl;
R^{7b} is H, C₁-C₃ alkyl, C₁-C₃ haloalkyl, C₃-C₆ cycloalkyl, C₃-C₆ halocycloalkyl, C₂-C₃ alkoxyalkyl; or phenyl optionally substituted with up to 3 substituents independently selected from R¹⁶;
R^{7c} is H, methyl, trifluoromethyl or C₂-C₄ alkylcarbonyl;
R⁸ is H, C₁-C₃ alkyl or C₁-C₃ haloalkyl; or phenyl optionally substituted with up to 3 substituents independently selected from R¹⁷;
each R⁹ is independently cyano, halogen, nitro, C₁-C₃ alkyl, C₁-C₃ haloalkyl, C₁-C₃ alkoxy, C₁-C₃ haloalkoxy, C₂-C₄ alkoxyalkoxy, C₁-C₃ alkylthio, C₂-C₄ alkylcarbonyl, C₂-C₄ alkoxycarbonyl, C₁-C₃ alkylamino or C₂-C₄ dialkylamino;
R^{10a} is H, Br, Cl, F, cyano or methyl;
R^{10b} is H or methyl;
each R¹² is independently halogen, methyl, halomethyl or methoxy;
each R¹³ is independently halogen, cyano, C₁-C₃ alkyl, C₁-C₃ haloalkyl, C₂-C₄ alkenyl, C₂-C₄ haloalkenyl, C₃-C₆ cycloalkyl, C₂-C₄ alkoxyalkyl, C₂-C₄ haloalkoxyalkyl, C₁-C₃ alkoxy, C₁-C₃ haloalkoxy, C₂-C₄ alkenyloxy, C₂-C₄ haloalkenyloxy, C₁-C₃ alkylthio, C₁-C₃ haloalkylthio, C₂-C₄ alkylcarbonyl, C₂-C₄ haloalkylcarbonyl; each R^{14a} is independently H, C₁-C₂ alkyl, C₁-C₂ haloalkyl, C₂-C₄ alkoxyalkyl or C₂-C₄ alkylcarbonyl;
each R^{14b} is independently H, C₁-C₃ alkyl, C₁-C₃ haloalkyl, C₂-C₄ alkenyl, C₂-C₄ haloalkenyl, C₃-C₆ cycloalkyl, C₃-C₆ halocycloalkyl, C₄-C₁₀ cycloalkylalkyl, C₂-C₄ alkoxyalkyl or C₂-C₄ alkylaminoalkyl;
each R¹⁵ is independently, C₁-C₃ alkyl, C₁-C₃ haloalkyl, cyclopropyl, C₁-C₃ alkoxy, C₁-C₃ haloalkoxy, C₂-C₄ alkylcarbonyl or C₂-C₅ alkoxycarbonyl;
each U is independently direct bond, C(═O)O or C(═O)N(R¹⁸);
each V is independently a direct bond, C₁-C₃ alkylene, C₂-C₄ alkenylene or C₃-C₄ alkynylene;
each T is independently phenyl, pyridinyl or pyrazolyl, each optionally substituted with up to 2 substituents independently selected from R²⁰;
each R¹⁶ is independently halogen, C₁-C₃ alkyl, C₁-C₃ haloalkyl, C₁-C₃ alkoxy or C₁-C₃ haloalkoxy;
each R¹⁷ is independently halogen, cyano, C₁-C₃ alkyl, C₁-C₃ haloalkyl, C₁-C₃ alkoxy or C₁-C₃ haloalkoxy;
each R¹⁸ is independently H, cyano, hydroxy or C₁-C₂ alkyl;
each R²⁰ is independently halogen, cyano, C₁-C₂ alkyl, C₁-C₂ haloalkyl or C₁-C₂ alkoxy; and
R^{21a} is H, methyl, halomethyl or C₂-C₄ alkylcarbonyl;
R^{21b} is H, cyano, C₁-C₅ alkyl, C₁-C₅ haloalkyl, C₁-C₆ cycloalkyl, C₁-C₆ halocycloalkyl, C₂-C₅ alkylcarbonyl, C₂-C₅ haloalkylcarbonyl, C₂-C₅ alkoxycarbonyl, C₂-C₅ haloalkoxycarbonyl, C₃-C₅ alkoxycarbonylalkyl or C₂-C₅ alkylaminocarbonyl; or
R^{21a} and R^{21b} are taken together to form a 5- to 6-membered fully saturated heterocyclic ring, each ring containing ring members, in addition to the connecting nitrogen atom, selected from carbon atoms and up to 2 heteroatoms independently selected from up to 2 O, up to 2 S and up to 2 N atoms, each ring optionally substituted with up to 3 substituents independently selected from halogen, cyano, methyl, halomethyl or methoxy.
   Embodiment B. A compound of Embodiment A wherein
Q is Q-46 through Q-50;
R^{1a} is CF₃;
R^{1b} is H, Br, Cl, F, hydroxy, methyl, halomethyl, methoxy or halomethoxy;
is CR^{7a}R^{7b};
R² is H; or C₁-C₃ alkyl, C₂-C₃ alkenyl, C₂-C₃ alkylcarbonyl or C₂-C₃ alkoxycarbonyl, each optionally substituted with up to 3 substituents independently selected from Br, Cl, F or methyl;
R³ is H or halogen;
R⁴ is Br, Cl, F or methyl;
R^{5a} is H, C₁-C₃ alkyl or C₁-C₃ haloalkyl;
R^{5b} is H, C₁-C₆ haloalkyl or C₃-C₆ cycloalkyl; or a phenyl ring optionally substituted with up to 3 substituents independently selected from R⁹; or a 6-membered heterocyclic ring, containing ring members selected from carbon atoms and 1 to 2 heteroatoms independently selected from up to 2 O, up to 2 S and up to 2 N atoms, each ring optionally substituted with up to 3 substituents independently selected from R⁹; or
R^{5a} and R^{5b} are taken together to form a 5- to 6-membered fully saturated heterocyclic ring, each ring containing ring members, in addition to the connecting nitrogen atom, selected from carbon atoms and up to 2 heteroatoms independently selected from up to 2 O, up to 2 S and up to 2 N atoms, each ring optionally substituted with up to 3 substituents independently selected from R¹³;
each R⁶ is halogen, C₁-C₃ alkyl, C₁-C₃ haloalkyl or C₁-C₃ alkoxy;
R^{7a} is H, methyl or trifluoromethyl;
R^{7b} is H, C₁-C₃ alkyl or C₁-C₃ haloalkyl;
each R⁹ is independently cyano, halogen, nitro, C₁-C₃ alkyl, C₁-C₃ haloalkyl, C₁-C₃ alkoxy or C₁-C₃ haloalkoxy; and
each R¹³ is independently halogen, C₁-C₃ alkyl, C₁-C₃ haloalkyl, C₂-C₄ alkenyl, C₂-C₄ haloalkenyl, C₁-C₃ alkoxy, C₁-C₃ haloalkoxy, C₁-C₃ alkylthio or C₁-C₃ haloalkylthio.
   Embodiment C. A compound of Embodiment B wherein
Q is Q-46 or Q-47;
x is 1 or 2;
R^{1b} is H, Br, Cl, F, hydroxy or methyl;
R² is H or methyl;
R³ is halogen;
R⁴ is Br Cl or F;
R^{5a} is H or methyl;
R^{5b} is C₁-C₆ haloalkyl or C₁-C₆ cycloalkyl; or a phenyl or pyridinyl ring, each ring optionally substituted with up to 3 substituents independently selected from R⁹; each R⁶ is independently Br, Cl, F or trifluoromethyl;
R^{7a} is H;
R^{7b} is H or methyl; and
each R⁹ is independently halogen, methyl, trifluoromethyl or methoxy.
   Embodiment D. A compound of Embodiment C wherein
Q is Q-46;
R^{1b} is H;
R² is H;
m is 0;
R^{5a} is H;
R^{5b} is C₁-C₃ fluoroalkyl or cyclopropyl; or a phenyl or pyridinyl ring, each ring optionally substituted with up to 3 substituents independently selected from R⁹;
each R⁶ is independently Cl, F or trifluoromethyl;
R^{7b} is H; and
each R⁹ is independently Cl, F or trifluoromethyl.
   Embodiment E. A compound of Embodiment D wherein
R^{5b} is cyclopropyl; or a phenyl, 2-pyridinyl or 3-pyridinyl ring, each ring optionally substituted with up to 3 substituents independently selected from R⁹.
   Embodiment F. A compound of Embodiment E wherein
Q is Q-46 substituted at the 3- and 4-positions with substituents independently selected from R⁶; or Q is Q-46 substituted at the 3- and 5-positions with substituents independently selected from R⁶; or Q is Q-46 substituted at the 3-position with a substituent selected from R⁶; and
R^{5b} is a phenyl ring substituted at the 2-, 4- and 6-positions with substituents independently selected from R⁹; or a phenyl ring substituted at the 2- and 4-positions with substituents independently selected from R⁹; or a phenyl ring substituted at the 4-position with a substituent selected from R⁹.

Specific embodiments include compounds of Formula 1 selected from the group consisting of:
3,4-dichloro-N-[4-chloro-3-[[(4-fluorophenyl)amino]carbonyl]phenyl]-β-(trifluoro-methyl)benzenepropanamide (Compound 1);
N-[4-chloro-3-[[(2,4-difluorophenyl)amino]carbonyl]phenyl]-β,3-bis(trifluoromethyl)-benzenepropanamide (Compound 5);
N-[4-chloro-3-[[[4-(trifluoromethyl)phenyl]amino]carbonyl]phenyl]-β,3-bis(trifluoro-methyl)benzenepropanamide (Compound 8);
3,5-dichloro-N-[4-chloro-3-[[(3-fluorophenyl)amino]carbonyl]phenyl]-β-(trifluoro-methyl)benzenepropanamide (Compound 32);
3,4-dichloro-N-[4-chloro-3-[[(2,4-difluorophenyl)amino]carbonyl]phenyl]-β-(trifluoro-methyl)benzenepropanamide (Compound 52);
3,5-dichloro-N-[4-chloro-3-[[(2,4-difluorophenyl)amino]carbonyl]phenyl]-β-(trifluoro-methyl)benzenepropanamide (Compound 60);
3,5-dichloro-N-[4-chloro-3-[[(4-fluorophenyl)amino]carbonyl]phenyl]-β-(trifluoro-methyl)benzenepropanamide (Compound 76);
N-[4-chloro-3-[[(2-chloro-4-fluorophenyl)amino]carbonyl]phenyl]-β,3-bis(trifluoromethyl)-benzenepropanamide (Compound 82);
N-[4-chloro-3-[[(4-chloro-2-fluorophenyl)amino]carbonyl]phenyl]-β,3-bis(trifluoro-methyl)benzenepropanamide (Compound 83);
N-[4-chloro-3-[[(2,4,6-trifluorophenyl)amino]carbonyl]phenyl]-β,3-bis(trifluoromethyl)-benzenepropanamide (Compound 85);
3,5-dichloro-N-[4-chloro-3-[[(2,4-difluorophenyl)amino]carbonyl]phenyl]-4-fluoro-β-(trifluoromethyl)benzenepropanamide (Compound 105);
3-chloro-N-[4-chloro-3-[[(2,4-difluorophenyl)amino]carbonyl]phenyl]-4-fluoro-β-(trifluoromethyl)benzenepropanamide (Compound 115);
(βR)—N-[4-chloro-3-[[(2,4-difluorophenyl)amino]carbonyl]phenyl]-β,3-bis(trisfluoromethyl)benzenepropanamide (Compound 103);
N-[4-chloro-3-[[(2,4-difluorophenyl)amino]carbonyl]phenyl]-N-methyl-β,3-bis(trifluoromethyl)benzenepropanamide (Compound 145);
N-[4-chloro-3-[[(2,4-difluorophenyl)amino]carbonyl]phenyl]-β,3,5-tris(trifluoromethyl) benzenepropanamide (Compound 142);
N-[4-chloro-3-[[(2,4-difluorophenyl)methylamino]carbonyl]phenyl]-β,3-bis(trifluoromethyl)benzenepropanamide (Compound 148);

Of note is that compounds of this invention are characterized by favorable metabolic and/or soil residual patterns and exhibit activity controlling a spectrum of agronomic and nonagronomic invertebrate pests.

Of particular note, for reasons of invertebrate pest control spectrum and economic importance, protection of agronomic crops from damage or injury caused by invertebrate pests by controlling invertebrate pests are embodiments of the invention. Compounds of this invention because of their favorable translocation properties or systemicity in plants also protect foliar or other plant parts which are not directly contacted with a compound of Formula 1 or a composition comprising the compound.

Also noteworthy as embodiments of the present invention are compositions comprising a compound of any of the preceding Embodiments, as well as any other embodiments described herein, and any combinations thereof, and at least one additional component selected from the group consisting of a surfactant, a solid diluent and a liquid diluent, said compositions optionally further comprising at least one additional biologically active compound or agent.

Further noteworthy as embodiments of the present invention are compositions for controlling an invertebrate pest comprising a compound of any of the preceding Embodiments, as well as any other embodiments described herein, and any combinations thereof, and at least one additional component selected from the group consisting of a surfactant, a solid diluent and a liquid diluent, said compositions optionally further comprising at least one additional biologically active compound or agent. Embodiments of the invention further include methods for controlling an invertebrate pest comprising contacting the invertebrate pest or its environment with a biologically effective amount of a compound of any of the preceding Embodiments (e.g., as a composition described herein).

Embodiments of the invention also include a composition comprising a compound of any of the preceding Embodiments, in the form of a soil drench liquid formulation. Embodiments of the invention further include methods for controlling an invertebrate pest comprising contacting the soil with a liquid composition as a soil drench comprising a biologically effective amount of a compound of any of the preceding Embodiments.

Embodiments of the invention also include a spray composition for controlling an invertebrate pest comprising a biologically effective amount of a compound of any of the preceding Embodiments and a propellant. Embodiments of the invention further include a bait composition for controlling an invertebrate pest comprising a biologically effective amount of a compound of any of the preceding Embodiments, one or more food materials, optionally an attractant, and optionally a humectant. Embodiments of the invention also include a device for controlling an invertebrate pest comprising said bait composition and a housing adapted to receive said bait composition, wherein the housing has at least one opening sized to permit the invertebrate pest to pass through the opening so the invertebrate pest can gain access to said bait composition from a location outside the housing, and wherein the housing is further adapted to be placed in or near a locus of potential or known activity for the invertebrate pest.

Embodiments of the invention also include methods for protecting a seed from an invertebrate pest comprising contacting the seed with a biologically effective amount of a compound of any of the preceding Embodiments.

Embodiments of the invention also include methods for protecting an animal from an invertebrate parasitic pest comprising administering to the animal a parasiticidally effective amount of a compound of any of the preceding Embodiments.

Embodiments of the invention also include methods for controlling an invertebrate pest comprising contacting the invertebrate pest or its environment with a biologically effective amount of a compound of Formula 1, an N-oxide or a salt thereof, (e.g., as a composition described herein), provided that the methods are not methods of medical treatment of a human or animal body by therapy.

This invention also relates to such methods wherein the invertebrate pest or its environment is contacted with a composition comprising a biologically effective amount of a compound of Formula 1, an N-oxide or a salt thereof, and at least one additional component selected from the group consisting of surfactants, solid diluents and liquid diluents, said composition optionally further comprising a biologically effective amount of at least one additional biologically active compound or agent, provided that the methods are not methods of medical treatment of a human or animal body by therapy.

Embodiments of this disclosure also include use of an unmanned aerial vehicle (UAV) for the dispersion of the compositions disclosed herein over a planted area. In some embodiments the planted area is a crop-containing area. In some embodiments, the crop is selected from a monocot or dicot. In some embodiments, the crop is selected form rice, corn, barley, soybean, wheat, vegetable, tobacco, tea tree, fruit tree and sugar cane. In some embodiments, the compositions disclosed herein are formulated for spraying at an ultra-low volume. Products applied by drones may use water or oil as the spray carrier. Typical spray volume (including product) used for drone applications globally. 5.0 liters/ha-100 liters/ha (approximately 0.5-10 gpa). This includes the range of ultra-low spray volume (ULV) to low spray volume (LV). Although not common there may be situations where even lower spray volumes could be used as low as 1.0 liter/ha (0.1 gpa).

One or more of the following methods and variations as described in Schemes 1-8 can be used to prepare the compounds of Formula 1. The definitions of Q, R^{1a}, R^{1b}, Z, W, R², R³, R⁴, m, R^{5a} and R^{5b} in the compounds of Formulae 1-9 below are as defined above in the Summary of the Invention unless otherwise noted. Compounds of Formula 1a are various subsets of the compounds of Formula 1, and all substituents for Formula 1a are as defined above for Formula 1 unless otherwise noted.

As shown in Scheme 1, compounds of Formula 1 (wherein Z is CR^{7a}R^{7b}) can be prepared by reacting a carboxylic acid of Formula 2 with an aniline of Formula 3 in the presence of a coupling reagent. Useful coupling reagents include, for example, O-(7-azabenzotriazol-1-yl)-N,N,N′,N′-tetramethyluronium hexafluorophosphate (HATU), 2-(1H-benzotriazol-1-yl)-1,1,3,3-tetramethyluronium hexafluorophosphate (HBTU) and propylphosphonic anhydride (T3P). Typically the reaction is run in a polar aprotic solvent such as N,N-dimethylformamide or tetrahydrofuran and in the presence of a base such pyridine, triethylamine or N,N-diisopropylethylamine. For reaction conditions useful in the method of Scheme 1, as well as other well-established coupling conditions see, for example, Journal of Organic Chemistry 2008, 73(7), 2731-2737; Tetrahedron Letters 2009, 50(45), 6200-6202; and Organic letters 2011, 13(12), 2988-91.

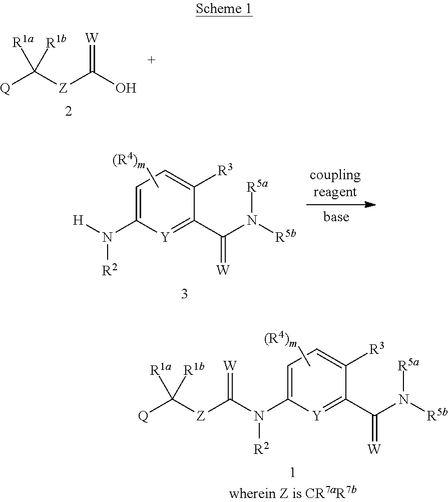

One skilled in the art will recognize that the method of Scheme 1 can also be practiced with chiral starting materials to obtain certain chiral compounds of Formula 1. For example, as shown in Scheme 2, the carboxylic acids of Formulae 2′ and 2″ (wherein Z is CR^{7a}R^{7b}) wherein R^{1a} is other than H and R^{1b} is H (i.e. the (R)-configured or (S)-configured carboxylic acids) can be reacted with an aniline of Formula 3 to provide the two enantiomers of Formula 1 shown below as Formula 1′ and Formula 1″ wherein the chiral center is identified with an asterisk (*).

Of note as starting materials in the method of Scheme 2 are compounds of Formulae 2′ and 2″ specifically disclosed in Tables 1 and 2 below.

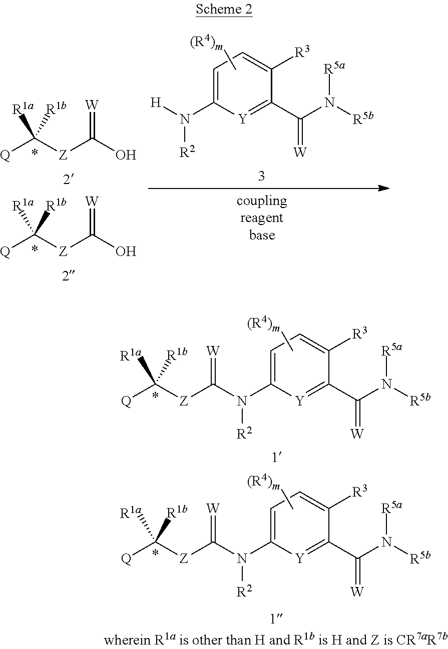

Scheme 3 illustrates an example of the general method of Scheme 1 for the preparation of a compound of Formula 1a (i.e. Formula 1 wherein Q is optionally substituted phenyl (i.e. Q-46), R^{1a} is CF₃, R^{1b} is H, Z is CH₂, W is O, and R² and R^{5a} are both H). In this method a compound of Formula 2a (i.e. Formula 2 wherein Q is optionally substituted phenyl, R^{1a} is CF₃. R^{1b} is H, Z is CH₂ and W is O) is reacted with an aniline of Formula 3a (i.e. Formula 3 wherein W is O, R² is H and R^{5a} is H) in the presence of HATU and triethylamine and in the solvent N,N-dimethylformamide. Present Example 1, Step D and Example 2, Step D illustrate the method of Scheme 3

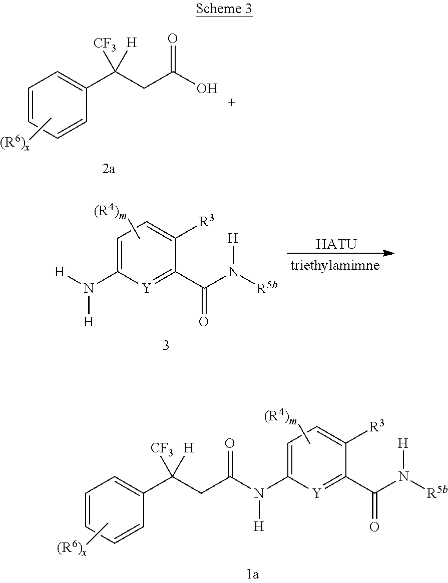

As shown in Scheme 4, compounds of Formula 1 can also be prepared by reacting an acid chloride of Formula 4 (wherein Z is CR^{7a}R^{7b}) with an aniline of Formula 3 in the presence of an acid scavenger. Typical acid scavengers include amine bases such as triethylamine, N,N-diisopropylethylamine and pyridine. Other scavengers include hydroxides such as sodium hydroxide and potassium hydroxide, or carbonates such as sodium carbonate and potassium carbonate. In certain instances it is useful to use polymer-supported acid scavengers such as polymer-bound N,N-diisopropylethylamine and polymer-bound 4-(dimethylamino)pyridine. One skilled in the art will recognize that mixtures may result when an aniline of Formula 3 contains a second NH function and standard methods of separation can be employed to isolate the desired compound.

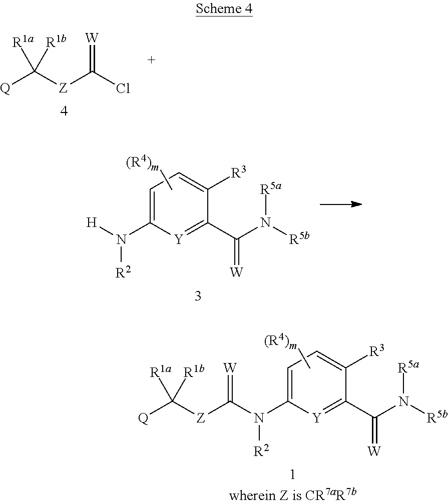

Acid chlorides of Formula 4 (wherein Z is CR^{7a}R^{7b}) wherein W is O are easily prepared from carboxylic acids of Formula 2 by numerous well-known methods. For example, reacting the carboxylic acid with a chlorinating reagent such as thionyl chloride, oxalyl chloride or phosphorus oxychloride in a solvent such as dichloromethane or toluene and optionally in the presence of a catalytic amount of N,N-dimethylformamide can provide the corresponding acid chloride of Formula 4.

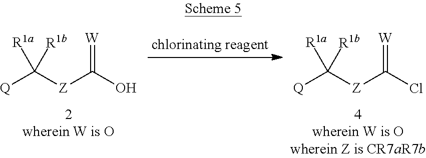

As shown in Scheme 6, carboxylic acids of Formula 2 wherein W is O and Z is CR^{7a}R^{7b} can be prepared according to well-known methods of basic or acidic hydrolysis of the corresponding compounds of Formula 5, preferably using a slight excess of potassium hydroxide or sodium hydroxide in a water-miscible co-solvent such as methanol, ethanol or tetrahydrofuran at a temperature between about 25 and 45° C. The product can be isolated by adjusting the pH to about 1 to 3 and then filtering or extracting, optionally after removal of the organic solvent by evaporation. Present Example 1, Step C and Example 2, Step C illustrate the method of Scheme 6.

Tables 1 and 2 below disclose specific compounds of Formula 2 which are particularly useful as intermediates in the methods of the present invention.

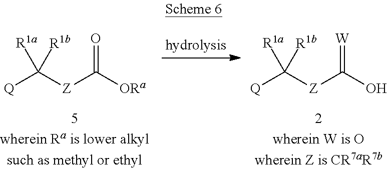

As outlined in Scheme 7, compounds of Formula 5 can be prepared from ketones of Formula 7 via either a Wittig reaction with a (triphenylphosphoranylidene)acetate of Formula 8 wherein Ra is typically methyl or ethyl in an inert solvent such as tetrahydrofuran or toluene, or by a Horner-Wadsworth-Emmons reaction using a diethoxyphosphonoacetic acid ester of Formula 9 wherein Ra is typically methyl or ethyl in the presence of a base such as sodium hydride or potassium tert-butoxide and in a suitable solvent that is generally anhydrous tetrahydrofuran or dioxane. Experimental conditions for a Wittig transformation are provided in PCT Patent Publication WO 2008/074752. Also, present Example 1, Step A and Example 2, Step A illustrate the preparation of a compound of Formula 6. In a subsequent step, compounds of Formula 6 can be reduced by catalytic hydrogenation. Typical conditions involve exposing a compound of Formula 6 to hydrogen gas at a pressure of 70 to 700 kPa, preferably 270 to 350 kPa, in the presence of a metal catalyst such as palladium supported on an inert carrier such as activated carbon, suspended in a solvent such as ethanol at ambient temperature. This type of reduction is very well-known; see, for example, Catalytic Hydrogenation, L. Cerveny, Ed., Elsevier Science, Amsterdam, 1986. For a representative procedure of the catalytic hydrogenation of Scheme 7, see WO 2009/012205. Also, the method of Scheme 7 is illustrated in Example 1, Step B and Example 2, Step B. One skilled in the art will recognize that other functionalities that may be present in compounds of Formula 6 can also be reduced under catalytic hydrogenation conditions, thus requiring a suitable choice of catalyst and conditions.

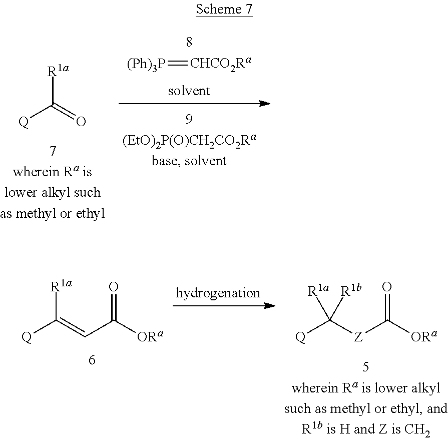

Compounds of Formula 6 are also useful intermediates for preparing certain chiral compounds of Formula 2, which in turn can be used to prepare chiral compounds of Formula 1 as described in Scheme 2. For example, as shown in Scheme 8, compounds of Formula 6 wherein R^{1a} is other than H can undergo asymmetric hydrogenation to access enantiomerically pure compounds of Formulae 5′ and 5″ wherein R^{5a} is other than H and R^{5b} is H (i.e. the (R)-configured or (k)-configured compounds). Subsequent hydrolysis provides the chiral compounds of Formulae 2a′ and 2a″ (i.e. Formulae 2′ and 2″ wherein W is O). For reviews of asymmetric catalytic hydrogenation methods including the use of ruthenium catalysts and ligands, see, for example, Accounts of Chemical Research 2003, 36(12), 908-918, and references cited therein; and Chemical Society Reviews 2013, 42(2), 728-754.

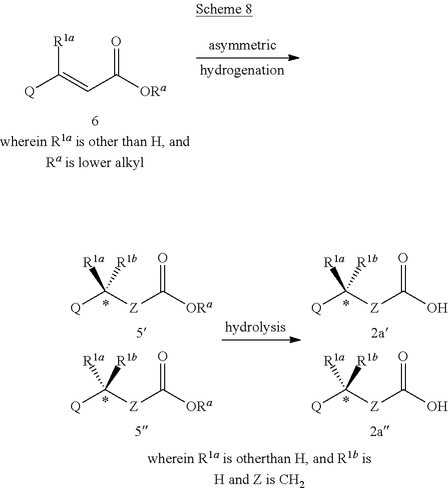

Chiral compounds of Formula 1 can also be obtained from a racemic mixture Formula 1 compounds through the utilization of well-known chiral chromatography separation methods. For extensive reviews of chiral separation methods see the Chiral Separations: Methods and Protocols (Methods in Molecular Biology), 2nd ed., 2013 Edition, by Gerhard K. E. Scriba (Editor).

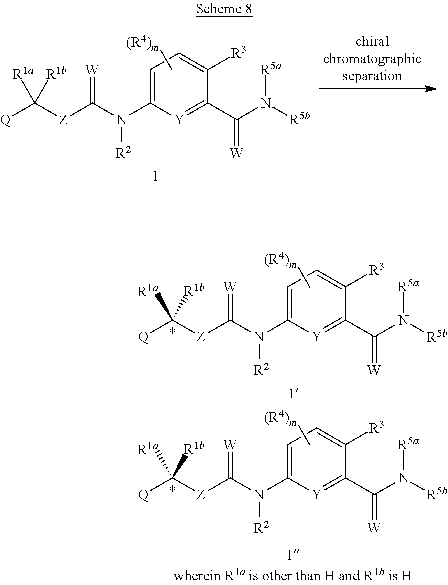

As shown in Scheme 9, compounds of Formula 1 in which Z is O and W is O may be prepared by reaction of a chloroformate 4a with an aniline 3. This reaction is carried out in an optional solvent or mixture of solvents such as toluene, dichloromethane, tetrahydrofuran, acetonitrile, ethyl acetate or water, optionally in the presence of an added base such as pyridine, triethylamine, potassium carbonate or sodium hydrogen carbonate, at temperatures typically from below ambient to ambient, or up to the boiling point of the solvent or solvent mixture. Preparation of chloroformates such as 4a and reactions with anilines are known; see for example Organic Letters 2000, 2(8), 1049-1051 and Journal of Medicinal Chemistry 2014, 57(15), 6704-6717.

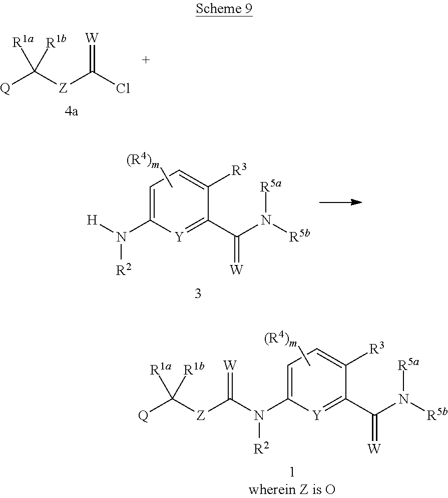

As shown in Scheme 10, compounds of Formula 1 wherein Z is O and W is O may alternatively be prepared by reaction of an alcohol (10) with an isocyanate (11). This reaction is carried out in an optional solvent or mixture of solvents such as toluene, tetrahydrofuran, dimethyl formamide or dichloromethane, optionally in the presence of an added base such as pyridine or sodium hydride, at temperatures from below ambient to the boiling point of the solvent or solvent mixture. One skilled in the art will select appropriate base and solvent combinations for chemical compatibility. For an example of this type of reaction, see Journal of Fluorine Chemistry 1991, 53(3), 327-338.

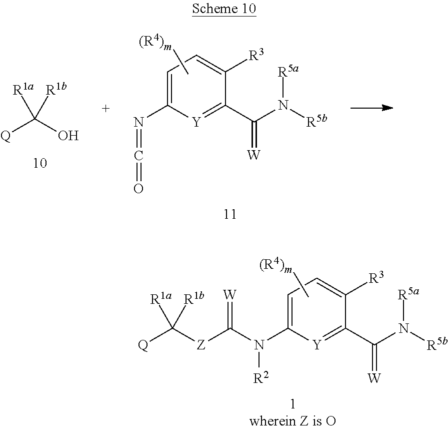

The alcohols (10) used in Scheme 10 and as starting materials for 4a in Scheme 9 are known or can be prepared by methods known in the art, and their preparation as single enantiomers is also known. For leading references, see for example Synlett 2020, 31(3), 237-247 and Chemistry—A European Journal 2019, 25(46), 10818-10822. The isocyanates are typically prepared from compounds 3 by reaction with phosgene or phosgene equivalents under conditions well-known to one skilled in the art. See for example, ACS Medicinal Chemistry Letters 2010. 1(9), 460-465 and Journal of the American Chemical Society 2006, 128(50), 16113-16121.
As shown in Scheme 11, compounds of Formula I in which Z is NR^{7c} and W is O can be prepared by reaction of an amine 12 with an isocyanate 11. This reaction is carried out in an optional solvent or mixture of solvents such as toluene, hexanes, tetrahydrofuran, dimethylformamide acetonitrile, or dichloromethane, optionally in the presence of an added base such as pyridine, triethylamine, potassium carbonate, or sodium hydride at temperatures from below ambient to the boiling point of the solvent or solvent mixture. One skilled in the art will select appropriate base and solvent combinations for chemical compatibility. For an example of this type of reaction, see Journal of Organic Chemistry, 2016, 81(8), 3263-3274 or Bioorganic and Medicinal Chemistry Letters, 2016, 26(5), 1386-1390. The amine 12 used in Scheme 11 can be prepared by methods known in the art and with high stereochemical purity. See for example Organic & Biomolecular Chemistry, 2019, 17(47), 10045-10051 and Journal of Organic Chemistry, 2016, 81(17), 7419-7431.

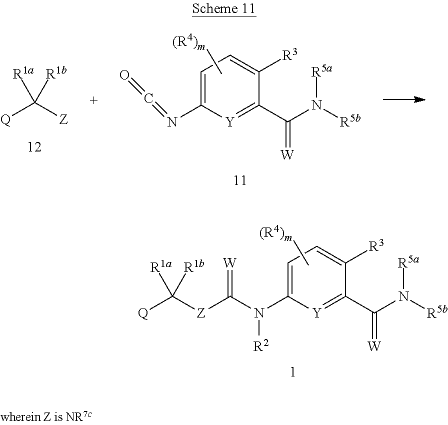

As shown in Scheme 12, compounds of Formula I wherein Z is NR^{7c} and W is O may also be prepared by the reaction of isocyanate 13 and aniline 3. This reaction is carried out in an optional solvent or mixture of solvents such as toluene, hexanes, tetrahydrofuran, dimethyl formamide acetonitrile, or dichloromethane, optionally in the presence of an added base such as pyridine, triethylamine, potassium carbonate, or sodium hydride at temperatures from below ambient to the boiling point of the solvent or solvent mixture. One skilled in the art will select appropriate base and solvent combinations for chemical compatibility. For an example of this type of reaction, see Journal of Organic Chemistry, 2019, 84(14), 8941-8947.

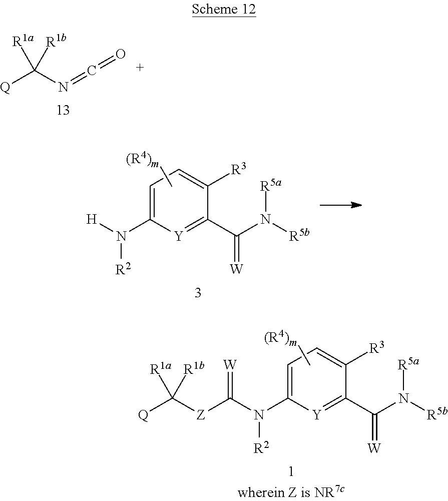

Isocyanates 11 (Scheme 11) and 13 (Scheme 12) can respectively be prepared by treating either an amine 12 or an aniline 3 (R²═H) with a phosgene equivalent. As an example, useful reagents include phosgene, triphosgene, 1,1′-carbonyldiimidazole (CDI), or a chloroformate. This reaction is carried out in an optional solvent or mixture of solvents such as toluene, dichloromethane, dimethylformamide, or tetrahydrofuran in the presence of an added base such as pyridine, triethylamine, or sodium bicarbonate, at temperatures typically from below ambient to ambient, or up to the boiling point of the solvent or solvent mixture. Couplings of this kind are known to one skilled in the art; see for example Journal of Medicinal Chemistry, 2013, 56(5), 2110-2124 and Journal of Medicinal Chemistry, 2007, 50(15), 3651-3660, and Journal of Enzyme Inhibition and Medicinal Chemistry, 2014, 29(4), 582-589.

Compounds of Formula 1 prepared by the methods described above wherein W is O can be converted to the corresponding thioamides wherein W is S using a variety of standard thiating reagents such as phosphorus pentasulfide or 2,4-bis(4-methoxyphenyl)-1,3-dithia-2,4-diphosphetane-2,4-disulfide (Lawesson's reagent). Reactions of this type are well-known see, for example, Heterocycles 1995, 40, 271-278; J. Med. Chem. 2008, 51, 8124-8134; J. Med. Chem. 1990, 33, 2697-706; Synthesis 1989, (5), 396-3977; J. Chem. Soc., Perkin Trans. 1, 1988, 1663-1668; Tetrahedron 1988 44, 3025-3036; and J. Org. Chem. 1988 53(6), 1323-1326.

It is recognized by one skilled in the art that various functional groups can be converted into others to provide different compounds of Formula 1. For example, Compounds of Formula 1 wherein R⁹ is halomethyl can be used to prepare compounds of Formula 1 wherein R⁹ is hydroxymethyl or cyanomethyl. Compounds of Formula 1, or intermediates for their preparation, may contain aromatic nitro groups, which can be reduced to amino groups, and then converted via reactions well-known in the art (e.g., Sandmeyer reaction) to various halides. By similar known reactions, aromatic amines (anilines) can be converted via diazonium salts to phenols, which can then be alkylated to prepare compounds of Formula 1 with alkoxy substituents. Likewise, aromatic halides such as bromides or iodides prepared via the Sandmeyer reaction can react with alcohols under copper-catalyzed conditions, such as the Ullmann reaction or known modifications thereof, to provide compounds of Formula 1 that contain alkoxy substituents. Additionally, some halogen groups, such as fluorine or chlorine, can be displaced with alcohols under basic conditions to provide compounds of Formula 1 containing the corresponding alkoxy substituents. Compounds of Formula 1 or precursors thereof containing a halide, preferably bromide or iodide, are particularly useful intermediates for transition metal-catalyzed cross-coupling reactions to prepare compounds of Formula 1. These types of reactions are well documented in the literature; see, for example, Tsuji in Transition Metal Reagents and Catalysts: Innovations in Organic Synthesis, John Wiley and Sons, Chichester, 2002; Tsuji in Palladium in Organic Synthesis, Springer, 2005; and Miyaura and Buchwald in Cross Coupling Reactions: A Practical Guide, 2002; and references cited therein

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after introduction of the reagents depicted in the individual schemes, additional routine synthetic steps not described in detail may be needed to complete the synthesis of compounds of Formula 1. One skilled in the art will also recognize that it may be necessary to perform a combination of the steps illustrated in the above schemes in an order other than that implied by the particular sequence presented to prepare the compounds of Formula 1.

One skilled in the art will also recognize that compounds of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Synthesis Examples are, therefore, to be construed as merely illustrative, and not limiting of the disclosure in any way whatsoever. Steps in the following Synthesis Examples illustrate a procedure for each step in an overall synthetic transformation, and the starting material for each step may not have necessarily been prepared by a particular preparative run whose procedure is described in other Examples or Steps. Ambient or room temperature is defined as about 20-25° C. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane; “s” means singlet, “d” means doublet, “t” means triplet, “q” means quartet, “m” means multiplet, “dd” means doublet of doublets, “br s” means broad singlet.

### Example 1

### Preparation of N-[4-chloro-3-[[(2,4-difluorophenyl)amino]carbonyl]phenyl]-β,3-bis(trifluoromethyl)benzenepropanamide (Compound 5)

### Step A: Preparation of ethyl 4,4,4-trifluoro-3-[3-(trifluoromethyl)phenyl]-2-butenoate

To a solution of 2,2,2-trifluoro-1-[3-(trifluoromethyl)phenyl]ethanone (2.11 mL, 12.4 mmol) in tetrahydrofuran (40 mL) at 0° C. was added ethyl 2-(diethoxyphosphoryl)acetate (3.69 mL 18.6 mmol) dropwise followed by sodium hydride (60% in oil, 744 mg, 18.6 mmol). The reaction mixture was allowed to warm to room temperature, and then diluted with saturated aqueous ammonium chloride solution and extracted with ethyl acetate/diethyl ether (1:1, 2×100 mL). The combined extracts were dried over magnesium sulfate, filtered and concentrated under reduced pressure. The resulting material was purified by silica gel column chromatography (eluting with ethyl acetate in hexanes) to provide the title compound (2.6 g).

¹H NMR (CDCl₃) δ 7.70 (d, 1H), 7.53 (m, 2H), 7.48 (d, 1H), 6.68 (s, 1H), 4.05 (q, 2H), 1.06, (t, 3H).

### Step B: Preparation of ethyl β,3-bis(trifluoromethyl)benzenepropanoate

A solution of ethyl 4,4,4-trifluoro-3-[3-(trifluoromethyl)phenyl]-2-butenoate (i.e. the product of Step A) (2.6 g, 8.33 mmol) and palladium (5% on carbon, 0.2 g) in ethanol (20 mL) was stirred under hydrogen for 4 h. Celite® (diatomaceous earth) was added and the reaction mixture was concentrated under reduced pressure. The resulting material was purified by silica gel column chromatography (eluting with ethyl acetate in hexanes) to provide the title compound (2.5 g).

¹H NMR (CDCl₃) δ 7.60 (d, 2H), 7.52 (m, 2H), 4.00-4.10 (m, 3H), 3.10 (m, 1H), 2.90 (m, 1H), 1.14 (t, 3H).

### Step C: Preparation of β,3-bis(trifluoromethyl)benzenepropanoic acid

To a solution of ethyl β,3-bis(trifluoromethyl)benzenepropanoate (i.e. the product of Step B) (2.5 g) in ethanol (30 mL) was added a solution of potassium hydroxide (1.34 g, 23.9 mmol) in water (10 mL). The reaction mixture was stirred overnight and then concentrated under reduced pressure to remove the ethanol. The resulting mixture was diluted with hydrochloric acid (1 N aqueous solution, 25 mL) and water (50 mL) and extracted with ethyl acetate (2×75 mL). The combined organic layers were dried over magnesium sulfate, filtered and concentrated under reduced pressure to provide the title compound (2.3 g).

¹H NMR (CDCl₃) δ 7.65 (d, 1H), 7.55 (s, 1H), 7.50 (m, 2H), 3.95 (m, 1H), 3.10 (dd, 1H), 2.95 (m, 1H).

### Step D: Preparation of N-[4-chloro-3-[[(2,4-difluorophenyl)amino]carbonyl]phenyl]-β,3-bis(trifluoromethyl)benzenepropanamide

To a solution of β,3-bis(trifluoromethyl)benzenepropanoic acid (i.e. the product of Step C) (100 mg, 0.345 mmol) in N,N-dimethylformamide (6 mL) was added O-(7-azabenzotriazol-1-yl)-N,N,N′,N′-tetramethyluronium hexafluorophosphate (HATU) (250 mg, 0.345 mmol) and triethylamine (98 μL, 0.7 mmol). The reaction mixture was stirred for 5 minutes, and then 5-amino-2-chloro-N-(2,4-difluorophenyl)benzamide (97 mg, 0.345 mmol) was added. The reaction mixture was stirred overnight, and then diluted with diethyl ether (100 mL), washed with saturated aqueous sodium bicarbonate solution (2×50 mL) and concentrated under reduced pressure. The resulting material was purified by silica gel column chromatography (eluting with ethyl acetate in hexanes) to provide the title compound, a compound of the present invention, as a solid (137 mg).

¹H NMR (CDCl₃) δ 8.45 (s, 1H), 8.20-8.30 (m, 2H), 7.70 (d, 1H), 7.60 (m, 3H), 7.40-7.50 (m, 2H), 7.30 (d, 1H), 6.93 (t, 2H), 4.10 (m, 1H), 3.10 (dd, 1H).

### Example 2

### Preparation of 3,5-dichloro-N-[4-fluoro-3-[[(4-fluorophenyl)amino]carbonyl]phenyl]-β-(trifluoromethyl)benzenepropanamide (Compound 17)

### Step A: Preparation of ethyl 3,5-dichloro-β-(trifluoromethyl)benzenepropenoate

To a solution of 1-(3,5-dichlorophenyl)-2,2,2-trifluoroethanone (5.0 g, 20.57 mmol) in tetrahydrofuran (40 mL) at 0° C. was added ethyl 2-(diethoxyphosphoryl)acetate (6.12 mL 30.86 mmol) dropwise followed by sodium hydride (60% in oil, 1.24 g, 30.86 mmol). The reaction mixture was allowed to warm to room temperature, and then diluted with saturated aqueous ammonium chloride solution and extracted with ethyl acetate/diethyl ether (1:1, 2×100 mL). The combined extracts were dried over magnesium sulfate, filtered and concentrated under reduced pressure. The resulting material was purified by silica gel column chromatography (eluting with ethyl acetate in hexanes) to provide the title compound (3.5 g).

¹H NMR (CDCl₃) δ 7.43 (s, 1H), 7.18 (s, 2H), 6.60 (s, 1H), 4.10 (q, 2H), 1.13 (t, 3H).

### Step B: Preparation of ethyl 3,5-dichloro-β-(trifluoromethyl)benzenepropanoate

A solution of ethyl 3,5-dichloro-β-(trifluoromethyl)benzenepropenoate (i.e. the product of Step A) (3.5 g) and palladium (5% on carbon, 0.3 g) in ethanol (20 mL) was stirred under hydrogen for 4 h. Celite® (diatomaceous earth) was added and the reaction mixture was concentrated under reduced pressure. The resulting material was purified by silica gel column chromatography (eluting with ethyl acetate in hexanes) to provide the title compound (3.0 g).

¹H NMR (CDCl₃) δ 7.38 (s, 1H), 7.20 (s, 2H), 4.10 (q, 2H), 3.80-3.00 (m, 1H), 3.00 (m, 1H), 2.90-2.80 (m, 1H), 1.18 (t, 3H).

### Step C: Preparation of 3,5-dichloro-β-(trifluoromethyl)benzenepropanoic acid

To a solution of ethyl 3,5-dichloro-β-(trifluoromethyl)benzenepropanoate (i.e. the product of Step B) (3.0 g) in ethanol (30 mL) was added a solution of potassium hydroxide (1.3 g, 23.1 mmol) in water (10 mL). The reaction was stirred overnight and then concentrated under reduced pressure to remove the ethanol. The resulting mixture was diluted with hydrochloric acid (1 N aqueous solution, 25 mL) and water (50 mL) and extracted with ethyl acetate (2×75 mL). The combined organic layers were dried over magnesium sulfate, filtered and concentrated under reduced pressure to provide the title compound (2.3 g).

¹H NMR (CDCl₃) δ 7.35 (s, 1H), 7.20 (s, 2H), 3.90-3.80 (m, 1H), 3.10 (dd, 1H), 2.90 (m, 1H).

### Step D: Preparation of 3,5-dichloro-N-[4-fluoro-3-[[(4-fluorophenyl)amino]carbonyl]-phenyl]-β-(trifluoromethyl)benzenepropanamide

To a solution of 3,5-dichloro-β-(trifluoromethyl)benzenepropanoic acid (i.e. the product of Step C) (50 mg, 0.174 mmol) in N,N-dimethylformamide (6 mL) was added O-(7-azabenzotriazol-1-yl)-N,N,N′,N′-tetramethyluronium hexafluorophosphate (HATU) (127 mg, 0.174 mmol) and triethylamine (50 μL, 0.357 mmol). The reaction mixture was stirred for 5 minutes, and then 5-amino-2-fluoro-N-(4-fluorophenyl)benzamide (43 mg, 0.174 mmol) was added. The reaction mixture was stirred overnight, and then diluted with diethyl ether (100 mL), washed with saturated aqueous sodium bicarbonate solution (2×50 mL) and concentrated under reduced pressure. The resulting material was purified by silica gel column chromatography (eluting with ethyl acetate in hexanes) to provide the title compound, a compound of the present invention, as a solid (41 mg).

¹H NMR (CDCl₃) δ 8.52 (d, 1H), 8.40 (br s, 1H), 8.10 (m, 1H), 7.90 (m, 1H), 7.60 (m, 2H), 7.29 (s, 1H), 7.15-7.05 (m, 5H), 4.00 (m, 1H), 2.90 (m, 1H), 2.70-2.65 (m, 1H).

### Example 3

### Preparation of N-[4-chloro-3-[[(2,4-difluorophenyl)amino]carbonyl]phenyl]-N-methyl-β,3-bis(trifluoromethyl)benzenepropanamide (Compound 145)

### Step A: Preparation of methyl 2-chloro-5-[[4,4,4-trifluoro-3-[3-(trifluoromethyl)phenyl]butanoyl]amino]benzoate

To a stirred solution of β,3-bis(trifluoromethyl)benzenepropanoic acid (1 g, 3.49 mmol, i.e. the product of Example 1 Step C) and methyl 5-amino-2-chlorobenzoate (645 mg, 3.49 mmol) in dimethylformamide (20 ml) was added triethylamine (1.46 ml, 10.48 mmol) at 0° C., followed by propylphosphonic anhydride (T3P, 4.4 ml, 6.98 mmol) and the reaction mixture was warmed to room temperature and stirred for 16 hours. TLC analysis (50% EtOAc/Pet ether) showed completion of the reaction. The reaction was cooled to 0° C., diluted with water (50 ml) and extracted with ethyl acetate. The combined organic layers were dried over sodium sulfate and the solvent was evaporated to give the crude product which was charged on a silica gel column. Elution with 15% ethyl acetate/pet ether gave the title compound (880 mg, 62% yield) as off-white solid.

1H NMR (400 MHz, CDCl3): δ=7.81 (d, 1H), 7.61 (s, 2H), 7.56-7.48 (m, 3H), 7.37 (d, 1H), 7.21 (bs, 1H), 4.22-4.17 (m, 1H), 3.9 (s, 3H), 3.18-3.13 (dd, 1H), 2.91-2.84 (q, 1H); MS (EI): m/z 453 (M+1,100).

### Step B: Preparation of 2-chloro-5-(4,4,4-trifluoro-N-methyl-3-(3-(trifluoromethyl)phenyl)butanamido)benzoic acid¹

To a solution of the compound from Step A (1 g, 2.21 mmol) in tetrahydrofuran (20 ml) was added sodium hydride (163 mg, 4.42 mmol) and methyl iodide (0.55 ml, 8.84 mmol). The reaction mixture was stirred at room temperature for 16 hours. TLC analysis (50% EtOAc/Pet ether) showed completion of the reaction. The reaction was quenched with water (50 ml) and extracted with ethyl acetate. The combined organic extracts were dried over sodium sulfate and the solvent was evaporated to give 1 gram of crude product as the corresponding title acid. LC-MS indicated 80% purity which was used in the next step without further purification.

¹H NMR (400 MHz, DMSO-d₆): δ=13.59 (bs, 1H), 7.77-7.45 (m, 7H), 4.34 (m, 1H), 3.31-2.82 (m, 5H); MS (EI): m/z 453 (M+1,100).

### Step C: Preparation of N-[4-chloro-3-[[(2,4-difluorophenyl)amino]carbonyl]phenyl]-N-methyl-β,3-bis(trifluoromethyl)benzenepropanamide

To a stirred solution of the compound of Step B (1 g, 2.21 mmol) in dimethylformamide (20 ml) was added 2,4-difluoroaniline (284 mg, 2.21 mmol), triethylamine (0.93 ml, 6.63 mmol) and propylphosphonic anhydride (T3P, 1.73 ml, 4.42 mmol). The reaction mixture was stirred at room temperature for 16 hours. TLC analysis (50% EtOAc/pet ether) showed completion of the reaction. The reaction was quenched with water (50 ml) and extracted with ethyl acetate. The combined organic extracts were dried over sodium sulfate and the solvent was evaporated to give the crude product which was charged on a silica gel column. Elution with 20% EtOAc/pet ether gave the title product (650 mg, 52% yield) as a solid.

¹H NMR (400 MHz, DMSO-d₆) VT at 100° C.: δ=10.00 (bs, 1H), 7.78 (d, 1H), 7.68-7.55 (m, 5H), 7.41 (d, 1H), 7.32 (dd, 1H), 7.25 (m, 1H), 7.09 (m, 1H), 4.29 (m, 1H), 3.15 (s, 3H), 2.94 (d, 2H); MS (EI): m/z 564 (M+1,100).

### Example 4

### Preparation of 6-chloro-4-(2,4-dichloro-3,5-dimethoxyphenyl)-5-(2,6-difluorophenyl)-2-ethylpyridazin-3(2H)-one^{#} (Compound 146) and 5-((4S)-1-(λ¹-azaneyl)-5,5,5-trifluoro-2-oxo-4-(3-(trifluoromethyl)phenyl)pentyl)-2-chloro-N-(2,4-difluorophenyl)benzamide^{#} (Compound 147)

The enantiomers of the compound in Example 3 (550 mg) were separated by Supercritical Fluid Chromatography (SFC). The SFC chiral separation was performed using the following conditions to afford 200 mg of Compound 146 and 200 mg of Compound 147.

### Preparative SFC Conditions

### Column/dimensions: (R, R) Whelk-01 (250×30×5μ)

### % CO2: 75% (100% Methanol)

% Co solvent: 25%
Total Flow: 100 g/min

### Back Pressure: 120.0 bar

### Temperature: 30.0° C.

### UV: 214.0 nm

Stack time: 6.7 min
Load/Inj: 108.6 mg/injection

Compound 146: 1H NMR (500 MHz, CDCl3): δ=8.41-8.38 (t, 1H), 8.29 (bs, 1H), 7.61-7.55 (m, 3H), 7.51-7.44 (m, 3H), 7.07 (d, 1H), 6.98 (dd, 2H), 4.22-4.16 (m, 1H), 3.17 (s, 3H), 2.79 (d, 1H), 2.68 (d, 1H); MS (EI): m/z 564 (M+1,100);

Chiral purity: 99.90% ee;

Specific Optical Rotation [α]₂₅ (MeOH, 0.5%)=+55.92

Compound 147: 1H NMR (500 MHz, CDCl3): δ=8.41-8.38 (t, 1H), 8.29 (bs, 1H), 7.61-7.55 (m, 3H), 7.51-7.44 (m, 3H), 7.07 (d, 1H), 6.98 (dd, 2H), 4.24-4.15 (m, 1H), 3.17 (s, 3H), 2.79 (d, 1H), 2.68 (d, 1H); MS (EI): m/z 564 (M+1,100);

Chiral purity: 99.96% ee;

Specific Optical Rotation [α]₂₅ (MeOH, 0.5%)=−55.10

### Example 5

### Preparation of (βR)—N-[4-chloro-3-[[(2,4-difluorophenyl)amino]carbonyl]phenyl]-β,3-bis(trisfluoromethyl)benzenepropanamide (Compound 103) and (S)-2-chloro-N-(2,4-difluorophenyl)-5-(4,4,4-trifluoro-3-(3-(trifluoromethyl)phenyl)butanamido)benzamide^{#}(Compound 104)

The enantiomers of the compound of Example 1 (375 mg) were separated by Supercritical Fluid Chromatography (SFC). The SFC chiral separation was performed using the following conditions to afford 100 mg of Compound 103 and 80 mg of Compound 104.

### Preparative SFC Conditions:

Column/dimensions: (R,R)Whelk-01 (30×250 mm), 5p

### % CO2: 80.0%

% Co solvent: 20.0% (100% Methanol)
Total Flow: 60.0 g/ml

### Back Pressure: 100.0 bar

### Temperature: 30.0° C.

### UV: 254 nm

Stack time: 4 min
Load/Inj: 19.68 mg/injection

Compound 103: 1H NMR (400 MHz, CDCl3): δ=8.38-8.32 (m, 1H), 8.20 (bs, 1H), 7.73 (dd, 2H), 7.62 (d, 2H), 7.56 (d, 1H), 7.51 (t, 1H), 7.42 (t, 2H), 6.95-6.89 (m, 2H), 4.21-4.16 (m, 1H), 3.18 (dd, 1H), 2.91 (q, 1H); MS (EI): m/z 550 (M+1,100);

Chiral purity: 99.90% ee;
Specific Optical Rotation [α]₂₅ (CHCl₃, 0.4%)=−35.31°

Compound 104: 1H NMR (400 MHz, CDCl₃): δ=8.38-8.32 (m, 1H), 8.20 (bs, 1H), 7.73 (d, 1H), 7.70 (dd, 1H), 7.62 (d, 2H), 7.56 (d, 1H), 7.51 (t, 1H), 7.42 (s, 1H), 7.39 (d, 1H), 6.96-6.89 (m, 2H), 4.21-4.14 (m, 1H), 3.18 (dd, 1H), 2.91 (q, 1H); MS (EI): m/z 550 (M+1,100);

Chiral purity: 99.45% ee;
Specific Optical Rotation [α]₂₅ (CHCl₃,0.4%)=+33.32°
^{#}Name was generated by ChemDraw Professional vers. 17.0.

Tables 1-3 disclose specific chiral compounds Formula 2 which are useful as process intermediates for preparing chiral compounds of Formula 1, as described in Scheme 2 above.

TABLE 1

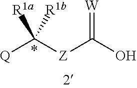

R^{1a} is CF₃, R^{1b} is H, Z is CH₂ and W is O.
Q
3-F—Ph
3-Cl—Ph
3-Br—Ph
3-CF₃—Ph
3,4-di-F—Ph
3-F-4-Cl—Ph
3-F-4-Br—Ph
3-F-4-CF₃—Ph
3,4-di-Cl—Ph
3-Cl-4-Br—Ph
3-Cl-4-CF₃—Ph
3,4-di-Br—Ph
3-Br-4-CF₃—Ph
3-CF3-4-F
3-CF3-4-Cl
3,4-di-(CF₃)—Ph
3,5-di-F—Ph
3-F-5-Cl—Ph
3-F-5-Br—Ph
3-F-5-CF₃—Ph
3,5-di-Cl—Ph
3-Cl-5-Br—Ph
3-Cl-5-CF₃—Ph
3,5-di-Br—Ph
3-Br-5-CF₃—Ph
3,4,5-tri-F—Ph
3,4,5-tri-Cl—Ph
3,5-di-Cl-4-F—Ph
3-CF3-4-Br

The present disclosure also includes Tables 1A through 11A, each of which is constructed the same as Table 1 above, except that the row heading in Table 1 (i.e. “R^{1a} is CF₃, R^{1b} is H, Z is CH₂ and W is O”) is replaced with the respective row headings shown below.

| Table | Row Heading |
|---|---|
| 1A | R^{1a} is CF₃, R^{1b} is OH, Z is CH₂ and W is O. |
| 2A | R^{1a} is CF₃, R^{1b} is Me, Z is CH₂ and W is O. |
| 3A | R^{1a} is CF₃, R^{1b} is OMe, Z is CH₂ and W is O. |
| 4A | R^{1a} is CF₃, R^{1b} is H, Z is CH₂CH₂ and W is O. |
| 5A | R^{1a} is CF₃, R^{1b} is OH, Z is CH₂CH₂ and W is O. |
| 6A | R^{1a} is CF₃, R^{1b} is Me, Z is CH₂CH₂ and W is O. |
| 7A | R^{1a} is CF₃, R^{1b} is OMe, Z is CH₂CH₂ and W is O. |
| 8A | R^{1a} is CF₃, R^{1b} is H, Z is CH₂ and W is S. |
| 9A | R^{1a} is CF₃, R^{1b} is OH, Z is CH₂ and W is S. |
| 10A | R^{1a} is CF₃, R^{1b} is Me, Z is CH₂ and W is S. |
| 11A | R^{1a} is CF₃, R^{1b} is OMe, Z is CH₂ and W is S. |

TABLE 2

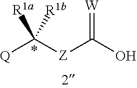

R^{1a} is CF₃, R^{1b} is H, Z is CH₂ and Wis O.
Q
3-F—Ph
3-Cl—Ph
3-Br—Ph
3-CF₃—Ph
3,4-di-F—Ph
3-F-4-Cl—Ph
3-F-4-Br—Ph
3-F-4-CF₃—Ph
3,4-di-Cl—Ph
3-Cl-4-Br—Ph
3-Cl-4-CF₃—Ph
3,4-di-Br—Ph
3-Br-4-CF₃—Ph
3-CF3-4-F
3-CF3-4-Cl
3,4-di-(CF₃)—Ph
3,5-di-F—Ph
3-F-5-Cl—Ph
3-F-5-Br—Ph
3-F-5-CF₃—Ph
3,5-di-Cl—Ph
3-Cl-5-Br—Ph
3-Cl-5-CF₃—Ph
3,5-di-Br—Ph
3-Br-5-CF₃—Ph
3,4,5-tri-F—Ph
3,4,5-tri-Cl—Ph
3,5-di-Cl-4-F—Ph
3-CF3-4-Br

The present disclosure also includes Tables 1B through 11B, each of which is constructed the same as Table 2 above, except that the row heading in Table 2 (i.e. “R^{1a} is CF₃, R^{1b} is H, Z is CH₂ and W is O”) is replaced with the respective row headings shown below.

| Table | Row Heading |
|---|---|
| 1B | R^{1a} is CF₃, R^{1b} is OH, Z is CH₂ and W is O. |
| 2B | R^{1a} is CF₃, R^{1b} is Me, Z is CH₂ and W is O. |
| 3B | R^{1a} is CF₃, R^{1b} is OMe, Z is CH₂ and W is O. |
| 4B | R^{1a} is CF₃, R^{1b} is H, Z is CH₂CH₂ and W is O. |
| 5B | R^{1a} is CF₃, R^{1b} is OH, Z is CH₂CH₂ and W is O. |
| 6B | R^{1a} is CF₃, R^{1b} is Me, Z is CH₂CH₂ and W is O. |
| 7B | R^{1a} is CF₃, R^{1b} is OMe, Z is CH₂CH₂ and W is O. |
| 8B | R^{1a} is CF₃, R^{1b} is H, Z is CH₂ and W is S. |
| 9B | R^{1a} is CF₃, R^{1b} is OH, Z is CH₂ and W is S. |
| 10B | R^{1a} is CF₃, R^{1b} is Me, Z is CH₂ and W is S. |
| 11B | R^{1a} is CF₃, R^{1b} is OMe, Z is CH₂ and W is S. |

TABLE 3

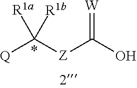

R^{1a} is CF₃, R^{1b} is H, Z is CH₂ and Wis O.
Q
3-F—Ph
3-Cl—Ph
3-Br—Ph
3-CF₃—Ph
3,4-di-F—Ph
3-F-4-Cl—Ph
3-F-4-Br—Ph
3-F-4-CF₃—Ph
3,4-di-Cl—Ph
3-Cl-4-Br—Ph
3-Cl-4-CF₃—Ph
3,4-di-Br—Ph
3-Br-4-CF₃—Ph
3-CF3-4-F
3-CF3-4-Cl
3,4-di-(CF₃)—Ph
3,5-di-F—Ph
3-F-5-Cl—Ph
3-F-5-Br—Ph
3-F-5-CF₃—Ph
3,5-di-Cl—Ph
3-Cl-5-Br—Ph
3-Cl-5-CF₃—Ph
3,5-di-Br—Ph
3-Br-5-CF₃—Ph
3,4,5-tri-F—Ph
3,4,5-tri-Cl—Ph
3,5-di-Cl-4-F—Ph
3-CF3-4-Br

The present disclosure also includes Tables 1C through 11C, each of which is constructed the same as Table 3 above, except that the row heading in Table 3 (i.e. “R^{1a} is CF₃, R^{1b} is H, Z is CH₂ and W is O”) is replaced with the respective row headings shown below.

| Table | Row Heading |
|---|---|
| 1C | R1a is CF3, R1b is OH, Z is CH2 and W is O. |
| 2C | R1a is CF3, R1b is Me, Z is CH2 and W is O. |
| 3C | R1a is CF3, R1b is OMe, Z is CH2 and W is O. |
| 4C | R1a is CF3, R1b is H, Z is CH2CH2 and W is O. |
| 5C | R1a is CF3, R1b is OH, Z is CH2CH2 and W is O. |
| 6C | R1a is CF3, R1b is Me, Z is CH2CH2 and W is O. |
| 7C | R1a is CF3, R1b is OMe, Z is CH2CH2 and W is O. |
| 8C | R1a is CF3, R1b is H, Z is CH2 and W is S. |
| 9C | R1a is CF3, R1b is OH, Z is CH2 and W is S. |
| 10C | R1a is CF3, R1b is Me, Z is CH2 and W is S. |
| 11C | R1a is CF3, R1b is OMe, Z is CH2 and W is S. |

The present invention includes compounds that are enriched compared to the racemic mixture in an enantiomer of Formula 2. When enantiomerically enriched, one enantiomer is present in greater amounts than the other, and the extent of enrichment can be defined by an expression of enantiomeric excess (“ee”), which is defined as (2x−1)·100%, where x is the mole fraction of the dominant enantiomer in the mixture (e.g., an ee of 20% corresponds to a 60:40 ratio of enantiomers). When enriched, the ratio of the (R)-configured or (S)-configured isomers in any compounds of Formula 2, whether produced stereoselectivity or non-stereoselectivity, may take on a broad range of values. For example, compounds of Formula 2 may comprise at least a 50%, or at least a 75%, or at least a 90%, or at least a 95% enantiomeric excess of an isomer. Of particular note are the essentially pure enantiomers of compounds of Formula 2, for example, a compound of Formula 2′ and Formula 2″ (such as those disclosed in Tables 1 or 2).

Embodiments of the present invention also include those described below. In the following Embodiments, reference to “a compound Formula 2” includes the definitions of substituents specified in the Summary of the Invention and in Tables 1 and 2 above.

Embodiment A1. A compound of Formula 2 wherein the (S)-isomer comprises greater than 50% by weight of the total.

Embodiment A2. A compound of Formula 2 wherein the (S)-isomer comprises greater than 60% by weight of the total.

Embodiment A3. A compound of Formula 2 wherein the (S)-isomer comprises greater than 70% by weight of the total.

Embodiment A4. A compound of Formula 2 wherein the (S)-isomer comprises greater than 75% by weight of the total.

Embodiment A5. A compound of Formula 2 wherein the (S)-isomer comprises greater than 80% by weight of the total.

Embodiment A6. A compound of Formula 2 wherein the (S)-isomer comprises greater than 85% by weight of the total.

Embodiment A7. A compound of Formula 2 wherein the (S)-isomer comprises greater than 90% by weight of the total.

Embodiment A8. A compound of Formula 2 wherein the (S)-isomer comprises greater than 95% by weight of the total.

Embodiment A9. A compound of Formula 2 wherein the (S)-isomer comprises greater than 97% by weight of the total.

Embodiment A10. A compound of Formula 2 wherein the (R)-isomer comprises greater than 50% by weight of the total.

Embodiment A11. A compound of Formula 2 wherein the (R)-isomer comprises greater than 60% by weight of the total.

Embodiment A12. A compound of Formula 2 wherein the (R)-isomer comprises greater than 70% by weight of the total.

Embodiment A13. A compound of Formula 2 wherein the (R)-isomer comprises greater than 75% by weight of the total.

Embodiment A14. A compound of Formula 2 wherein the (R)-isomer comprises greater than 80% by weight of the total.

Embodiment A15. A compound of Formula 2 wherein the (R)-isomer comprises greater than 85% by weight of the total.

Embodiment A16. A compound of Formula 2 wherein the (R)-isomer comprises greater than 90% by weight of the total.

Embodiment A17. A compound of Formula 2 wherein the (R)-isomer comprises greater than 95% by weight of the total.

Embodiment A18. A compound of Formula 2 wherein the (R)-isomer comprises greater than 97% by weight of the total.

Embodiment A19. A compound of Formula 2 wherein the (R)-isomer comprises greater

### Formulation/Utility

A compound of this invention will generally be used as an invertebrate pest control active ingredient in a composition, i.e. formulation, with at least one additional component selected from the group consisting of surfactants, solid diluents and liquid diluents, which serves as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions, oil in water emulsions, flowable concentrates and/or suspoemulsions) and the like, which optionally can be thickened into gels. The general types of aqueous liquid compositions are soluble concentrate, suspension concentrate, capsule suspension, concentrated emulsion, microemulsion, oil in water emulsion, flowable concentrate and suspoemulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water, but occasionally another suitable medium like an aromatic or paraffinic hydrocarbon or vegetable oil. Spray volumes can range from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic uptake.

One way of dispensing the compositions disclosed herein over a target area, such as, but not limited to a crop-containing field, is by using drones. Use of drones or unmanned aerial vehicles (UAVs) in agricultural applications, such as for treating fields with chemical products, is rapidly expanding. A container of chemical products is coupled to the UAV and a material dispensing system mounted to the UAV, and the UAV is piloted above the area to be treated while the chemical product is dispensed.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water- | 0.001-90 | 0-99.999 | 0-15 |
| soluble Granules, Tablets and | | | |
| Powders | | | |
| Oil Dispersions, Suspensions, | 1-50 | 40-99 | 0-50 |
| Emulsions, Solutions | | | |
| (including Emulsifiable | | | |
| Concentrates) | | | |
| Dusts | 1-25 | 70-99 | 0-5 |
| Granules and Pellets | 0.001-99 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, N.J.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), alkyl phosphates (e.g., triethylphosphate), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters alkyl and aryl benzoates, γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol, cresol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. Nos. 4,144,050, 3,920,442 and DE 3,246,493. Tablets can be prepared as taught in U.S. Pat. Nos. 5,180,587, 5,232,701 and 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, U K, 2000.

In the following Examples, all formulations are prepared in conventional ways. Compound numbers refer to compounds in Index Tables A-B. Without further elaboration, it is believed that one skilled in the art using the preceding description can utilize the present invention to its fullest extent. The following Examples are, therefore, to be construed as merely illustrative, and not limiting of the disclosure in any way whatsoever. Percentages are by weight except where otherwise indicated.

### Example A

High Strength Concentrate

| | |
|---|---|
| Compound 1 | 98.5% |
| silica aerogel | 0.5% |
| synthetic amorphous fine silica | 1.0% |

### Example B

Wettable Powder

| | |
|---|---|
| Compound 5 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

Granule

| | |
|---|---|
| Compound 8 | 10.0% |
| attapulgite granules (low volatile matter, | 90.0% |
| 0.71/0.30 mm; U.S.S. No. 25-50 sieves) | |

### Example D

Extruded Pellet

| | |
|---|---|
| Compound 32 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

Emulsifiable Concentrate

| | |
|---|---|
| Compound 52 | 10.0% |
| polyoxyethylene sorbitol hexoleate | 20.0% |
| C₆—C₁₀ fatty acid methyl ester | 70.0% |

### Example F

Microemulsion

| | |
|---|---|
| Compound 60 | 5.0% |
| polyvinylpyrrolidone-vinyl acetate copolymer | 30.0% |
| alkylpolyglycoside | 30.0% |
| glyceryl monooleate | 15.0% |
| water | 20.0% |

### Example G

Seed Treatment

| | |
|---|---|
| Compound 76 | 20.00% |
| polyvinylpyrrolidone-vinyl acetate copolymer | 5.00% |
| montan acid wax | 5.00% |
| calcium ligninsulfonate | 1.00% |
| polyoxyethylene/polyoxypropylene block copolymers | 1.00% |
| stearyl alcohol (POE 20) | 2.00% |
| polyorganosilane | 0.20% |
| colorant red dye | 0.05% |
| water | 65.75% |

### Example H

Fertilizer Stick

| | |
|---|---|
| Compound 82 | 2.5% |
| pyrrolidone-styrene copolymer | 4.8% |
| tristyrylphenyl 16-ethoxylate | 2.3% |
| talc | 0.8% |
| corn starch | 5.0% |
| slow-release fertilizer | 36.0% |
| kaolin | 38.0% |
| water | 10.6% |

### Example I

Suspension Concentrate

| | |
|---|---|
| compound 83 | 35% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| l,2-benzisothiazolin-3-one | 0.1% |
| water | 53.7% |

### Example J

Emulsion in Water

| | |
|---|---|
| compound 85 | 10.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| l,2-benzisothiazolin-3-one | 0.1% |
| aromatic petroleum based hydrocarbon | 20.0 |
| water | 58.7% |

### Example K

Oil Dispersion

| | |
|---|---|
| compound 30 | 25% |
| polyoxyethylene sorbitol hexaoleate | 15% |
| organically modified bentonite clay | 2.5% |
| fatty acid methyl ester | 57.5% |

### Example L

Suspoemulsion

| | |
|---|---|
| compound 1 | 10.0% |
| imidacloprid | 5.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| l,2-benzisothiazolin-3-one | 0.1% |
| aromatic petroleum based hydrocarbon | 20.0% |
| water | 53.7% |

Compounds of this invention exhibit activity against a wide spectrum of invertebrate pests. These pests include invertebrates inhabiting a variety of environments such as, for example, plant foliage, roots, soil, harvested crops or other foodstuffs, building structures or animal integuments. These pests include, for example, invertebrates feeding on foliage (including leaves, stems, flowers and fruits), seeds, wood, textile fibers or animal blood or tissues, and thereby causing injury or damage to, for example, growing or stored agronomic crops, forests, greenhouse crops, ornamentals, nursery crops, stored foodstuffs or fiber products, or houses or other structures or their contents, or being harmful to animal health or public health. Those skilled in the art will appreciate that not all compounds are equally effective against all growth stages of all pests.

These present compounds and compositions are thus useful agronomically for protecting field crops from phytophagous invertebrate pests, and also nonagronomically for protecting other horticultural crops and plants from phytophagous invertebrate pests. This utility includes protecting crops and other plants (i.e. both agronomic and nonagronomic) that contain genetic material introduced by genetic engineering (i.e. transgenic) or modified by mutagenesis to provide advantageous traits. Examples of such traits include tolerance to herbicides, resistance to phytophagous pests (e.g., insects, mites, aphids, spiders, nematodes, snails, plant-pathogenic fungi, bacteria and viruses), improved plant growth, increased tolerance of adverse growing conditions such as high or low temperatures, low or high soil moisture, and high salinity, increased flowering or fruiting, greater harvest yields, more rapid maturation, higher quality and/or nutritional value of the harvested product, or improved storage or process properties of the harvested products. Transgenic plants can be modified to express multiple traits. Examples of plants containing traits provided by genetic engineering or mutagenesis include varieties of corn, cotton, soybean and potato expressing an insecticidal Bacillus thuringiensis toxin such as YIELD GARD®, KNOCKOUT®, STARLINK®, BOLLGARD®, NuCOTN® and NEWLEAF®, INVICTA RR2 PRO™, and herbicide-tolerant varieties of corn, cotton, soybean and rapeseed such as ROUNDUP READY®, LIBERTY LINK®, IMI®, STS® and CLEARFIELD®, as well as crops expressing N-acetyltransferase (GAT) to provide resistance to glyphosate herbicide, or crops containing the HRA gene providing resistance to herbicides inhibiting acetolactate synthase (ALS). The present compounds and compositions may interact in a greater-than-additive (i.e. enhanced) effect with traits introduced by genetic engineering or modified by mutagenesis, thus enhancing phenotypic expression or effectiveness of the traits or increasing the invertebrate pest control effectiveness of the present compounds and compositions. In particular, the present compounds and compositions may interact in a greater-than-additive (i.e. enhanced) effect with the phenotypic expression of proteins or other natural products toxic to invertebrate pests to provide greater-than-additive control of these pests.

Compositions of this invention can also optionally comprise plant nutrients, e.g., a fertilizer composition comprising at least one plant nutrient selected from nitrogen, phosphorus, potassium, sulfur, calcium, magnesium, iron, copper, boron, manganese, zinc, and molybdenum. Of note are compositions comprising at least one fertilizer composition comprising at least one plant nutrient selected from nitrogen, phosphorus, potassium, sulfur, calcium and magnesium. Compositions of the present invention which further comprise at least one plant nutrient can be in the form of liquids or solids. Of note are solid formulations in the form of granules, small sticks or tablets. Solid formulations comprising a fertilizer composition can be prepared by mixing the compound or composition of the present invention with the fertilizer composition together with formulating ingredients and then preparing the formulation by methods such as granulation or extrusion. Alternatively solid formulations can be prepared by spraying a solution or suspension of a compound or composition of the present invention in a volatile solvent onto a previous prepared fertilizer composition in the form of dimensionally stable mixtures, e.g., granules, small sticks or tablets, and then evaporating the solvent.

Nonagronomic uses refer to invertebrate pest control in the areas other than fields of crop plants. Nonagronomic uses of the present compounds and compositions include control of invertebrate pests in stored grains, beans and other foodstuffs, and in textiles such as clothing and carpets. Nonagronomic uses of the present compounds and compositions also include invertebrate pest control in ornamental plants, forests, in yards, along roadsides and railroad rights of way, and on turf such as lawns, golf courses and pastures. Nonagronomic uses of the present compounds and compositions also include invertebrate pest control in houses and other buildings which may be occupied by humans and/or companion, farm, ranch, zoo or other animals. Nonagronomic uses of the present compounds and compositions also include the control of pests such as termites that can damage wood or other structural materials used in buildings.

Nonagronomic uses of the present compounds and compositions also include protecting human and animal health by controlling invertebrate pests that are parasitic or transmit infectious diseases. The controlling of animal parasites includes controlling external parasites that are parasitic to the surface of the body of the host animal (e.g., shoulders, armpits, abdomen, inner part of the thighs) and internal parasites that are parasitic to the inside of the body of the host animal (e.g., stomach, intestine, lung, veins, under the skin, lymphatic tissue). External parasitic or disease transmitting pests include, for example, chiggers, ticks, lice, mosquitoes, flies, mites and fleas. Internal parasites include heartworms, hookworms and helminths.

Compounds and compositions of the present invention are suitable for systemic and/or non-systemic control of infestation or infection by parasites on animals. Compounds and compositions of the present invention are particularly suitable for combating external parasitic or disease transmitting pests. Compounds and compositions of the present invention are suitable for combating parasites that infest agricultural working animals, such as cattle, sheep, goats, horses, pigs, donkeys, camels, buffalos, rabbits, hens, turkeys, ducks, geese and bees; pet animals and domestic animals such as dogs, cats, pet birds and aquarium fish; as well as so-called experimental animals, such as hamsters, guinea pigs, rats and mice. By combating these parasites, fatalities and performance reduction (in terms of meat, milk, wool, skins, eggs, honey, etc.) are reduced, so that applying a composition comprising a compound of the present invention allows more economic and simple husbandry of animals.

Examples of agronomic or nonagronomic invertebrate pests include eggs, larvae and adults of the order Lepidoptera, such as armyworms, cutworms, loopers, and heliothines in the family Noctuidae (e.g., pink stem borer (Sesamia inferens Walker), corn stalk borer (Sesamia nonagrioides Lefebvre), southern armyworm (Spodoptera eridania Cramer), fall armyworm (Spodoptera frugiperda J. E. Smith), beet armyworm (Spodoptera exigua Hubner), cotton leafworm (Spodoptera linoralis Boisduval), yellowstriped armyworm (Spodoptera ornithogalli Guende), black cutworm (Agrotis ipsilon Hufnagel), velvetbean caterpillar (Anticarsia gemmatalis Hubner), green fruitworm (Lithophane antennata Walker), cabbage armyworm (Barathra brassicae Linnaeus), soybean looper (Pseudoplusia includens Walker), cabbage looper (Trichoplusia ni Hubner), tobacco budworm (Heliothis virescens Fabricius)); borers, casebearers, webworms, coneworms, cabbageworms and skeletonizers from the family Pyralidae (e.g., European corn borer (Ostrinia nubilalis Hubner), navel orangeworm (Amyelois transitella Walker), corn root webworm (Crambus caliginosellus Clemens), sod webworms (Pyralidae: Crambinae) such as sod worm (Herpetogramma licarsisalis Walker), sugarcane stem borer (Chilo infuscatellus Snellen), tomato small borer (Neoleucinodes elegantalis Guende), green leafroller (Cnaphalocrocis medinalis), grape leaffolder (Desmia funeralis Hubner), melon worm (Diaphania nitidalis Stoll), cabbage center grub (Helluala hydralis Guende), yellow stem borer (Scirpophaga incertulas Walker), early shoot borer (Scirpophaga infuscatellus Snellen), white stem borer (Scirpophaga innotata Walker), top shoot borer (Scirpophaga nivella Fabricius), dark-headed rice borer (Chilo polychrysus Meyrick), striped riceborer (Chilo suppressalis Walker), cabbage cluster caterpillar (Crocidolomia binotalis English)); leafrollers, budworms, seed worms, and fruit worms in the family Tortricidae (e.g., codling moth (Cydia pomonella Linnaeus), grape berry moth (Endopiza viteana Clemens), oriental fruit moth (Grapholita molesta Busck), citrus false codling moth (Cryptophlebia leucotreta Meyrick), citrus borer (Ecdytolopha aurantiana Lima), redbanded leafroller (Argyrotaenia velutinana Walker), obliquebanded leafroller (Choristoneura rosaceana Harris), light brown apple moth (Epiphyas postvittana Walker), European grape berry moth (Eupoecilia ambiguella Hubner), apple bud moth (Pandemis pyrusana Kearfott), omnivorous leafroller (Platynota stultana Walsingham), barred fruit-tree tortrix (Pandemis cerasana Hubner), apple brown tortrix (Pandemis heparana Denis & Schiffermuller)); and many other economically important lepidoptera (e.g., diamondback moth (Plutella xylostella Linnaeus), pink bollworm (Pectinophora gossypiella Saunders), gypsy moth (Lymantria dispar Linnaeus), peach fruit borer (Carposina niponensis Walsingham), peach twig borer (Anarsia lineatella Zeller), potato tuberworm (Phthorimaea operculella Zeller), spotted teniform leafminer (Lithocolleis blancardella Fabricius), Asiatic apple leafminer (Lithocolletis ringoniella Matsumura), rice leaffolder (Lerodea eufala Edwards), apple leafminer (Leucoptera scitella Zeller)); eggs, nymphs and adults of the order Blattodea including cockroaches from the families Blattellidae and Blattidae (e.g., oriental cockroach (Blatta orientalis Linnaeus), Asian cockroach (Blatella asahinai Mizukubo), German cockroach (Blattella germanica Linnaeus), brownbanded cockroach (Supella longipalpa Fabricius), American cockroach (Periplaneta americana Linnaeus), brown cockroach (Periplaneta brunnea Burmeister), Madeira cockroach (Leucophaea maderae Fabricius)), smoky brown cockroach (Periplaneta fuliginosa Service), Australian Cockroach (Periplaneta australasiae Fabr.), lobster cockroach (Nauphoeta cinerea Olivier) and smooth cockroach (Symploce pallens Stephens)); eggs, foliar feeding, fruit feeding, root feeding, seed feeding and vesicular tissue feeding larvae and adults of the order Coleoptera including weevils from the families Anthribidae, Bruchidae, and Curculionidae (e.g., boll weevil (Anthonomus grandis Boheman), rice water weevil (Lissorhoptrus oryzophilus Kuschel), granary weevil (Sitophilus granarius Linnaeus), rice weevil (Sitophilus oryzae Linnaeus)), annual bluegrass weevil (Listronotus maculicollis Dietz), bluegrass billbug (Sphenophorus parvulus Gyllenhal), hunting billbug (Sphenophorus venatus vestitus), Denver billbug (Sphenophorus cicatristriatus Fahraeus)); flea beetles, cucumber beetles, rootworms, leaf beetles, potato beetles, and leafminers in the family Chrysomelidae (e.g., Colorado potato beetle (Leptinotarsa decemlineata Say), western corn rootworm (Diabrotica virgifera virgifera LeConte)); chafers and other beetles from the family Scarabaeidae (e.g., Japanese beetle (Popillia japonica Newman), oriental beetle (Anomala orientalis Waterhouse, Exomala orientalis (Waterhouse) Baraud), northern masked chafer (Cyclocephala borealis Arrow), southern masked chafer (Cyclocephala immaculata Olivier or C. lurida Bland), dung beetle and white grub (Aphodius spp.), black turfgrass ataenius (Ataenius spretulus Haldeman), green June beetle (Cotinis nitida Linnaeus), Asiatic garden beetle (Maladera castanea Arrow), May/June beetles (Phyllophaga spp.) and European chafer (Rhizotrogus majalis Razoumowsky)); carpet beetles from the family Dermestidae; wireworms from the family Elateridae; bark beetles from the family Scolytidae and flour beetles from the family Tenebrionidae.

In addition, agronomic and nonagronomic pests include: eggs, adults and larvae of the order Dermaptera including earwigs from the family Forficulidae (e.g., European earwig (Forficula auricularia Linnaeus), black earwig (Chelisoches morio Fabricius)); eggs, immatures, adults and nymphs of the orders Hemiptera and Homoptera such as, plant bugs from the family Miridae, cicadas from the family Cicadidae, leafhoppers (e.g. Empoasca spp.) from the family Cicadellidae, bed bugs (e.g., Cimex lectularius Linnaeus) from the family Cimicidae, planthoppers from the families Fulgoroidae and Delphacidae, treehoppers from the family Membracidae, psyllids from the family Psyllidae, whiteflies from the family Aleyrodidae, aphids from the family Aphididae, phylloxera from the family Phylloxeridae, mealybugs from the family Pseudococcidae, scales from the families Coccidae, Diaspididae and Margarodidae, lace bugs from the family Tingidae, stink bugs from the family Pentatomidae, chinch bugs (e.g., hairy chinch bug (Blissus leucopterus hirtus Montandon) and southern chinch bug (Blissus insularis Barber)) and other seed bugs from the family Lygaeidae, spittlebugs from the family Cercopidae squash bugs from the family Coreidae, and red bugs and cotton stainers from the family Pyrrhocoridae.

Agronomic and nonagronomic pests also include: eggs, larvae, nymphs and adults of the order Acari (mites) such as spider mites and red mites in the family Tetranychidae (e.g., European red mite (Panonychus ulmi Koch), two spotted spider mite (Tetranychus urticae Koch), McDaniel mite (Tetranychus mcdanieli McGregor)); flat mites in the family Tenuipalpidae (e.g., citrus flat mite (Brevipalpus lewisi McGregor)); rust and bud mites in the family Eriophyidae and other foliar feeding mites and mites important in human and animal health, i.e. dust mites in the family Epidermoptidae, follicle mites in the family Demodicidae, grain mites in the family Glycyphagidae; ticks in the family Ixodidae, commonly known as hard ticks (e.g., deer tick (Ixodes scapularis Say), Australian paralysis tick (Ixodes holocyclus Neumann), American dog tick (Dermacentor variabilis Say), lone star tick (Amblyomma americanum Linnaeus)) and ticks in the family Argasidae, commonly known as soft ticks (e.g., relapsing fever tick (Ornithodoros turicata), common fowl tick (Argas radiatus)); scab and itch mites in the families Psoroptidae, Pyemotidae, and Sarcoptidae; eggs, adults and immatures of the order Orthoptera including grasshoppers, locusts and crickets (e.g., migratory grasshoppers (e.g., Melanoplus sanguinipes Fabricius, M. diferentialis Thomas), American grasshoppers (e.g., Schistocerca americana Drury), desert locust (Schistocerca gregaria Forskal), migratory locust (Locusta migratoria Linnaeus), bush locust (Zonocerus spp.), house cricket (Acheta domesticus Linnaeus), mole crickets (e.g., tawny mole cricket (Scapteriscus vicinus Scudder) and southern mole cricket (Scapteriscus borellii Giglio-Tos)); eggs, adults and immatures of the order Diptera including leafminers (e.g., Liriomyza spp. such as serpentine vegetable leafminer (Liriomyza sativae Blanchard)), midges, fruit flies (Tephritidae), frit flies (e.g., Oscinella frit Linnaeus), soil maggots, house flies (e.g., Musca domestica Linnaeus), lesser house flies (e.g., Fannia canicularis Linnaeus, F. femoralis Stein), stable flies (e.g., Stomoxys calcitrans Linnaeus), face flies, horn flies, blow flies (e.g., Chrysomya spp., Phormia spp.), and other muscoid fly pests, horse flies (e.g., Tabanus spp.), bot flies (e.g., Gastrophilus spp., Oestrus spp.), cattle grubs (e.g., Hypoderma spp.), deer flies (e.g., Chrysops spp.), keds (e.g., Melophagus ovinus Linnaeus) and other Brachycera, mosquitoes (e.g., Aedes spp., Anopheles spp., Culex spp.), black flies (e.g., Prosimulium spp., Simulium spp.), biting midges, sand flies, sciarids, and other Nematocera; eggs, adults and immatures of the order Thysanoptera including onion thrips (Thrips tabaci Lindeman), flower thrips (Frankliniella spp.), and other foliar feeding thrips; insect pests of the order Hymenoptera including ants of the Family Formicidae including the Florida carpenter ant (Camponotus floridanus Buckley), red carpenter ant (Camponotus ferrugineus Fabricius), black carpenter ant (Camponotus pennsylvanicus De Geer), white-footed ant (Technomyrmex albipes fr. Smith), big headed ants (Pheidole sp.), ghost ant (Tapinoma melanocephalum Fabricius); Pharaoh ant (Monomorium pharaonis Linnaeus), little fire ant (Wasmannia auropunctata Roger), fire ant (Solenopsis geminata Fabricius), red imported fire ant (Solenopsis invicta Buren), Argentine ant (Iridomyrmex humilis Mayr), crazy ant (Paratrechina longicornis Latreille), pavement ant (Tetramorium caespitum Linnaeus), cornfield ant (Lasius alienus Forster) and odorous house ant (Tapinoma sessile Say). Other Hymenoptera including bees (including carpenter bees), hornets, yellow jackets, wasps, and sawflies (Neodiprion spp.; Cephus spp.); insect pests of the order Isoptera including termites in the Termitidae (e.g., Macrotermes sp., Odontotermes obesus Rambur), Kalotermitidae (e.g., Cryptotermes sp.), and Rhinotermitidae (e.g., Reticulitermes sp., Coptotermes sp., Heterotermes tenuis Hagen) families, the eastern subterranean termite (Reticulitermes flavipes Kollar), western subterranean termite (Reticulitermes hesperus Banks), Formosan subterranean termite (Coptotermes formosanus Shiraki), West Indian drywood termite (Incisitermes immigrans Snyder), powder post termite (Cryptotermes brevis Walker), drywood termite (Incisitermes snyderi Light), southeastern subterranean termite (Reticulitermes virginicus Banks), western drywood termite (Incisitermes minor Hagen), arboreal termites such as Nasutitermes sp. and other termites of economic importance; insect pests of the order Thysanura such as silverfish (Lepisma saccharina Linnaeus) and firebrat (Thermobia domestica Packard); insect pests of the order Mallophaga and including the head louse (Pediculus humanus capitis De Geer), body louse (Pediculus humanus Linnaeus), chicken body louse (Menacanthus stramineus Nitszch), dog biting louse (Trichodectes canis De Geer), fluff louse (Goniocotes gallinae De Geer), sheep body louse (Bovicola ovis Schrank), short-nosed cattle louse (Haematopinus eurysternus Nitzsch), long-nosed cattle louse (Linognathus vituli Linnaeus) and other sucking and chewing parasitic lice that attack man and animals; insect pests of the order Siphonoptera including the oriental rat flea (Xenopsylla cheopis Rothschild), cat flea (Ctenocephalides felis Bouche), dog flea (Ctenocephalides canis Curtis), hen flea (Ceratophyllus gallinae Schrank), sticktight flea (Echidnophaga gallinacea Westwood), human flea (Pulex irritans Linnaeus) and other fleas afflicting mammals and birds. Additional arthropod pests covered include: spiders in the order Araneae such as the brown recluse spider (Loxosceles reclusa Gertsch & Mulaik) and the black widow spider (Latrodectus mactans Fabricius), and centipedes in the order Scutigeromorpha such as the house centipede (Scutigera coleoptrata Linnaeus).

Examples of invertebrate pests of stored grain include larger grain borer (Prostephanus truncatus), lesser grain borer (Rhyzopertha dominica), rice weevil (Stiophilus oryzae), maize weevil (Stiophilus zeamais), cowpea weevil (Callosobruchus maculatus), red flour beetle (Tribolium castaneum), granary weevil (Stiophilus granarius), Indian meal moth (Plodia interpunctella), Mediterranean flour beetle (Ephestia kuhniella) and flat or rusty grain beetle (Cryptolestis ferrugineus).

Compounds of the present invention may have activity on members of the Classes Nematoda, Cestoda, Trematoda, and Acanthocephala including economically important members of the orders Strongylida, Ascaridida, Oxyurida, Rhabditida, Spirurida, and Enoplida such as but not limited to economically important agricultural pests (i.e. root knot nematodes in the genus Meloidogyne, lesion nematodes in the genus Pratylenchus, stubby root nematodes in the genus Trichodorus, etc.) and animal and human health pests (i.e. all economically important flukes, tapeworms, and roundworms, such as Strongylus vulgaris in horses, Toxocara canis in dogs, Haemonchus contortus in sheep, Dirofilaria immitis Leidy in dogs, Anoplocephala perfoliata in horses, Fasciola hepatica Linnaeus in ruminants, etc.).

Compounds of the invention may have activity against pests in the order Lepidoptera (e.g., Alabama argillacea Hubner (cotton leaf worm), Archips argyrospila Walker (fruit tree leaf roller), A. rosana Linnaeus (European leaf roller) and other Archips species, Chilo suppressalis Walker (rice stem borer), Cnaphalocrosis medinalis Guende (rice leaf roller), Crambus caliginosellus Clemens (corn root webworm), Crambus teterrellus Zincken (bluegrass webworm), Cydia pomonella Linnaeus (codling moth), Earias insulana Boisduval (spiny bollworm), Earias vittella Fabricius (spotted bollworm), Helicoverpa armigera Hubner (American bollworm), Helicoverpa zea Boddie (corn earworm), Heliothis virescens Fabricius (tobacco budworm), Herpetogramma licarsisalis Walker (sod webworm), Lobesia botrana Denis & Schiffermuller (grape berry moth), Pectinophora gossypiella Saunders (pink bollworm), Phyllocnistis citrella Stainton (citrus leafminer), Pieris brassicae Linnaeus (large white butterfly), Pieris rapae Linnaeus (small white butterfly), Plutella xylostella Linnaeus (diamondback moth), Spodoptera exigua Hubner (beet armyworm), Spodoptera litura Fabricius (tobacco cutworm, cluster caterpillar), Spodoptera frugiperda J. E. Smith (fall armyworm), Trichoplusia ni Hubner (cabbage looper) and Tuta absoluta Meyrick (tomato leafminer)).

Compounds of the invention have significant activity on members from the order Homoptera including: Acyrthosiphon pisum Harris (pea aphid), Aphis craccivora Koch (cowpea aphid), Aphis fabae Scopoli (black bean aphid), Aphis gossypii Glover (cotton aphid, melon aphid), Aphis pomi De Geer (apple aphid), Aphis spiraecola Patch (spirea aphid), Aulacorthum solani Kaltenbach (foxglove aphid), Chaetosiphon fragaefolii Cockerell (strawberry aphid), Diuraphis noxia Kurdjumov/Mordvilko (Russian wheat aphid), Dysaphis plantaginea Paaserini (rosy apple aphid), Eriosoma lanigerum Hausmann (woolly apple aphid), Hyalopterus pruni Geoffroy (mealy plum aphid), Lipaphis erysimi Kaltenbach (turnip aphid), Metopolophium dirrhodum Walker (cereal aphid), Macrosiphum euphorbiae Thomas (potato aphid), Myzus persicae Sulzer (peach-potato aphid, green peach aphid), Nasonovia ribisnigri Mosley (lettuce aphid), Pemphigus spp. (root aphids and gall aphids), Rhopalosiphum maidis Fitch (corn leaf aphid), Rhopalosiphum padi Linnaeus (bird cherry-oat aphid), Schizaphis graminum Rondani (greenbug), Sitobion avenae Fabricius (English grain aphid), Therioaphis maculata Buckton (spotted alfalfa aphid), Toxoptera aurantii Boyer de Fonscolombe (black citrus aphid), and Toxoptera citricida Kirkaldy (brown citrus aphid); Adelges spp. (adelgids); Phylloxera devastatrix Pergande (pecan phylloxera); Bemisia tabaci Gennadius (tobacco whitefly, sweetpotato whitefly), Bemisia argentifolii Bellows & Perring (silverleaf whitefly), Dialeurodes citri Ashmead (citrus whitefly) and Trialeurodes vaporariorum Westwood (greenhouse whitefly); Empoasca fabae Harris (potato leafhopper), Laodelphax striatellus Fallen (smaller brown planthopper), Macrolestes quadrilineatus Forbes (aster leafhopper), Nephotettix cinticeps Uhler (green leafhopper), Nephotettix nigropictus Stål (rice leafhopper), Nilaparvata lugens Stål (brown planthopper), Peregrinus maidis Ashmead (corn planthopper), Sogatella furcifera Horvath (white-backed planthopper), Sogatodes orizicola Muir (rice delphacid), Typhlocyba pomaria McAtee white apple leafhopper, Erythroneoura spp. (grape leafhoppers); Magicidada septendecim Linnaeus (periodical cicada); Icerya purchasi Maskell (cottony cushion scale), Quadraspidiotus perniciosus Comstock (San Jose scale); Planococcus citri Risso (citrus mealybug); Pseudococcus spp. (other mealybug complex); Cacopsylla pyricola Foerster (pear psylla), Trioza diospyri Ashmead (persimmon psylla).

Compounds of this invention also have activity on members from the order Hemiptera including: Acrosternum hilare Say (green stink bug), Anasa tristis De Geer (squash bug), Blissus leucopterus leucopterus Say (chinch bug), Cimex lectularius Linnaeus (bed bug) Corythuca gossypii Fabricius (cotton lace bug), Cyrtopeltis modesta Distant (tomato bug), Dysdercus suturellus Herrich-Schaffer (cotton stainer), Euchistus servus Say (brown stink bug), Euchistus variolarius Palisot de Beauvois (one-spotted stink bug), Graptosthetus spp. (complex of seed bugs), Halymorpha halys Stål (brown marmorated stink bug), Leptoglossus corculus Say (leaf-footed pine seed bug), Lygus lineolaris Palisot de Beauvois (tarnished plant bug), Nezara viridula Linnaeus (southern green stink bug), Oebalus pugnax Fabricius (rice stink bug), Oncopeltus fasciatus Dallas (large milkweed bug), Pseudatomoscelis seriatus Reuter (cotton fleahopper). Other insect orders controlled by compounds of the invention include Thysanoptera (e.g., Frankliniella occidentalis Pergande (western flower thrips), Scirthothrips citri Moulton (citrus thrips), Sericothrips variabilis Beach (soybean thrips), and Thrips tabaci Lindeman (onion thrips); and the order Coleoptera (e.g., Leptinotarsa decemlineata Say (Colorado potato beetle), Epilachna varivestis Mulsant (Mexican bean beetle) and wireworms of the genera Agriotes, Athous or Limonius).

Note that some contemporary classification systems place Homoptera as a suborder within the order Hemiptera.

Of note is use of compounds of this invention for controlling western flower thrips (Frankliniella occidentalis). Of note is use of compounds of this invention for controlling potato leafhopper (Empoasca fabae). Of note is use of compounds of this invention for controlling cotton melon aphid (Aphis gossypii). Of note is use of compounds of this invention for controlling green peach aphid (Myzus persicae). Of note is use of compounds of this invention for controlling sweetpotato whitefly (Bemisia tabaci).

Compounds of the present invention may also be useful for increasing vigor of a crop plant. This method comprises contacting the crop plant (e.g., foliage, flowers, fruit or roots) or the seed from which the crop plant is grown with a compound of Formula 1 in amount sufficient to achieve the desired plant vigor effect (i.e. biologically effective amount). Typically the compound of Formula 1 is applied in a formulated composition. Although the compound of Formula 1 is often applied directly to the crop plant or its seed, it can also be applied to the locus of the crop plant, i.e. the environment of the crop plant, particularly the portion of the environment in close enough proximity to allow the compound of Formula 1 to migrate to the crop plant. The locus relevant to this method most commonly comprises the growth medium (i.e. medium providing nutrients to the plant), typically soil in which the plant is grown. Treatment of a crop plant to increase vigor of the crop plant thus comprises contacting the crop plant, the seed from which the crop plant is grown or the locus of the crop plant with a biologically effective amount of a compound of Formula 1.

Increased crop vigor can result in one or more of the following observed effects: (a) optimal crop establishment as demonstrated by excellent seed germination, crop emergence and crop stand; (b) enhanced crop growth as demonstrated by rapid and robust leaf growth (e.g., measured by leaf area index), plant height, number of tillers (e.g., for rice), root mass and overall dry weight of vegetative mass of the crop; (c) improved crop yields, as demonstrated by time to flowering, duration of flowering, number of flowers, total biomass accumulation (i.e. yield quantity) and/or fruit or grain grade marketability of produce (i.e. yield quality); (d) enhanced ability of the crop to withstand or prevent plant disease infections and arthropod, nematode or mollusk pest infestations; and (e) increased ability of the crop to withstand environmental stresses such as exposure to thermal extremes, suboptimal moisture or phytotoxic chemicals.

The compounds of the present invention may increase the vigor of treated plants compared to untreated plants by killing or otherwise preventing feeding of phytophagous invertebrate pests in the environment of the plants. In the absence of such control of phytophagous invertebrate pests, the pests reduce plant vigor by consuming plant tissues or sap, or transmitting plant pathogens such as viruses. Even in the absence of phytophagous invertebrate pests, the compounds of the invention may increase plant vigor by modifying metabolism of plants. Generally, the vigor of a crop plant will be most significantly increased by treating the plant with a compound of the invention if the plant is grown in a nonideal environment, i.e. an environment comprising one or more aspects adverse to the plant achieving the full genetic potential it would exhibit in an ideal environment.

Of note is a method for increasing vigor of a crop plant wherein the crop plant is grown in an environment comprising phytophagous invertebrate pests. Also of note is a method for increasing vigor of a crop plant wherein the crop plant is grown in an environment not comprising phytophagous invertebrate pests. Also of note is a method for increasing vigor of a crop plant wherein the crop plant is grown in an environment comprising an amount of moisture less than ideal for supporting growth of the crop plant. Of note is a method for increasing vigor of a crop plant wherein the crop is rice. Also of note is a method for increasing vigor of a crop plant wherein the crop is maize (corn). Also of note is a method for increasing vigor of a crop plant wherein the crop is soybean.

Compounds of this invention can also be mixed with one or more other biologically active compounds or agents including insecticides, fungicides, nematocides, bactericides, acaricides, herbicides, herbicide safeners, growth regulators such as insect molting inhibitors and rooting stimulants, chemosterilants, semiochemicals, repellents, attractants, pheromones, feeding stimulants, other biologically active compounds or entomopathogenic bacteria, virus or fungi to form a multi-component pesticide giving an even broader spectrum of agronomic and nonagronomic utility. Thus, the present invention also pertains to a composition comprising a biologically effective amount of a compound of Formula 1, at least one additional component selected from the group consisting of surfactants, solid diluents and liquid diluents, and at least one additional biologically active compound or agent. For mixtures of the present invention, the other biologically active compounds or agents can be formulated together with the present compounds, including the compounds of Formula 1, to form a premix, or the other biologically active compounds or agents can be formulated separately from the present compounds, including the compounds of Formula 1, and the two formulations combined together before application (e.g., in a spray tank) or, alternatively, applied in succession.

Examples of such biologically active compounds or agents with which compounds of this invention can be formulated are insecticides such as abamectin, acephate, acequinocyl, acetamiprid, acrinathrin, afidopyropen ([(3S,4R,4aR,6S,6aS,12R,12aS,12bS)-3-[(cyclopropylcarbonyl)oxy]-1,3,4,4a,5,6,6a,12,12a,12b-decahydro-6,12-dihydroxy-4,6a,12b-trimethyl-11-oxo-9-(3-pyridinyl)-2H,11H-naphtho[2,1-b]pyrano[3,4-e]pyran-4-yl]methyl cyclopropanecarboxylate), amidoflumet, amitraz, avermectin, azadirachtin, azinphos-methyl, benfuracarb, bensultap, bifenthrin, bifenazate, bistrifluron, borate, buprofezin, cadusafos, carbaryl, carbofuran, cartap, carzol, chlorantraniliprole, chlorfenapyr, chlorfluazuron, chlorpyrifos, chlorpyrifos-methyl, chromafenozide, clofentezin, clothianidin, cyantraniliprole (3-bromo-1-(3-chloro-2-pyridinyl)-N-[4-cyano-2-methyl-6-[(methylamino)carbonyl]phenyl]-1H-pyrazole-5-carboxamide), cyclaniliprole (3-bromo-N-[2-bromo-4-chloro-6-[[(1-cyclopropylethyl)amino]carbonyl]phenyl]-1-(3-chloro-2-pyridinyl)-1H-pyrazole-5-carboxamide), cycloprothrin, cycloxaprid ((5S,8R)-1-[(6-chloro-3-pyridinyl)methyl]-2,3,5,6,7,8-hexahydro-9-nitro-5,8-Epoxy-1H-imidazo[1,2-a]azepine) cyflumetofen, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, diafenthiuron, diazinon, dieldrin, diflubenzuron, dimefluthrin, dimehypo, dimethoate, dinotefuran, diofenolan, emamectin, endosulfan, esfenvalerate, ethiprole, etofenprox, etoxazole, fenbutatin oxide, fenitrothion, fenothiocarb, fenoxycarb, fenpropathrin, fenvalerate, fipronil, flometoquin (2-ethyl-3,7-dimethyl-6-[4-(trifluoromethoxy)phenoxy]-4-quinolinyl methyl carbonate), flonicamid, flubendiamide, flucythrinate, flufenerim, flufenoxuron, flufenoxystrobin (methyl (αE)-2-[[2-chloro-4-(trifluoromethyl)phenoxy]methyl]-α-(methoxymethylene)benzeneacetate), flufensulfone (5-chloro-2-[(3,4,4-trifluoro-3-buten-1-yl)sulfonyl]thiazole), fluhexafon, fluopyram, flupiprole (1-[2,6-dichloro-4-(trifluoromethyl)phenyl]-5-[(2-methyl-2-propen-1-yl)amino]-4-[(trifluoromethyl)sulfinyl]-1H-pyrazole-3-carbonitrile), flupyradifurone (4-[[(6-chloro-3-pyridinyl)methyl](2,2-difluoroethyl)amino]-2(5H)-furanone), fluvalinate, tau-fluvalinate, fonophos, formetanate, fosthiazate, halofenozide, heptafluthrin ([2,3,5,6-tetrafluoro-4-(methoxymethyl)phenyl]methyl 2,2-dimethyl-3-[(1Z)-3,3,3-trifluoro-1-propen-1-yl]cyclopropanecarboxylate), hexaflumuron, hexythiazox, hydramethylnon, imidacloprid, indoxacarb, insecticidal soaps, isofenphos, lufenuron, malathion, meperfluthrin ([2,3,5,6-tetrafluoro-4-(methoxymethyl)phenyl]methyl (1R,3S)-3-(2,2-dichloroethenyl)-2,2-dimethylcyclopropanecarboxylate), metaflumizone, metaldehyde, methamidophos, methidathion, methiodicarb, methomyl, methoprene, methoxychlor, metofluthrin, methoxyfenozide, metofluthrin, monocrotophos, monofluorothrin ([2,3,5,6-tetrafluoro-4-(methoxymethyl)phenyl]methyl 3-(2-cyano-1-propen-1-yl)-2,2-dimethylcyclopropanecarboxylate), nicotine, nitenpyram, nithiazine, novaluron, noviflumuron, oxamyl, parathion, parathion-methyl, permethrin, phorate, phosalone, phosmet, phosphamidon, pirimicarb, profenofos, profluthrin, propargite, protrifenbute, pyflubumide (1,3,5-trimethyl-N-(2-methyl-1-oxopropyl)-N-[3-(2-methylpropyl)-4-[2,2,2-trifluoro-1-methoxy-1-(trifluoromethyl)ethyl]phenyl]-1H-pyrazole-4-carboxamide), pymetrozine, pyrafluprole, pyrethrin, pyridaben, pyridalyl, pyrifluquinazon, pyriminostrobin (methyl (αE)-2-[[[2-[(2,4-dichlorophenyl)amino]-6-(trifluoromethyl)-4-pyrimidinyl]oxy]methyl]-α-(methoxymethylene)benzeneacetate), pyriprole, pyriproxyfen, rotenone, ryanodine, silafluofen, spinetoram, spinosad, spirodiclofen, spiromesifen, spirotetramat, sulprofos, sulfoxaflor (N-[methyloxido[1-[6-(trifluoromethyl)-3-pyridinyl]ethyl]-λ⁴-sulfanylidene]cyanamide), tebufenozide, tebufenpyrad, teflubenzuron, tefluthrin, terbufos, tetrachlorvinphos, tetramethrin, tetramethylfluthrin ([2,3,5,6-tetrafluoro-4-(methoxymethyl)phenyl]methyl 2,2,3,3-tetramethylcyclopropanecarboxylate), tetraniliprole, thiacloprid, thiamethoxam, thiodicarb, thiosultap-sodium, tioxazafen (3-phenyl-5-(2-thienyl)-1,2,4-oxadiazole), tolfenpyrad, tralomethrin, triazamate, trichlorfon, triflumezopyrim (2,4-dioxo-1-(5-pyrimidinylmethyl)-3-[3-(trifluoromethyl)phenyl]-2H-pyrido[1,2-a]pyrimidinium inner salt), triflumuron, Bacillus thuringiensis delta-endotoxins, entomopathogenic bacteria, entomopathogenic viruses and entomopathogenic fungi.

Of note are insecticides such as abamectin, acetamiprid, acrinathrin, afidopyropen, amitraz, avermectin, azadirachtin, benfuracarb, bensultap, bifenthrin, buprofezin, cadusafos, carbaryl, cartap, chlorantraniliprole, chlorfenapyr, chlorpyrifos, clothianidin, cyantraniliprole, cyclaniliprole, cycloprothrin, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, dieldrin, dinotefuran, diofenolan, emamectin, endosulfan, esfenvalerate, ethiprole, etofenprox, etoxazole, fenitrothion, fenothiocarb, fenoxycarb, fenvalerate, fipronil, flometoquin, flonicamid, flubendiamide, flufenoxuron, flufenoxystrobin, flufensulfone, flupiprole, flupyradifurone, fluvalinate, formetanate, fosthiazate, heptafluthrin, hexaflumuron, hydramethylnon, imidacloprid, indoxacarb, lufenuron, meperfluthrin, metaflumizone, methiodicarb, methomyl, methoprene, methoxyfenozide, metofluthrin, monofluorothrin, nitenpyram, nithiazine, novaluron, oxamyl, pyflubumide, pymetrozine, pyrethrin, pyridaben, pyridalyl, pyriminostrobin, pyriproxyfen, ryanodine, spinetoram, spinosad, spirodiclofen, spiromesifen, spirotetramat, sulfoxaflor, tebufenozide, tetramethrin, tetramethylfluthrin, thiacloprid, thiamethoxam, thiodicarb, thiosultap-sodium, tralomethrin, triazamate, triflumezopyrim, triflumuron, Bacillus thuringiensis delta-endotoxins, all strains of Bacillus thuringiensis and all strains of nucleo polyhedrosis viruses.

One embodiment of biological agents for mixing with compounds of this invention include entomopathogenic bacteria such as Bacillus thuringiensis, and the encapsulated delta-endotoxins of Bacillus thuringiensis such as MVP® and MVPII® bioinsecticides prepared by the CellCap® process (CellCap®, MVP® and MVPII® are trademarks of Mycogen Corporation, Indianapolis, Ind., USA); entomopathogenic fungi such as green muscardine fungus; and entomopathogenic (both naturally occurring and genetically modified) viruses including baculovirus, nucleopolyhedro virus (NPV) such as Helicoverpa zea nucleopolyhedrovirus (HzNPV), Anagrapha falcifera nucleopolyhedrovirus (AfNPV); and granulosis virus (GV) such as Cydia pomonella granulosis virus (CpGV).

Of particular note is such a combination where the other invertebrate pest control active ingredient belongs to a different chemical class or has a different site of action than the compound of Formula 1. In certain instances, a combination with at least one other invertebrate pest control active ingredient having a similar spectrum of control but a different site of action will be particularly advantageous for resistance management. Thus, a composition of the present invention can further comprise a biologically effective amount of at least one additional invertebrate pest control active ingredient having a similar spectrum of control but belonging to a different chemical class or having a different site of action. These additional biologically active compounds or agents include, but are not limited to, acetylcholinesterase (AChE) inhibitors such as the carbamates methomyl, oxamyl, thiodicarb, triazamate, and the organophosphates chlorpyrifos; GABA-gated chloride channel antagonists such as the cyclodienes dieldrin and endosulfan, and the phenylpyrazoles ethiprole and fipronil; sodium channel modulators such as the pyrethroids bifenthrin, cyfluthrin, beta-cyfluthrin, cyhalothrin, lambda-cyhalothrin, cypermethrin, deltamethrin, dimefluthrin, esfenvalerate, metofluthrin and profluthrin; nicotinic acetylcholinereceptor (nAChR) agonists such as the neonicotinoids acetamiprid, clothianidin, dinotefuran, imidacloprid, nitenpyram, nithiazine, thiacloprid, and thiamethoxam, and sulfoxaflor; nicotinic acetylcholine receptor (nAChR) allosteric activators such as the spinosyns spinetoram and spinosad; chloride channel activators such as the avermectins abamectin and emamectin; juvenile hormone mimics such as diofenolan, methoprene, fenoxycarb and pyriproxyfen; selective homopteran feeding blockers such as pymetrozine and flonicamid; mite growth inhibitors such as etoxazole; inhibitors of mitochondrial ATP synthase such as propargite; ucouplers of oxidative phosphorylation via disruption of the proton gradient such as chlorfenapyr; nicotinic acetylcholine receptor (nAChR) channel blockers such as the nereistoxin analogs cartap; inhibitors of chitin biosynthesis such as the benzoylureas flufenoxuron, hexaflumuron, lufenuron, novaluron, noviflumuron and triflumuron, and buprofezin; dipteran moulting disrupters such as cyromazine; ecdysone receptor agonists such as the diacylhydrazines methoxyfenozide and tebufenozide; octopamine receptor agonists such as amitraz; mitochondrial complex III electron transport inhibitors such as hydramethylnon; mitochondrial complex I electron transport inhibitors such as pyridaben; voltage-dependent sodium channel blockers such as indoxacarb; inhibitors of acetyl CoA carboxylase such as the tetronic and tetramic acids spirodiclofen, spiromesifen and spirotetramat; mitochondrial complex II electron transport inhibitors such as the B-ketonitriles cyenopyrafen and cyflumetofen; ryanidine receptor modulators such as the anthranilic diamides chlorantraniliprole, cyantraniliprole and cyantraniliprole, diamides such as flubendiamide, and ryanodine receptor ligands such as ryanodine; compounds wherein the target site responsible for biological activity is unknown or uncharacterized such as azadirachtin, bifenazate, pyridalyl, pyrifluquinazon and triflumezopyrim; microbial disrupters of insect midgut membranes such as Bacillus thuringensis and the delta-endotoxins they produce and Bacillus sphaericus; and biological agents including nucleo polyhedro viruses (NPV) and other naturally occurring or genetically modified insecticidal viruses.

Further examples of biologically active compounds or agents with which compounds of this invention can be formulated are: fungicides such as acibenzolar-S-methyl, aldimorph, ametoctradin, amisulbrom, anilazine, azaconazole, azoxystrobin, benalaxyl (including benalaxyl-M), benodanil, benomyl, benthiavalicarb (including benthiavalicarb-isopropyl), benzovindiflupyr, bethoxazin, binapacryl, biphenyl, bitertanol, bixafen, blasticidin-S, boscalid, bromuconazole, bupirimate, buthiobate, carboxin, carpropamid, captafol, captan, carbendazim, chloroneb, chlorothalonil, chlozolinate, copper hydroxide, copper oxychloride, copper sulfate, coumoxystrobin, cyazofamid, cyflufenamid, cymoxanil, cyproconazole, cyprodinil, dichlofluanid, diclocymet, diclomezine, dicloran, diethofencarb, difenoconazole, diflumetorim, dimethirimol, dimethomorph, dimoxystrobin, diniconazole (including diniconazole-M), dinocap, dithianon, dithiolanes, dodemorph, dodine, econazole, etaconazole, edifenphos, enoxastrobin (also known as enestroburin), epoxiconazole, ethaboxam, ethirimol, etridiazole, famoxadone, fenamidone, fenaminstrobin, fenarimol, fenbuconazole, fenfuram, fenhexamide, fenoxanil, fenpiclonil, fenpropidin, fenpropimorph, fenpyrazamine, fentin acetate, fentin hydroxide, ferbam, ferimzone, flometoquin, fluazinam, fludioxonil, flufenoxystrobin, flumorph, fluopicolide, fluopyram, fluoxastrobin, fluquinconazole, flusilazole, flusulfamide, flutianil, flutolanil, flutriafol, fluxapyroxad, folpet, fthalide (also known as phthalide), fuberidazole, furalaxyl, furametpyr, hexaconazole, hymexazole, guazatine, imazalil, imibenconazole, iminoctadine albesilate, iminoctadine triacetate, iodicarb, ipconazole, isofetamid, iprobenfos, iprodione, iprovalicarb, isoprothiolane, isopyrazam, isotianil, kasugamycin, kresoxim-methyl, mancozeb, mandipropamid, mandestrobin, maneb, mapanipyrin, mepronil, meptyldinocap, metalaxyl (including metalaxyl-M/mefenoxam), metconazole, methasulfocarb, metiram, metominostrobin, metrafenone, myclobutanil, naftitine, neo-asozin (ferric methanearsonate), nuarimol, octhilinone, ofurace, orysastrobin, oxadixyl, oxathiapiprolin, oxolinic acid, oxpoconazole, oxycarboxin, oxytetracycline, penconazole, pencycuron, penflufen, penthiopyrad, perfurazoate, phosphorous acid (including salts thereof, e.g., fosetyl-aluminm), picoxystrobin, piperalin, polyoxin, probenazole, prochloraz, procymidone, propamocarb, propiconazole, propineb, proquinazid, prothiocarb, prothioconazole, pyraclostrobin, pyrametostrobin, pyraoxystrobin, pyrazophos, pyribencarb, pyributacarb, pyrifenox, pyriofenone, perisoxazole, pyrimethanil, pyrifenox, pyrrolnitrin, pyroquilon, quinconazole, quinmethionate, quinoxyfen, quintozene, silthiofam, sedaxane, simeconazole, spiroxamine, streptomycin, sulfur, tebuconazole, tebufloquin, teclofthalam, tecloftalam, tecnazene, terbinafine, tetraconazole, thiabendazole, thifluzamide, thiophanate, thiophanate-methyl, thiram, tiadinil, tolclofos-methyl, tolprocarb, tolyfluanid, triadimefon, triadimenol, triarimol, triazoxide, tribasic copper sulfate, triclopyricarb, tridemorph, trifloxystrobin, triflumizole, trimoprhamide tricyclazole, trifloxystrobin, triforine, triticonazole, uniconazole, validamycin, valifenalate (also known as valifenal), vinclozolin, zineb, ziram, zoxamide and 1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone; nematocides such as fluopyram, spirotetramat, thiodicarb, fosthiazate, abamectin, iprodione, fluensulfone, dimethyl disulfide, tioxazafen, 1,3-dichloropropene (1,3-D), metam (sodium and potassium), dazomet, chloropicrin, fenamiphos, ethoprophos, cadusaphos, terbufos, imicyafos, oxamyl, carbofuran, tioxazafen, Bacillus firmus and Pasteuria nishizawae; bactericides such as streptomycin; acaricides such as amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben and tebufenpyrad.

In certain instances, combinations of a compound of this invention with other biologically active (particularly invertebrate pest control) compounds or agents (i.e. active ingredients) can result in a greater-than-additive (i.e. enhanced) effect. Reducing the quantity of active ingredients released in the environment while ensuring effective pest control is always desirable. When enhancement of invertebrate pest control active ingredients occurs at application rates giving agronomically satisfactory levels of invertebrate pest control, such combinations can be advantageous for reducing crop production cost and decreasing environmental load.

Compounds of this invention and compositions thereof can be applied to plants genetically transformed to express proteins toxic to invertebrate pests (such as Bacillus thuringiensis delta-endotoxins). Such an application may provide a broader spectrum of plant protection and be advantageous for resistance management. The effect of the exogenously applied invertebrate pest control compounds of this invention may be enhanced with the expressed toxin proteins.

General references for these agricultural protectants (i.e. insecticides, fungicides, nematocides, acaricides, herbicides and biological agents) include The Pesticide Manual, 13th Edition, C. D. S. Tomlin, Ed., British Crop Protection Council, Farnham, Surrey, U. K., 2003 and The BioPesticide Manual, 2^{nd} Edition, L. G. Copping, Ed., British Crop Protection Council, Farnham, Surrey, U. K., 2001.

For embodiments where one or more of these various mixing partners are used, the weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components can expand the spectrum of invertebrate pests controlled beyond the spectrum controlled by the compound of Formula 1 alone.

Invertebrate pests are controlled in agronomic and nonagronomic applications by applying one or more compounds of this invention, typically in the form of a composition, in a biologically effective amount, to the environment of the pests, including the agronomic and/or nonagronomic locus of infestation, to the area to be protected, or directly on the pests to be controlled.

Thus the present invention comprises a method for controlling an invertebrate pest in agronomic and/or nonagronomic applications, comprising contacting the invertebrate pest or its environment with a biologically effective amount of one or more of the compounds of the invention, or with a composition comprising at least one such compound or a composition comprising at least one such compound and a biologically effective amount of at least one additional biologically active compound or agent. Examples of suitable compositions comprising a compound of the invention and a biologically effective amount of at least one additional biologically active compound or agent include granular compositions wherein the additional active compound is present on the same granule as the compound of the invention or on granules separate from those of the compound of the invention.

To achieve contact with a compound or composition of the invention to protect a field crop from invertebrate pests, the compound or composition is typically applied to the seed of the crop before planting, to the foliage (e.g., leaves, stems, flowers, fruits) of crop plants, or to the soil or other growth medium before or after the crop is planted.

One embodiment of a method of contact is by spraying. Alternatively, a granular composition comprising a compound of the invention can be applied to the plant foliage or the soil. Compounds of this invention can also be effectively delivered through plant uptake by contacting the plant with a composition comprising a compound of this invention applied as a soil drench of a liquid formulation, a granular formulation to the soil, a nursery box treatment or a dip of transplants. Of note is a composition of the present invention in the form of a soil drench liquid formulation. Also of note is a method for controlling an invertebrate pest comprising contacting the invertebrate pest or its environment with a biologically effective amount of a compound of the present invention or with a composition comprising a biologically effective amount of a compound of the present invention. Of further note is this method wherein the environment is soil and the composition is applied to the soil as a soil drench formulation. Of further note is that compounds of this invention are also effective by localized application to the locus of infestation. Other methods of contact include application of a compound or a composition of the invention by direct and residual sprays, aerial sprays, gels, seed coatings, microencapsulations, systemic uptake, baits, ear tags, boluses, foggers, fumigants, aerosols, dusts and many others. One embodiment of a method of contact is a dimensionally stable fertilizer granule, stick or tablet comprising a compound or composition of the invention. The compounds of this invention can also be impregnated into materials for fabricating invertebrate control devices (e.g., insect netting).

Compounds of the invention are useful in treating all plants, plant parts and seeds. Plant and seed varieties and cultivars can be obtained by conventional propagation and breeding methods or by genetic engineering methods. Genetically modified plants or seeds (transgenic plants or seeds) are those in which a heterologous gene (transgene) has been stably integrated into the plant's or seed's genome. A transgene that is defined by its particular location in the plant genome is called a transformation or transgenic event.

Genetically modified plant and seed cultivars which can be treated according to the invention include those that are resistant against one or more biotic stresses (pests such as nematodes, insects, mites, fungi, etc.) or abiotic stresses (drought, cold temperature, soil salinity, etc.), or that contain other desirable characteristics. Plants and seeds can be genetically modified to exhibit traits of, for example, herbicide tolerance, insect-resistance, modified oil profiles or drought tolerance.

Treatment of genetically modified plants and seeds with compounds of the invention may result in super-additive or enhanced effects. For example, reduction in application rates, broadening of the activity spectrum, increased tolerance to biotic/abiotic stresses or enhanced storage stability may be greater than expected from just simple additive effects of the application of compounds of the invention on genetically modified plants and seeds.

Compounds of this invention are also useful in seed treatments for protecting seeds from invertebrate pests. In the context of the present disclosure and claims, treating a seed means contacting the seed with a biologically effective amount of a compound of this invention, which is typically formulated as a composition of the invention. This seed treatment protects the seed from invertebrate soil pests and generally can also protect roots and other plant parts in contact with the soil of the seedling developing from the germinating seed. The seed treatment may also provide protection of foliage by translocation of the compound of this invention or a second active ingredient within the developing plant. Seed treatments can be applied to all types of seeds, including those from which plants genetically transformed to express specialized traits will germinate. Representative examples include those expressing proteins toxic to invertebrate pests, such as Bacillus thuringiensis toxin or those expressing herbicide resistance such as glyphosate acetyltransferase, which provides resistance to glyphosate. Seed treatments with compounds of this invention can also increase vigor of plants growing from the seed.

One method of seed treatment is by spraying or dusting the seed with a compound of the invention (i.e. as a formulated composition) before sowing the seeds. Compositions formulated for seed treatment generally comprise a film former or adhesive agent. Therefore typically a seed coating composition of the present invention comprises a biologically effective amount of a compound of Formula 1, an N-oxide or salt thereof, and a film former or adhesive agent. Seed can be coated by spraying a flowable suspension concentrate directly into a tumbling bed of seeds and then drying the seeds. Alternatively, other formulation types such as wetted powders, solutions, suspoemulsions, emulsifiable concentrates and emulsions in water can be sprayed on the seed. This process is particularly useful for applying film coatings on seeds. Various coating machines and processes are available to one skilled in the art. Suitable processes include those listed in P. Kosters et al., Seed Treatment: Progress and Prospects, 1994 BCPC Mongraph No. 57, and references listed therein.

Compounds of Formula 1 and their compositions, both alone and in combination with other insecticides, nematicides, and fungicides, are particularly useful in seed treatment for crops including, but not limited to, maize or corn, soybeans, cotton, cereal (e.g., wheat, oats, barley, rye and rice), potatoes, vegetables and oilseed rape.

Other insecticides with which compounds of Formula 1 can be formulated to provide mixtures useful in seed treatment include abamectin, acetamiprid, acrinathrin, amitraz, avermectin, azadirachtin, bensultap, bifenthrin, buprofezin, cadusafos, carbaryl, carbofuran, cartap, chlorantraniliprole, chlorfenapyr, chlorpyrifos, clothianidin, cyantraniliprole, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, dieldrin, dinotefuran, diofenolan, emamectin, endosulfan, esfenvalerate, ethiprole, etofenprox, etoxazole, fenothiocarb, fenoxycarb, fenvalerate, fipronil, flonicamid, flubendiamide, flufenoxuron, fluvalinate, formetanate, fosthiazate, hexaflumuron, hydramethylnon, imidacloprid, indoxacarb, lufenuron, metaflumizone, methiocarb, methomyl, methoprene, methoxyfenozide, nitenpyram, nithiazine, novaluron, oxamyl, pymetrozine, pyrethrin, pyridaben, pyridalyl, pyriproxyfen, ryanodine, spinetoram, spinosad, spirodiclofen, spiromesifen, spirotetramat, sulfoxaflor, tebufenozide, tetramethrin, thiacloprid, thiamethoxam, thiodicarb, thiosultap-sodium, tralomethrin, triazamate, triflumuron, Bacillus thuringiensis delta-endotoxins, all strains of Bacillus thuringiensis and all strains of nucleo polyhedrosis viruses.

Fungicides with which compounds of Formula 1 can be formulated to provide mixtures useful in seed treatment include amisulbrom, azoxystrobin, boscalid, carbendazim, carboxin, cymoxanil, cyproconazole, difenoconazole, dimethomorph, fluazinam, fludioxonil, fluquinconazole, fluopicolide, fluoxastrobin, flutriafol, fluxapyroxad, ipconazole, iprodione, metalaxyl, mefenoxam, metconazole, myclobutanil, paclobutrazole, penflufen, picoxystrobin, prothioconazole, pyraclostrobin, sedaxane, silthiofam, tebuconazole, thiabendazole, thiophanate-methyl, thiram, trifloxystrobin and triticonazole.

Compositions comprising compounds of Formula 1 useful for seed treatment can further comprise bacteria and fungi that have the ability to provide protection from the harmful effects of plant pathogenic fungi or bacteria and/or soil born animals such as nematodes. Bacteria exhibiting nematicidal properties may include but are not limited to Bacillus firmus, Bacillus cereus, Bacillius subtiliis and Pasteuria penetrans. A suitable Bacillus firmus strain is strain CNCM I-1582 (GB-126) which is commercially available as BioNem™. A suitable Bacillus cereus strain is strain NCMM I-1592. Both Bacillus strains are disclosed in U.S. Pat. No. 6,406,690. Other suitable bacteria exhibiting nematicidal activity are B. amyloliquefaciens IN937a and B. subtilis strain GB03. Bacteria exhibiting fungicidal properties may include but are not limited to B. pumilus strain GB34. Fungal species exhibiting nematicidal properties may include but are not limited to Myrothecium verrucaria, Paecilomyces lilacinus and Purpureocillium lilacinum.

Seed treatments can also include one or more nematicidal agents of natural origin such as the elicitor protein called harpin which is isolated from certain bacterial plant pathogens such as Erwinia amylovora. An example is the Harpin-N-Tek seed treatment technology available as N-Hibit™ Gold CST.

Seed treatments can also include one or more species of legume-root nodulating bacteria such as the microsymbiotic nitrogen-fixing bacteria Bradyrhizobium japonicum. These inocculants can optionally include one or more lipo-chitooligosaccharides (LCOs), which are nodulation (Nod) factors produced by rhizobia bacteria during the initiation of nodule formation on the roots of legumes. For example, the Optimize® brand seed treatment technology incorporates LCO Promoter Technology™ in combination with an inocculant.

Seed treatments can also include one or more isoflavones which can increase the level of root colonization by mycorrhizal fungi. Mycorrhizal fungi improve plant growth by enhancing the root uptake of nutrients such as water, sulfates, nitrates, phosphates and metals. Examples of isoflavones include, but are not limited to, genistein, biochanin A, formononetin, daidzein, glycitein, hesperetin, naringenin and pratensein. Formononetin is available as an active ingredient in mycorrhizal inocculant products such as PHC Colonize® AG.

Seed treatments can also include one or more plant activators that induce systemic acquired resistance in plants following contact by a pathogen. An example of a plant activator which induces such protective mechanisms is acibenzolar-S-methyl.

The treated seed typically comprises a compound of the present invention in an amount from about 0.1 g to 1 kg per 100 kg of seed (i.e. from about 0.0001 to 1% by weight of the seed before treatment). A flowable suspension formulated for seed treatment typically comprises from about 0.5 to about 70% of the active ingredient, from about 0.5 to about 30% of a film-forming adhesive, from about 0.5 to about 20% of a dispersing agent, from 0 to about 5% of a thickener, from 0 to about 5% of a pigment and/or dye, from 0 to about 2% of an antifoaming agent, from 0 to about 1% of a preservative, and from 0 to about 75% of a volatile liquid diluent.

The compounds of this invention can be incorporated into a bait composition that is consumed by an invertebrate pest or used within a device such as a trap, bait station, and the like. Such a bait composition can be in the form of granules which comprise (a) active ingredients, namely a biologically effective amount of a compound of Formula 1, an N-oxide, or salt thereof; (b) one or more food materials; optionally (c) an attractant, and optionally (d) one or more humectants. Of note are granules or bait compositions which comprise between about 0.001-5% active ingredients, about 40-99% food material and/or attractant; and optionally about 0.05-10% humectants, which are effective in controlling soil invertebrate pests at very low application rates, particularly at doses of active ingredient that are lethal by ingestion rather than by direct contact. Some food materials can function both as a food source and an attractant. Food materials include carbohydrates, proteins and lipids. Examples of food materials are vegetable flour, sugar, starches, animal fat, vegetable oil, yeast extracts and milk solids. Examples of attractants are odorants and flavorants, such as fruit or plant extracts, perfume, or other animal or plant component, pheromones or other agents known to attract a target invertebrate pest. Examples of humectants, i.e. moisture retaining agents, are glycols and other polyols, glycerine and sorbitol. Of note is a bait composition (and a method utilizing such a bait composition) used to control at least one invertebrate pest selected from the group consisting of ants, termites and cockroaches. A device for controlling an invertebrate pest can comprise the present bait composition and a housing adapted to receive the bait composition, wherein the housing has at least one opening sized to permit the invertebrate pest to pass through the opening so the invertebrate pest can gain access to the bait composition from a location outside the housing, and wherein the housing is further adapted to be placed in or near a locus of potential or known activity for the invertebrate pest.

One embodiment of the present invention relates to a method for controlling invertebrate pests, comprising diluting the pesticidal composition of the present invention (a compound of Formula 1 formulated with surfactants, solid diluents and liquid diluents or a formulated mixture of a compound of Formula 1 and at least one other pesticide) with water, and optionally adding an adjuvant to form a diluted composition, and contacting the invertebrate pest or its environment with an effective amount of said diluted composition.

Although a spray composition formed by diluting with water a sufficient concentration of the present pesticidal composition can provide sufficient efficacy for controlling invertebrate pests, separately formulated adjuvant products can also be added to spray tank mixtures. These additional adjuvants are commonly known as “spray adjuvants” or “tank-mix adjuvants”, and include any substance mixed in a spray tank to improve the performance of a pesticide or alter the physical properties of the spray mixture. Adjuvants can be surfactants, emulsifying agents, petroleum-based crop oils, crop-derived seed oils, acidifiers, buffers, thickeners or defoaming agents. Adjuvants are used to enhancing efficacy (e.g., biological availability, adhesion, penetration, uniformity of coverage and durability of protection), or minimizing or eliminating spray application problems associated with incompatibility, foaming, drift, evaporation, volatilization and degradation. To obtain optimal performance, adjuvants are selected with regard to the properties of the active ingredient, formulation and target (e.g., crops, insect pests).

Among the spray adjuvants, oils including crop oils, crop oil concentrates, vegetable oil concentrates and methylated seed oil concentrates are most commonly used to improve the efficacy of pesticides, possibly by means of promoting more even and uniform spray deposits. In situations where phytotoxicity potentially caused by oils or other water-immiscible liquids are of concern, spray compositions prepared from the composition of the present invention will generally not contain oil-based spray adjuvants. However, in situations where phytotoxicity caused by oil-based spray adjuvants is commercially insignificant, spray compositions prepared from the composition of the present composition can also contain oil-based spray adjuvants, which can potentially further increase control of invertebrate pests, as well as rainfastness.

Products identified as “crop oil” typically contain 95 to 98% paraffin or naphtha-based petroleum oil and 1 to 2% of one or more surfactants functioning as emulsifiers. Products identified as “crop oil concentrates” typically consist of 80 to 85% of emulsifiable petroleum-based oil and 15 to 20% of nonionic surfactants. Products correctly identified as “vegetable oil concentrates” typically consist of 80 to 85% of vegetable oil (i.e. seed or fruit oil, most commonly from cotton, linseed, soybean or sunflower) and 15 to 20% of nonionic surfactants. Adjuvant performance can be improved by replacing the vegetable oil with methyl esters of fatty acids that are typically derived from vegetable oils. Examples of methylated seed oil concentrates include MSO® Concentrate (UAP-Loveland Products, Inc.) and Premium MSO Methylated Spray Oil (Helena Chemical Company).

The amount of adjuvants added to spray mixtures generally does not exceed about 2.5% by volume, and more typically the amount is from about 0.1 to about 1% by volume. The application rates of adjuvants added to spray mixtures are typically between about 1 to 5 L per hectare. Representative examples of spray adjuvants include: Adigor® (Syngenta) 47% methylated rapeseed oil in liquid hydrocarbons, Silwet® (Helena Chemical Company) polyalkyleneoxide modified heptamethyltrisiloxane and Assist® (BASF) 17% surfactant blend in 83% paraffin based mineral oil.

The compounds of this invention can be applied without other adjuvants, but most often application will be of a formulation comprising one or more active ingredients with suitable carriers, diluents, and surfactants and possibly in combination with a food depending on the contemplated end use. One method of application involves spraying a water dispersion or refined oil solution of a compound of the present invention. Combinations with spray oils, spray oil concentrations, spreader stickers, adjuvants, other solvents, and enhancing agents such as piperonyl butoxide often enhance compound efficacy. For nonagronomic uses such sprays can be applied from spray containers such as a can, a bottle or other container, either by means of a pump or by releasing it from a pressurized container, e.g., a pressurized aerosol spray can.

Such spray compositions can take various forms, for example, sprays, mists, foams, fumes or fog. Such spray compositions thus can further comprise propellants, foaming agents, etc. as the case may be. Of note is a spray composition comprising a biologically effective amount of a compound or a composition of the present invention and a carrier. One embodiment of such a spray composition comprises a biologically effective amount of a compound or a composition of the present invention and a propellant. Representative propellants include, but are not limited to, methane, ethane, propane, butane, isobutane, butene, pentane, isopentane, neopentane, pentene, hydrofluorocarbons, chlorofluorocarbons, dimethyl ether, and mixtures of the foregoing. Of note is a spray composition (and a method utilizing such a spray composition dispensed from a spray container) used to control at least one invertebrate pest selected from the group consisting of mosquitoes, black flies, stable flies, deer flies, horse flies, wasps, yellow jackets, hornets, ticks, spiders, ants, gnats, and the like, including individually or in combinations.

The following Tests demonstrate the control efficacy of compounds of this invention on specific pests. “Control efficacy” represents inhibition of invertebrate pest development (including mortality) that causes significantly reduced feeding. The pest control protection afforded by the compounds is not limited, however, to these species. See Index Table A below for compound descriptions. The following abbreviations are used in Index Table A: i-Pr means iso-propyl, c-Pr means cyclopropyl and Ph means phenyl. The abbreviation “Cmpd. No.” stands for “Compound Number”, and the abbreviation “Ex.” stands for “Example” and is followed by a number indicating in which example the compound is prepared. The abbreviation “m.p.” stands for melting point.

INDEX TABLE A

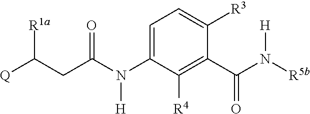

| Cmpd. No. | Q | R^{1a} | R³ | R⁴ | R^{5b} | m.p. (° C.) |
|---|---|---|---|---|---|---|
| 1 | 3,4-di-Cl—Ph | CF₃ | Cl | H | 4-F—Ph | 119-120 |
| 2 | 3-CF₃—Ph | CF₃ | Cl | H | CH₂CF₃ | 84-85 |
| 3 | 3,5-di-Cl—Ph | CF₃ | Cl | H | c-Pr | 192-196 |
| 4 | 3-CF₃—Ph | CF₃ | Cl | H | 3-CH₃—Ph | * |
| 5 (Ex. 1) | 3-CF₃—Ph | CF₃ | Cl | H | 2,4-di-F—Ph | 158-159 |
| 6 | 3-CF₃—Ph | CF₃ | Cl | H | 2-CH₃-4-F—Ph | 174-175 |
| 7 | 3-CF₃—Ph | CF₃ | Cl | H | 3-CF₃—Ph | 56-57 |
| 8 | 3-CF₃—Ph | CF₃ | Cl | H | 4-CF₃—Ph | * |
| 9 | 3-CF₃—Ph | CF₃ | F | H | c-Pr | 133-134 |
| 10 | 3-CF₃—Ph | CF₃ | F | H | 4-F—Ph | 114-115 |
| 11 | 3-CF₃—Ph | CF₃ | F | F | 4-F—Ph | 193-194 |
| 12 | 3-CF₃—Ph | CF₃ | F | F | c-Pr | 164-165 |
| 13 | 3,4-di-Cl—Ph | CF₃ | F | H | 4-F—Ph | 155-156 |
| 14 | 3,4-di-Cl—Ph | CF₃ | F | H | c-Pr | 109-110 |
| 15 | 3-CF₃—Ph | CF₃ | F | F | CH(CH₃)C≡N | * |
| 16 | 3,5-di-Cl—Ph | CF₃ | F | H | c-Pr | 162-163 |
| 17 (Ex. 2) | 3,5-di-Cl—Ph | CF₃ | F | H | 4-F—Ph | 181-182 |
| 18 | 3,5-di-Cl—Ph | CF₃ | H | H | CH₂CF₃ | 221-225 |
| 19 | 3,5-di-Cl—Ph | CF₃ | Br | H | c-Pr | 193-194 |
| 20 | 3,4-di-Cl—Ph | CF₃ | Cl | H | c-Pr | 168-169 |
| 21 | 3,4-di-Cl—Ph | CF₃ | Cl | H | CH₂CF₃ | 59-60 |
| 22 | 3,4-di-Cl—Ph | CF₃ | C | H | c-Pr | 198-199 |
| 23 | 3,5-di-Cl—Ph | CF₃ | H | H | c-Pr | 194-198 |
| 24 | 3,5-di-Cl—Ph | CF₃ | Cl | H | CH₂CF₃ | 195-199 |
| 25 | 3-CF₃—Ph | CF₃ | F | F | 5-F-2-pyridinyl | 136-137 |
| 26 | Ph | CF₃ | H | F | 4-CF₃—Ph | 123-124 |
| 27 | 3-Cl-5-F—Ph | CF₃ | Cl | H | 5-CF₃-2-pyridinyl | * |
| 28 | 3,4-di-Cl—Ph | CF₃ | Cl | H | 2-Cl-4-CF₃—Ph | * |
| 29 | 3,5-di-F—Ph | CF₃ | Cl | H | 5-CF₃-2-pyridinyl | * |
| 30 | 3,4-di-Cl—Ph | CF₃ | Cl | H | 5-Cl-2-pyridinyl | * |
| 31 | 3,5-di-F—Ph | CF₃ | Cl | H | 5-Cl-2-pyridinyl | 114-115 |
| 32 | 3,5-di-Cl—Ph | CF₃ | Cl | H | 3-F—Ph | * |
| 33 | 3,5-di-Cl—Ph | CF₃ | Cl | H | 2-F—Ph | * |
| 34 | 3,5-di-Cl—Ph | CF₃ | Cl | H | 4-CF₃—Ph | 54-55 |
| 35 | 3,4-di-Cl—Ph | CF₃ | Cl | H | 3-F—Ph | 79-80 |
| 36 | 3,4-di-Cl—Ph | CF₃ | Cl | H | 2-F—Ph | 75-76 |
| 37 | 3,5-di-Cl—Ph | CF₃ | Cl | H | CH₂(C═O)NHCH₂CH₃ | 102-106 |
| 38 | 3-CF₃—Ph | CF₃ | Cl | H | 3-F—Ph | * |
| 39 | 3-CF₃—Ph | CF₃ | Cl | H | 5-F-2-pyridinyl | 129-130 |
| 40 | Ph | CF₃ | Cl | H | 2-Br-4-CF₃—Ph | * |
| 41 | 3-Cl-4-F—Ph | CF₃ | Cl | H | 3-F—Ph | 92-93 |
| 42 | 3-Cl-4-F—Ph | CF₃ | Cl | H | 2-F—Ph | 73-74 |
| 43 | 3-F—Ph | CF₃ | Cl | H | 4-CF₃—Ph | 107-108 |
| 44 | 3-F—Ph | CF₃ | Cl | H | 4-F—Ph | 82-83 |
| 45 | 3-F—Ph | CF₃ | Cl | H | 2,4-di-F—Ph | 143-144 |
| 46 | Ph | CF₃ | Cl | H | 4-F—Ph | 96-97 |
| 47 | Ph | CF₃ | Cl | H | 4-CF₃—Ph | 143-144 |
| 48 | Ph | CF₃ | Cl | H | 2,4-di-F—Ph | 119-120 |
| 49 | 3,5-di-F—Ph | CF₃ | Cl | H | 4-CF₃—Ph | 137-138 |
| 50 | 3,5-di-F—Ph | CF₃ | Cl | H | 4-F—Ph | 103-104 |
| 51 | 3,4-di-Cl—Ph | CF₃ | Cl | H | 4-CF₃—Ph | * |
| 52 | 3,4-di-Cl—Ph | CF₃ | Cl | H | 2,4-di-F—Ph | 115-116 |
| 53 | 3,4-di-Cl—Ph | CF₃ | Cl | H | 5-F-2-pyridinyl | 120-121 |
| 54 | 3,5-di-Cl—Ph | CF₃ | Cl | H | 5-F-2-pyridinyl | 157-158 |
| 55 | 3,5-di-Cl—Ph | CF₃ | Cl | H | 5-Cl-2-pyridinyl | 100-101 |
| 56 | 3,5-di-Cl—Ph | CF₃ | Cl | H | 5-CF₃-2-pyridinyl | * |
| 57 | 3,5-di-Cl—Ph | CF₃ | Cl | H | 2-Cl-4-CF₃—Ph | * |
| 58 | 3,5-di-Cl—Ph | CF₃ | Cl | H | 2-Br-4-CF₃—Ph | * |
| 59 | 3,5-di-Cl—Ph | CF₃ | Cl | H | CH₂(C═O)NHCH₂CH₃ | 163-167 |
| 60 | 3,5-di-Cl—Ph | CF₃ | Cl | H | 2,4-di-F—Ph | 156-160 |
| 61 | 3,5-di-F—Ph | CF₃ | Cl | H | 2,4-di-F—Ph | 143-144 |
| 62 | 3,5-di-F—Ph | CF₃ | Cl | H | 5-F-2-pyridinyl | 119-120 |
| 63 | 3,5-di-Cl—Ph | CF₃ | Cl | H | 2,6-di-F-3-pyridinyl | 89-90 |
| 64 | 3,5-di-Cl—Ph | CF₃ | Cl | H | 2-Cl-6-CF₃-3-pyridinyl | 128-129 |
| 65 | 3,5-di-F—Ph | CF₃ | Cl | H | 2,6-di-F-3-pyridinyl | 67-68 |
| 66 | 3,5-di-F—Ph | CF₃ | Cl | H | 2-Cl-6-CF₃-3-pyridinyl | 57-58 |
| 67 | 3,5-di-Cl—Ph | CF₃ | Cl | H | 6-Cl-3-pyridinyl | 113-114 |
| 68 | 3-Cl-4-F—Ph | CF₃ | Cl | H | 5-F-2-pyridinyl | 111-112 |
| 69 | 3,4-di-Cl—Ph | CF₃ | Cl | H | 2-Cl-6-CF₃-3-pyridinyl | * |
| 70 | Ph | CF₃ | Cl | H | 3-F-5-CF₃-2-pyridinyl | 108-109 |
| 71 [Note 1] | 3,5-di-Cl—Ph | CF₃ | Cl | H | CH₂CF₃ | 150-152 |
| 72 [Note 2] | 3,5-di-Cl—Ph | CF₃ | Cl | H | CH₂CF₃ | 141-145 |
| 73 | 3,5-di-Cl—Ph | CF₃ | Cl | H | CH₂CF₃ | 184-188 |
| 74 | 3,5-di-Cl—Ph | CF₃ | Cl | H | i-Pr | 175-179 |
| 75 | 3,5-di-Cl—Ph | CF₃ | Cl | H | CH₂-c-Pr | 204-208 |
| 76 | 3,5-di-Cl—Ph | CF₃ | Cl | H | 4-F—Ph | 110-114 |
| 77 | 3,4-di-Cl—Ph | CF₃ | F | H | CH₂-c-Pr | 178-179 |
| 78 | 3-CF₃—Ph | CF₃ | Cl | H | 2,6-di-F-3-pyridinyl | 68-69 |
| 79 | 3-CF₃—Ph | CF₃ | Cl | H | 4-F—Ph | 75-76 |
| 80 | 3-CF₃—Ph | CF₃ | Cl | H | 2-F—Ph | 142-143 |
| 81 | 3-CF₃—Ph | CF₃ | Cl | H | 2,4-di-Cl—Ph | 113-115 |
| 82 | 3-CF₃—Ph | CF₃ | Cl | H | 2-Cl-4-F—Ph | 115-116 |
| 83 | 3-CF₃—Ph | CF₃ | Cl | H | 2-F-4-Cl—Ph | 129-130 |
| 84 | 3-CF₃—Ph | CF₃ | Cl | H | 6-Cl-3-pyridinyl | 92-94 |
| 85 | 3-CF₃—Ph | CF₃ | Cl | H | 2,4,6-tri-F—Ph | 103-105 |
| 86 | 3-CF₃—Ph | CF₃ | F | H | 2,4-di-F—Ph | 126-127 |
| 87 | 3-CF₃—Ph | CF₃ | F | H | 2,4-di-Cl—Ph | 155-156 |
| 88 | 3-CF₃—Ph | CF₃ | F | H | 2-Cl-4-F—Ph | 114-115 |
| 89 | 3-CF₃—Ph | CF₃ | F | H | 2,6-di-F-3-pyridinyl | 83-84 |
| 90 | 3,4-di-Cl—Ph | CF₃ | F | H | 2,4-di-F—Ph | 129-130 |
| 91 | 3,4-di-Cl—Ph | CF₃ | F | H | 2,4-di-Cl—Ph | 115-116 |
| 94 | 3,4-di-Cl—Ph | CF3 | Cl | H | 2,4-di-F—Ph | 65-66 |
| 95 | 3,4-di-Cl—Ph | CF3 | Cl | H | 2,4-di-F-3-Pyridyl | 59-60 |
| 96 | 3-CF3—Ph | CF3 | Cl | H | 3,5-di-Cl-2-Pyridyl | 79-80 |
| 97 | 3-CF3—Ph | CF3 | F | F | 2,4-di-F—Ph | 144-145 |
| 98 | 3-CF3—Ph | CF3 | F | F | 2-Cl-4-F—Ph | 126-127 |
| 99 | 3-CF3—Ph | CF3 | F | F | 3,5-di-F-2-Pyridyl | 44-45 |
| 100 | 3-CF3—Ph | CF3 | Cl | H | 3,4-di-F—Ph | 85-86 |
| 101 | 3-CF3—Ph | CF3 | Cl | H | 3,5-di-F-2-Pyridyl | 127-128 |
| 102 | 3-CF3—Ph | CF3 | Cl | H | 4-CN—Ph | * |
| 103 (Note 3) | 3-CF3—Ph | CF3 | Cl | H | 2,4-di-F—Ph | 136-139 |
| 104 (Note 4) | 3-CF3—Ph | CF3 | Cl | H | 2,4-di-F—Ph | 138-141 |
| 105 | 3-Cl-4-F-5-Cl—Ph | CF3 | Cl | H | 2,4-di-F—Ph | 150-153 |
| 106 | 3-CF3—Ph | CF3 | Cl | H | 3-OCH3-4-F—Ph | 106-107 |
| 107 | 3-CF3—Ph | CF3 | Cl | H | 3-Cl-4-F—Ph | 50-60 |
| 108 | 3-CF3—Ph | CF3 | Cl | H | 4-OCF3—Ph | * |
| 109 | 3-CF3—Ph | CF3 | Cl | H | 2,3-di-F—Ph | * |
| 110 | 3-CF3—Ph | CF3 | Cl | H | 3-Br-4-F—Ph | 107-108 |
| 111 | 3-CF3—Ph | CF3 | Cl | H | 3,4,5-tri-F—Ph | 101-102 |
| 112 | 3-CF3—Ph | CF3 | Cl | H | 4-OCH3—Ph | 152-153 |
| 113 | 3-Cl-4-F-5-Cl—Ph | CF3 | Cl | H | 4-F—Ph | 119-122 |
| 114 | 3-Cl-4-F-5-Cl—Ph | CF3 | Cl | H | 2-F—Ph | 165-168 |
| 115 | 3-Cl-4-F—Ph | CF3 | Cl | H | 2,4-di-F—Ph | 88-91 |
| 116 | 3-CF3—Ph | CF3 | Cl | H | 2,4-di-F-Benzyl | 120-123 |
| 117 | 3-CF3—Ph | CF3 | Cl | H | CH2CH2OCH3 | 145-148 |
| 118 | 3-Cl-4-F-5-Cl—Ph | CF3 | Cl | H | 4-F—Ph | 103-107 |
| 119 | 3-CF3—Ph | CF3 | Cl | H | CH2(2-Pyrimidinyl) | 174-177 |
| 120 | 3-CF3—Ph | CF3 | Cl | H | 4-F-CH2(4-F—Ph) | 125-128 |
| 121 | 3-CF3—Ph | CF3 | Cl | H | CH2CH2SCH3 | 96-100 |
| 122 | 3-CF3—Ph | CF3 | Cl | H | CH2CH2SO2CH3 | ** |
| 123 | 3,5-di-Cl—Ph | CF3 | CF3 | H | 2,4-di-F—Ph | 175-179 |
| 124 | 3,5-di-Cl—Ph | CF3 | CF3 | H | 4-F—Ph | 190-194 |
| 125 | 3-CF3—Ph | CF3 | Cl | H | CH2CH2S(O)CH3 | ** |
| 126 | 3,5-di-Cl—Ph | CF3 | CF3 | H | 2-F—Ph | 88-91 |
| 127 | 3,5-di-Cl—Ph | CF3 | CF3 | H | 4-OCF3—Ph | 105-108 |
| 128 | 3,5-di-Cl—Ph | CF3 | CF3 | H | 2,4-di-F-3-Pyridyl | 137-140 |
| 129 | 3,5-di-Cl—Ph | CF3 | CF3 | H | 3,4-di-F—Ph | 135-138 |
| 130 | 3,5-di-Cl—Ph | CF3 | CF3 | H | 3-Cl-4-F—Ph | 151-154 |
| 131 | 3,5-di-Cl—Ph | CF3 | CF3 | H | 3-Cl-4-F—Ph | 113-116 |
| 132 | 3,5-di-Cl—Ph | CF3 | CF3 | H | 4-CN—Ph | 124-127 |
| 133 | 3-CF3—Ph | CF3 | CF3 | H | 4-F—Ph | 114-117 |
| 134 | 3-CF3—Ph | CF3 | CF3 | H | 2-F—Ph | 86-89 |
| 135 | 3-CF3—Ph | CF3 | CF3 | H | 3-Cl-4-F—Ph | 150-153 |
| 136 | 3-CF3—Ph | CF3 | CF3 | H | 3,4-di-F—Ph | 114-117 |
| 137 | 3-CF3—Ph | CF3 | CF3 | H | 4-CF3—Ph | 98-101 |
| 138 | 3-CF3—Ph | CF3 | CF3 | H | 2,4-di-F—Ph | 94-97 |
| 139 | 3-CF3—Ph | CF3 | CF3 | H | 4-OCF3—Ph | 104-107 |
| 140 | 3-CF3—Ph | CF3 | CF3 | H | 4-CN—Ph | 168-171 |
| 141 | 3-CF3—Ph | CF3 | CF3 | H | 2,4-di-F-3-Pyridyl | 162-165 |
| 142 (Note 5) | 3,5-di-CF3—Ph | CF3 | Cl | H | 2,4-di-F—Ph | 77-80 |
| 143 | 3,5-di-CF3—Ph | CF3 | Cl | H | 2,4-di-F—Ph | 71-74 |
| 144 (Note 6) | 3,5-di-CF3—Ph | CF3 | Cl | H | 2,4-di-F—Ph | 86-89 |
| 154 | 3-CF3—Ph | CF2H | Cl | H | 2,4-di-F—Ph | ** |
| 155 | 3-CF3—Ph | CF2H | F | H | 2,4-di-F—Ph | ** |
| 156 | 3,5-di-Cl—Ph | CF2H | Cl | H | 2,4-di-F—Ph | ** |
| 157 | 3,5-di-Cl—Ph | CF2H | F | H | 2,4-di-F—Ph | ** |

INDEX TABLE B

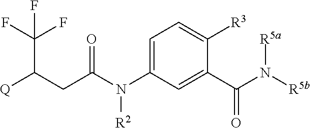

| Cmpd. No. | Q | R² | R³ | R^{5a} | R^{5b} | m.p. (° C.) |
|---|---|---|---|---|---|---|
| 145 | 3-CF3—Ph | CH3 | Cl | H | 2,4-d--F—Ph | ** |
| 146 (Note 7) | 3-CF3—Ph | CH3 | Cl | H | 2,4-d--F—Ph | 60-64 |
| 147 (Note 8) | 3-CF3—Ph | CH3 | Cl | H | 2,4-d--F—Ph | 59-62 |
| 148 | 3-CF3—Ph | H | Cl | CH3 | 2,4-d--F—Ph | 96-99 |

INDEX TABLE C

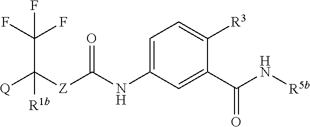

| Cmpd. No. | Q | R² | R³ | R^{5a} | R^{5b} | m.p. (° C.) |
|---|---|---|---|---|---|---|
| 149 | 3,5-di-Cl—Ph | OH | CH2 | Cl | 2,4-di-F—Ph | ** |
| 150 | 3-CF3—Ph | OH | CH2 | Cl | 4-F—Ph | ** |
| 151 | 3-CF3—Ph | H | NH | F | CH2CF3 | 147-150 |
| 152 | 3-CF3—Ph | H | NH | F | 2,4-di-F—Ph | 185-188 |
| 153 | 3,5-di-Cl—Ph | H | NH | F | 2,4-di-F—Ph | 222-225 |

*See Index Table D for ¹H NMR data.
**See Index Table E for MS data.

Note 1: enantiomer (99.24% ee) at R^{1a} [α]_{D}²⁰ −40.4773° (concentration=0.75 in dichloromethane).

Note 2: enantiomer (99.56% ee) at R^{1a} [α]_{D}²⁰+63.8827° (concentration=0.75 in dichloromethane).

Note 3: enantiomer (99.90%) at R1a [a]D20 −35.31 degrees (concentration=0.75 in dichloromethane.

Note 4: enantiomer (99.45%) at R1a [a]D20 +33.32 degrees (concentration=0.75 in dichloromethane).

Note 5: enantiomer at R1a [a]D20 −38.10 degrees (concentration=0.50 in methanol).

Note 6: enantiomer at R1a [a]D20 +44.52 degrees (concentration=0.50 in methanol).

Note 7: enantiomer (99.90%) at R1a [a]D20 +55.92 degrees (concentration=0.5% in methanol).

Note 8: enantiomer (99.90%) at R1a [a]D20 −55.10 degrees (concentration=0.5% in methanol).

INDEX TABLE D

| Cmpd. No. | ¹HNMR Data^{a} |
|---|---|
| 4 | δ 9.03 (s, 1H), 8.27 (s, 1H), 7.63 (d, 1H), 7.54 (d, 2H), 7.45-7.40 (m, 4H), 7.31 |
| | (s, 1H), 7.27 (t, 1H), 7.15 (d, 1H), 7.03 (d, 1H), 4.15 (m, 1H), 3.05 (dd, 1H), 2.9 |
| | (m, 1H), 2.35 (s, 3H). |
| 8 | δ 8.83 (s, 1H), 8.40 (s, 1H), 7.75 (d, 2H), 7.70-7.55 (m, 7H), 7.50 (m, 1H), 7.30 |
| | (d, 1H), 4.15 (m, 1H), 3.15 (m, 1H), 2.95 (m, 1H). |
| 15 | δ 8.00 (m, 2H), 7.65 (m, 3H), 7.50 (m, 1H), 7.00 (m, 1H), 6.80 (t, 1H), 5.05 (q, 1H), |
| | 4.15 (m, 1H), 3.15 (m, 1H), 3.05 (m, 1H), 1.65 (d, 3H). |
| 27 | δ 8.95 (s, 1H), 8.52 (s, 1H), 8.48 (d, 1H), 7.97 (d, 1H), 7.80 (s, 1H), 7.68 (s, 1H), 7.60 |
| | (d, 1H), 7.40 (m, 2H), 7.20 (m, 1H), 7.15 (t, 1H), 4.10 (m, 1H), 3.10 (m, 1H), 2.80- |
| | 2.90 (m, 1H). |
| 28 | δ 8.75 (s, 1H), 8.70 (d, 1H), 7.80 (s, 1H), 7.70 (m, 2H), 7.65 (s, 1H), 7.60 (d, 1H), |
| | 7.40 (m, 3H), 7.20 (d, 1H), 4.05 (m, 1H), 3.10 (dd, 1H), 2.80-2.90 (m, 1H). |
| 29 | δ 9.20 (s, 1H), 7.98 (m, 1H), 7.82 (s, 1H), 7.65 (d, 1H), 7.35 (d, 1H), 6.90 (m, 4H), |
| | 6.80-6.75 (m, 4H), 6.80 (m, 2H), 4.10 (m, 1H), 3.10 (m, 1H), 2.90 (m, 1H). |
| 30 | δ 8.95 (s, 1H), 8.30 (d, 1H), 8.10 (s, 1H), 7.80 (s, 1H), 7.73-7.65 (m, 2H), 7.60 |
| | (d, 1H), 7.43 (m, 2H), 7.35 (d, 1H), 7.20 (d, 1H), 4.10 (m, 1H), 3.10 (dd, 1H), 2.90- |
| | 2.80 (m, 1H). |
| 32 | δ 8.50 (s, 1H), 8.30 (s, 1H), 7.65 (d, 1H), 7.60 (d, 1H), 7.50 (s, 1H), 7.38-7.30 (m, |
| | 2H), 7.30 (m, 2H), 7.20 (s, 2H), 4.05 (m, 1H), 3.05-3.10 (m, 1H), 2.80-2.90 (m, 1H). |
| 33 | δ 8.65 (s, 1H), 8.4 (s, 1H), 8.35 (t, 1H), 7.8 (d, 1H), 7.67 (s, 1H), 7.40-7.30 (m, 2H), |
| | 7.25-7.15 (m, 5H), 4.05 (m, 1H), 3.05 (dd, 1H), 2.80-2.90 (m, 1H). |
| 38 | δ 8.65 (s, 1H), 8.40 (s, 1H), 7.55-7.65 (m, 5H), 7.50 (d, 1H), 7.50-7.40 (m, 2H), 7.30 |
| | (m, 1H), 7.20 (d, 1H), 6.90 (t, 1H), 4.15 (m, 1H), 3.10 (dd, 1H), 2.90 (m, 1H). |
| 40 | δ 8.65 (s, 2H), 7.85 (s, 1H), 7.75 (d, 2H), 7.70-7.60 (m, 2H), 7.40-7.30 (m, 6H), 4.10 |
| | (m, 1H), 3.10 (m, 1H), 2.90 (m, 1H). |
| 51 | δ 8.22 (s, 1H), 7.90 (s, 1H), 7.75 (d, 2H), 7.70-7.60 (m, 4H), 7.40 (d, 2H), 7.30 (m, |
| | 1H), 7.15 (d, 1H), 4.05 (m, 1H), 3.10-3.05 (d, 1H), 2.90-2.80 (m, 1H). |
| 56 | δ 9.18 (s, 1H), 8.50 (m, 2H), 8.00 (m, 1H), 7.95 (s, 1H), 7.80 (s, 1H), 7.65 (d, 1H), |
| | 7.34-7.40 (m, 2H), 7.25 (s, 1H), 7.20 (s, 1H), 4.15 (m, 1H), 3.10 (m, 1H), 2.90 |
| | (m, 1H). |
| 57 | δ 8.75 (s, 1H), 8.70 (s, 1H), 7.80 (s, 2H), 7.70 (m, 2H), 7.60 (d, 1H), 7.40 (d, 1H), |
| | 7.35 (s, 1H), 7.25, (m, 2H), 4.10 (m, 1H), 3.10 (d, 1H), 2.85-2.90 (m, 1H). |
| 58 | δ 8.70 (s, 2H), 7.85 (s, 1H), 7.80 (d, 1H), 7.70 (m, 1H), 7.63 (d, 1H), 7.52 (s, 1H), |
| | 7.47 (d, 1H), 7.35 (s, 1H), 7.30 (s, 1H), 7.25 (s, 1H), 4.15 (m, 1H), 3.05 (dd, 1H), |
| | 2.90 (m, 1H). |
| 69 | δ 9.10 (d, 1H), 8.90 (s, 1H), 8.10 (s, 1H), 7.90 (s, 1H), 7.70 (d, 2H), 7.45-7.38 |
| | (m, 3H), 7.20 (d, 1H), 4.10 (m, 1H), 3.10 (dd, 1H), 2.90-2.85 (m, 1H). |
| 102 | δ 7.88 (s, 1H), 7.62 (d, 2H), 7.57 (s, 7H), 7.52 (d, 1H), 4.22 (m, 1H), 3.20-3.16, (dd, |
| | 1H), 2.98-2.92 (dd, 1H) |
| 108 | δ 8.18 (s, 1H), 8.00 (s, 1H), 7.59-7.67 (m, 5H), 7.57 (d, 1H) 7.52, (d, 1H), 7.47 (m, |
| | 1H), 7.31 (d, 1H), 7.23, (d, 2H), 4.16 (m, 1H), 3.10-3.14 (dd, 1H), 2.91-2.86 (dd, 1H) |
| 109 | δ 8.35 (s, 1H), 8.16, (t, 1H), 8.01 (s, 1H), 7.72 (m, 2H), 7.60 (s, 2H), 7.55 (m, 1H), |
| | 7.50 (t, 1H), 7.37, (d, 1H), 4.19 (m, 1H), 3.16-3.11, (dd, 1H), 2.88-2.93 (dd, 1H) |

^{a1}H NMR spectra are reported in ppm relative to tetramethylsilane in CDCl₃ unless otherwise indicated.
Couplings are designated by (s)-singlet (d)-doublet, (t)-triplet, (q)-quartet, (dd)-doublet of doublets and (m)-multiplet.

INDEX TABLE E

| Cmp. No. | MS data (M + 1) |
|---|---|
| 122 | 545.23 |
| 125 | 529.27 |
| 145 | 565.39 |
| 149 | 569 |
| 150 | 549 |
| 154 | 533.37 |
| 155 | 517.41 |
| 156 | 533.29 |
| 157 | 517.25 |

### BIOLOGICAL EXAMPLES OF THE INVENTION

The following Tests demonstrate the control efficacy of compounds of this invention on specific pests. “Control efficacy” represents inhibition of invertebrate pest development (including mortality) that causes significantly reduced feeding. The pest control protection afforded by the compounds is not limited, however, to these species. See Index Tables A-B for compound descriptions.

### Formulation and Spray Methodology for Tests A-H

Test compounds were formulated using a solution containing 10% acetone, 90% water and 300 ppm Activator 90® non-ionic surfactant (Loveland Products, Loveland, Colo., USA). The formulated compounds were applied in 1 mL of liquid through an atomizer nozzle positioned 1.27 cm (0.5 inches) above the top of each test unit. Test compounds were sprayed at the rates indicated, and each test was replicated three times.

### Test A

For evaluating control of diamondback moth (Plutella xylostella (L.)) the test unit consisted of a small open container with a 12-14-day-old mustard plant inside. This was pre-infested with ˜50 neonate larvae that were dispensed into the test unit via corn cob grits using an inoculator. The larvae moved onto the test plant after being dispensed into the test unit.

Test compounds were formulated and sprayed at 250 and/or 50 ppm. After spraying of the formulated test compound, each test unit was allowed to dry for 1 hour and then a black, screened cap was placed on top. The test units were held for 6 days in a growth chamber at 25° C. and 70% relative humidity. Plant feeding damage was then visually assessed based on foliage consumed, and larvae were assessed for mortality.

Of the compounds of Formula 1 tested at 250 ppm, the following provided very good to excellent levels of control efficacy (40% or less feeding damage and/or 100% mortality): 1, 2, 3,4,5,6,7,8,9,10,11,12, 13,14, 15, 16,17, 19,20,21,22,23,24,25,26,27,28,29,30,32, 33, 34, 35, 36, 37, 38, 39, 40, 42, 43, 44, 46, 47, 48, 49, 50, 59, 61, 62, 63, 65, 66, 67, 68, 69, 70, 71, 72, 74, 76, 77, 90, and 123.

Of the compounds of Formula 1 tested at 50 ppm, the following provided very good to excellent levels of control efficacy (40% or less feeding damage and/or 100% mortality): 1, 2, 3, 4, 5, 6, 7, 8, 9, 10, 12, 13, 14, 16, 17, 19, 20, 21, 24, 25, 27, 29, 32, 33, 34, 35, 36, 38, 39, 41, 42, 44, 46, 47, 48, 49, 50, 52, 53, 54, 55, 59, 60, 61, 63, 65, 67, 68, 71, 72, 73, 74, 76, 78, 79, 80, 82, 83, 84, 85, 86, 88, 89, 90, 95, 96, 105, 113, 114, 115, 116, 117, 118, 120, 121, 122, 123, 124, 127, 128, 129, 130, 132, 133, 134, 135, 136, 138, 139, 140, 141, 149, 150, 151, 152, and 153.

Of the compounds of Formula 1 tested at 10 ppm, the following provided very good to excellent levels of control efficacy (40% or less feeding damage and/or 100% mortality): 3, 5, 6, 9, 10, 17, 24, 25, 39, 41, 52, 53, 54, 60, 61, 63, 68, 71, 76, 78, 79, 80, 82, 83, 85, 86, 89, 90, 96, 103, 105, 109, 113, 114, 115, 118, 123, 124, 126, 128, 129, 132, 133, 134, 138, 140, 141, 149, and 151.

### Test B

For evaluating control of fall armyworm (Spodoptera frugiperda (J. E. Smith)) the test unit consisted of a small open container with a 4-5-day-old corn (maize) plant inside. This was pre-infested with 10-15 1-day-old larvae on a piece of insect diet.

Test compounds were formulated and sprayed at 250 and/or 50 ppm. After spraying of the formulated test compound, the test units were maintained in a growth chamber for 6 days at 25° C. and 70% relative humidity. Plant feeding damage was then visually assessed based on foliage consumed, and larvae were assessed for mortality.

Of the compounds of Formula 1 tested at 250 ppm, the following provided very good to excellent levels of control efficacy (40% or less feeding damage and/or 100% mortality): 1, 2, 3,4,5,6,7,8,9,10,11,12, 13,14, 16, 17, 19,20,21,24,25,27,30,32,33,34,35,36,38,39, 42, 43, 49, 50, 51, 52, 53, 54, 55, 56, 60, 61, 63, 65, 67, 68, 71, 76, and 90.

Of the compounds of Formula 1 tested at 50 ppm, the following provided very good to excellent levels of control efficacy (40% or less feeding damage and/or 100% mortality): 1, 2, 3, 5, 6, 8, 9, 10, 13, 16, 17, 20, 24, 25, 27, 30, 32, 35, 39, 41, 52, 53, 54, 55, 60, 61, 63, 68, 71, 76, 78, 79, 80, 81, 82, 83, 84, 85, 88, 89, 90, 105, 113, 114, 115, 118, 123, 124, 128, 129, 132, 133, 134, 136, 138, 140, 141, 149, 152, and 153.

Of the compounds of Formula 1 tested at 10 ppm, the following provided very good to excellent levels of control efficacy (40% or less feeding damage and/or 100% mortality): 1, 5, 17, 39, 52, 60, 63, 68, 71, 76, 78, 79, 82, 83, 100, 103, 105, 113, 115, 118, 123, 124, 128, 138, and 153.

### Test C

For evaluating control of corn planthopper (Peregrinus maidis (Ashmead)) through contact and/or systemic means, the test unit consisted of a small open container with a 3-4-day-old corn (maize) plant inside. White sand was added to the top of the soil prior to application of the test compound.

Test compounds were formulated and sprayed at 250 ppm. After spraying of the formulated test compound, the test units were allowed to dry for 1 h before they were post-infested with -15-20 nymphs (18-to-21-day-old). A black, screened cap was placed on the top of each test unit, and the test units were held for 6 days in a growth chamber at 22-24° C. and 50-70% relative humidity. Each test unit was then visually assessed for insect mortality.

Of the compounds of Formula 1 tested at 250 ppm, no noticeable activity was observed.

### Test D

For evaluating control of potato leafhopper (Empoasca fabae (Harris)) through contact and/or systemic means, the test unit consisted of a small open container with a 5-6-day-old Soleil bean plant (primary leaves emerged) inside. White sand was added to the top of the soil, and one of the primary leaves was excised prior to application of the test compound.

Test compounds were formulated and sprayed at 250 and/or 50 ppm. After spraying of the formulated test compound, the test units were allowed to dry for 1 hour before they were post-infested with 5 potato leafhoppers (18-to-21-day-old adults). A black, screened cap was placed on the top of the test unit, and the test units were held for 6 days in a growth chamber at 20° C. and 70% relative humidity. Each test unit was then visually assessed for insect mortality.

Of the compounds of Formula 1 tested at 250 ppm, the following resulted in at least 80% mortality: 1, 5, 13, 21, 24, 52, 60, and 71.

Of the compounds of Formula 1 tested at 50 ppm, the following resulted in at least 80% mortality: 52, 60, 103, 111, 115, 118, and 138.

### Test E

For evaluating control of green peach aphid (Myzus persicae (Sulzer)) through contact and/or systemic means, the test unit consisted of a small open container with a 12-15-day-old radish plant inside. This was pre-infested by placing on a leaf of the test plant 30-40 aphids on a piece of leaf excised from a culture plant (cut-leaf method). The aphids moved onto the test plant as the leaf piece desiccated. After pre-infestation, the soil of the test unit was covered with a layer of sand.

Test compounds were formulated and sprayed at 250 and/or 50 ppm. After spraying of the formulated test compound, each test unit was allowed to dry for 1 hour and then a black, screened cap was placed on top. The test units were held for 6 days in a growth chamber at 19-21° C. and 50-70% relative humidity. Each test unit was then visually assessed for insect mortality.

Of the compounds of Formula 1 tested at 250 ppm, no noticeable activity was observed.

### Test F

For evaluating control of cotton melon aphid (Aphis gossypii (Glover)) through contact and/or systemic means, the test unit consisted of a small open container with a 5-day-old okra plant inside. This was pre-infested with 30-40 insects on a piece of leaf according to the cut-leaf method, and the soil of the test unit was covered with a layer of sand.

Test compounds were formulated and sprayed at 250 and/or 50 ppm. After spraying, the test units were maintained in a growth chamber for 6 days at 19° C. and 70% relative humidity. Each test unit was then visually assessed for insect mortality.

Of the compounds of Formula 1 tested at 250 ppm, the following resulted in at least 80% mortality: 9, 24, 52, and 71.

### Test G

For evaluating control of the sweetpotato whitefly (Bemisia tabaci (Gennadius)) through contact and/or systemic means, the test unit consisted of a small open container with a 12-14-day-old cotton plant inside. Prior to the spray application, both cotyledons were removed from the plant, leaving one true leaf for the assay. Adult whiteflies were allowed to lay eggs on the plant and then were removed from the test unit. Cotton plants infested with at least 15 eggs were submitted to the test for spraying.

Test compounds were formulated and sprayed at 250 and/or 50 ppm. After spraying, the test units were allowed to dry for 1 hour. The cylinders were then removed, and the units were taken to a growth chamber and held for 13 days at 28° C. and 50-70% relative humidity. Each test unit was then visually assessed for insect mortality.

Of the compounds of Formula 1 tested at 250 ppm, the following resulted in at least 70% mortality: 7, 69, and 94.

### Test H

For evaluating control of the Western Flower Thrips (Frankliniellla occidentalis (Pergande)) through contact and/or systemic means, the test unit consisted of a small open container with a 5-7-day-old Soleil bean plant inside.

Test compounds were formulated and sprayed at 250 and/or 50 ppm. After spraying, the test units were allowed to dry for 1 hour, and then about 60 thrips (adults and nymphs) were added to each unit. A black, screened cap was placed on top, and the test units were held for 6 days at 25° C. and 45-55% relative humidity. Each test unit was then visually assessed for plant damage and insect mortality.

Of the compounds of Formula 1 tested at 250 ppm, the following provided very good to excellent levels of control efficacy (30% or less plant damage and/or 100% mortality): 1, 21, 28, 30, 32, 33, 36, 37, 44, 52, 59, 60, 62, 67, 68, 71, 75, and 76.

Of the compounds of Formula 1 tested at 50 ppm, the following provided very good to excellent levels of control efficacy (30% or less plant damage and/or 100% mortality): 1, 21, 28, 30, 32, 37, 59, 60, 62, 67, 71, and 76.

What is claimed is:

1. A compound selected from Formula 1, an N-oxide or salt thereof, 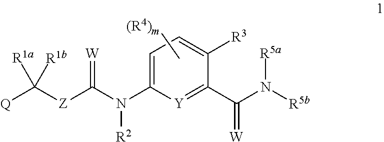
wherein
Q is a phenyl ring or a naphthalenyl ring system, each ring or ring system optionally substituted with up to 5 substituents independently selected from R⁶; or a 5- to 6-membered heterocyclic ring or an 8- to 11-membered heteroaromatic bicyclic ring system, each ring or ring system containing ring members selected from carbon atoms and 1 to 4 heteroatoms independently selected from up to 2 O, up to 2 S and up to 4 N atoms, wherein up to 2 ring members are independently selected from C(═O), C(═S), S(═O) and S(═O)₂, each ring or ring system optionally substituted with up to 5 substituents independently selected from R⁶;
Y is CR⁴ or N;
R^{1a} is CF₃, CHF₂, CCl₃, CHCl₂, CF₂Cl, CFCl₂ or CHFCl;
R^{1b} is H, halogen, hydroxy, C₁-C₃ alkyl, C₁-C₃ haloalkyl, C₁-C₃ alkoxy or C₁-C₃ haloalkoxy;
Z is CR^{7a}R^{7b}, NR^{7c}, O or S;
each W is independently O or S;
R² is H; or C₁-C₆ alkyl, C₂-C₆ alkenyl, C₂-C₆ alkynyl, C₂-C₆ alkylcarbonyl, C₂-C₆ alkoxycarbonyl or C₁-C₆ alkylsulfonyl, each optionally substituted with up to 5 substituents independently selected from halogen, cyano, hydroxy, C₁-C₃ alkyl, C₁-C₃ haloalkyl, C₁-C₃ alkoxy and C₁-C₃ haloalkoxy;
R³ is H, halogen, cyano, nitro, C₁-C₆ alkyl, C₁-C₆ haloalkyl, OR⁸ or S(═O)ₜR⁸;
each R⁴ is independently halogen, cyano, nitro, C₁-C₆ alkyl, C₁-C₆ haloalkyl, OR⁸ or S(═O)ₜR⁸;
m is 0, 1, 2, or 3;
each t is independently 0, 1 or 2;
R^{5a} is H, cyano, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₂-C₆ alkoxyalkyl, C₂-C₆ haloalkoxyalkyl, C₁-C₆ alkylsulfonyl, C₁-C₆ haloalkylsulfonyl, C₂-C₆ alkylthioalkyl, C₂-C₆ alkylsulfinylalkyl, C₂-C₆ alkylsulfonylalkyl, C₂-C₆ alkylcarbonyl, C₂-C₆ haloalkylcarbonyl, C₂-C₆ alkoxycarbonyl, C₂-C₆ haloalkoxycarbonyl, C₂-C₆ alkylaminocarbonyl, C₃-C₆ dialkylaminocarbonyl, C₃-C₆ alkylaminocarbonylalkyl or C₃-C₆ haloalkylaminocarbonylalkyl;
R^{5b} is H, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₁-C₆ hydroxyalkyl, C₂-C₆ cyanoalkyl, C₃-C₆ cycloalkyl, C₃-C₆ halocycloalkyl, C₃-C₆ cycloalkenyl, C₃-C₆ halocycloalkenyl, C₄-C₁₀ alkylcycloalkyl, C₄-C₁₀ haloalkylcycloalkyl, C₄-C₁₀ cycloalkylalkyl, C₄-C₁₀ halocycloalkylalkyl, C₅-C₁₀ alkylcycloalkylalkyl, C₂-C₆ alkoxyalkyl, C₂-C₆ haloalkoxyalkyl, C₄-C₁₀ cycloalkoxyalkyl, C₃-C₆ alkoxyalkoxyalkyl, C₂-C₆ alkylthioalkyl, C₂-C₆ haloalkylthioalkyl, C₂-C₆ alkylsulfinylalkyl, C₂-C₆ haloalkylsulfinylalkyl, C₂-C₆ alkylsulfonylalkyl, C₂-C₆ haloalkylsulfonylalkyl, C₂-C₆ alkylaminoalkyl, C₂-C₆ haloalkylaminoalkyl, C₃-C₈ dialkylaminoalkyl, C₄-C₁₀ cycloalkylaminoalkyl; or
R^{5b} is a phenyl ring or a naphthalenyl ring system, each ring or ring system optionally substituted with up to 5 substituents independently selected from R⁹; or a 5- to 6-membered heterocyclic ring or an 8- to 11-membered heteroaromatic bicyclic ring system, each ring or ring system containing ring members selected from carbon atoms and 1 to 4 heteroatoms independently selected from up to 2 O, up to 2 S and up to 4 N atoms, wherein up to 2 ring members are independently selected from C(═O), C(═S), S(═O) and S(═O)₂, each ring optionally substituted with up to 5 substituents independently selected from R⁹; or
R^{5b} is -A(CR^{10a}R^{10b})ₙB or NR^{21a}R^{21b}; or
R^{5a} and R^{5b} are taken together to form a 4- to 6-membered fully saturated heterocyclic ring, each ring containing ring members, in addition to the connecting nitrogen atom, selected from carbon atoms and up to 2 heteroatoms independently selected from up to 2 O, up to 2 S and up to 2 N atoms, each ring optionally substituted with up to 3 substituents independently selected from R¹³;
A is a direct bond, O or NR¹¹;
n is 0, 1, 2 or 3, provided that when n is 0, then A is O or NR¹¹;
B is a phenyl ring optionally substituted with up to 5 substituents independently selected from R¹²; or a 4- to 7-membered heterocyclic ring containing ring members selected from carbon atoms and 1 to 4 heteroatoms independently selected from up to 2 O, up to 2 S and up to 4 N atoms, wherein up to 3 carbon atom ring members are independently selected from C(═O) and C(═S), the ring optionally substituted with up to 3 substituents independently selected from R¹²;
each R⁶ is independently cyano, halogen, hydroxy, nitro, C(═O)OH, NR^{14a}R^{14b}, C(═O)NR^{14a}R^{14b}, C(═S)NR^{14a}R^{14b} or —U—V-T; or C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₃-C₆ cycloalkyl, C₃-C₆ halocycloalkyl, C₂-C₆ cyanoalkyl, C₁-C₆ hydroxyalkyl, C₄-C₁₀ alkylcycloalkyl, C₄-C₁₀ cycloalkylalkyl, C₃-C₆ cycloalkenyl, C₃-C₆ halocycloalkenyl, C₂-C₆ alkoxyalkyl, C₄-C₁₀ cycloalkoxyalkyl, C₃-C₁₀ alkoxyalkoxyalkyl, C₂-C₆ alkylthioalkyl, C₂-C₆ alkylsulfinylalkyl, C₁-C₆ alkoxy, C₁-C₆ haloalkoxy, C₃-C₆ cycloalkoxy, C₃-C₆ halocycloalkoxy, C₄-C₁₀ cycloalkylalkoxy, C₂-C₆ alkenyloxy, C₂-C₆ haloalkenyloxy, C₂-C₆ alkoxyalkoxy, C₂-C₆ alkylcarbonyloxy, C₁-C₆ alkylthio, C₁-C₆ haloalkylthio, C₃-C₆ cycloalkylthio, C₁-C₆ alkylsulfinyl, C₁-C₆ haloalkylsulfinyl, C₁-C₆ alkylsulfonyl, C₁-C₆ haloalkylsulfonyl, C₃-C₆ cycloalkylsulfonyl, C₁-C₆ alkylamino, C₂-C₆ dialkylamino, C₁-C₆ haloalkylamino, C₂-C₆ halodialkylamino or C₃-C₆ cycloalkylamino, each optionally substituted with up to 3 substituents independently selected from R¹⁵;
R^{7a} is H, C₁-C₆ alkyl, C₁-C₆ haloalkyl, C₃-C₆ cycloalkyl, C₄-C₆ cycloalkylalkyl and C₄-C₆ alkylcycloalkyl;
R^{7b} is H, C₁-C₆ alkyl, C₁-C₆ haloalkyl, C₂-C₆ cyanoalkyl, C₃-C₆ cycloalkyl, C₃-C₆ halocycloalkyl, C₄-C₆ alkylcycloalkyl, C₄-C₆ cycloalkylalkyl, C₄-C₆ halocycloalkylalkyl, C₂-C₆ alkoxyalkyl, C₂-C₆ haloalkoxyalkyl, C₄-C₆ cycloalkoxyalkyl, C₃-C₆ alkoxyalkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₂-C₆ haloalkylaminoalkyl or C₃-C₆ dialkylaminoalkyl, each optionally substituted with up to 1 substituent selected from cyano, hydroxy, nitro, C₂-C₄ alkylcarbonyl or C₂-C₄ alkoxycarbonyl; or phenyl optionally substituted with up to 3 substituents independently selected from R¹⁶;
R^{7c} is H, C(═O)H, C₁-C₄ alkyl, C₁-C₄ haloalkyl or C₂-C₄ alkylcarbonyl;
each R⁸ is independently H, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₃-C₆ cycloalkyl or C₃-C₆ halocycloalkyl; or phenyl or benzyl each ring optionally substituted with up to 4 substituents independently selected from R¹⁷;
each R⁹ is independently amino, cyano, halogen, hydroxy, nitro, C₁-C₆ alkyl, C₁-C₆ haloalkyl, C₃-C₆ cycloalkyl, C₃-C₆ halocycloalkyl, C₁-C₆ alkoxy, C₁-C₆ haloalkoxy, C₂-C₆ alkoxyalkoxy, C₁-C₆ alkylthio, C₁-C₆ alkylsulfinyl, C₁-C₆ alkylsulfonyl, C₁-C₆ haloalkylsulfonyl, C₂-C₆ alkylcarbonyl, C₂-C₆ haloalkylcarbonyl, C₂-C₆ alkoxycarbonyl, C₁-C₆ alkylamino, C₂-C₆ dialkylamino, C₂-C₆ alkylaminocarbonyl or C₃-C₆ dialkylaminocarbonyl;
each R^{10a} is independently H, halogen, cyano or C₁-C₄ alkyl;
each R^{10b} is independently H or C₁-C₄ alkyl;
R¹¹ is H, cyano, hydroxy, C₁-C₃ alkyl, C₁-C₃ haloalkyl, C₂-C₃ alkenyl, C₂-C₃ haloalkenyl, C₁-C₃ alkoxy, C₂-C₃ alkylcarbonyl or C₂-C₃ haloalkylcarbonyl;
each R¹² is independently halogen, cyano, C₁-C₃ alkyl, C₁-C₃ haloalkyl, C₁-C₃ alkoxy or C₁-C₃ haloalkoxy;
each R¹³ is independently halogen, cyano, hydroxy, nitro, C₁-C₆ alkyl, C₁-C₆ haloalkyl, C₂-C₆ alkenyl, C₂-C₆ haloalkenyl, C₂-C₆ alkynyl C₂-C₆ haloalkynyl, C₃-C₆ cycloalkyl, C₃-C₆ halocycloalkyl, C₂-C₆ alkoxyalkyl, C₂-C₆ haloalkoxyalkyl, C₁-C₆ alkoxy, C₁-C₆ haloalkoxy, C₃-C₆ cycloalkoxy, C₃-C₆ halocycloalkoxy, C₂-C₆ alkenyloxy, C₂-C₆ haloalkenyloxy, C₂-C₆ alkynyloxy, C₂-C₆ haloalkynyloxy, C₂-C₆ alkoxyalkoxy, C₂-C₆ haloalkoxyalkoxy, C₁-C₆ alkylthio, C₁-C₆ haloalkylthio, C₂-C₆ alkylcarbonyl, C₂-C₆ haloalkylcarbonyl, C₂-C₆ alkoxycarbonyl, C₂-C₆ haloalkoxycarbonyl, C₂-C₆ alkylaminocarbonyl, C₃-C₆ dialkylaminocarbonyl, C₂-C₆ alkylcarbonyloxy, C₂-C₆ haloalkylcarbonyloxy, C₁-C₆ alkylamino, C₂-C₆ dialkylamino, C₁-C₆ haloalkylamino or C₂-C₆ halodialkylamino;
each R^{14a} is independently H, cyano, hydroxy, C₁-C₄ alkyl, C₁-C₄ haloalkyl, C₂-C₄ alkenyl, C₂-C₄ haloalkenyl, C₂-C₄ alkynyl, C₂-C₄ haloalkynyl, C₁-C₄ alkoxy, C₁-C₄ haloalkoxy, C₂-C₄ alkoxyalkyl, C₁-C₄ alkylsulfonyl, C₁-C₄ haloalkylsulfonyl, C₂-C₄ alkylthioalkyl, C₂-C₄ alkylsulfinylalkyl, C₂-C₄ alkylsulfonylalkyl, C₂-C₄ alkylcarbonyl, C₂-C₄ haloalkylcarbonyl, C₄-C₆ cycloalkylcarbonyl, C₂-C₄ alkoxycarbonyl, C₃-C₅ alkoxycarbonylalkyl, C₂-C₄ alkylaminocarbonyl or C₃-C₅ dialkylaminocarbonyl;
each R^{14b} is independently H, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₁-C₆ hydroxyalkyl, C₂-C₆ cyanoalkyl, C₃-C₆ cycloalkyl, C₃-C₆ halocycloalkyl, C₃-C₆ cycloalkenyl, C₃-C₆ halocycloalkenyl, C₄-C₁₀ alkylcycloalkyl, C₄-C₁₀ cycloalkylalkyl, C₄-C₁₀ halocycloalkylalkyl, C₂-C₆ alkoxyalkyl, C₂-C₆ haloalkoxyalkyl, C₄-C₁₀ cycloalkoxyalkyl, C₃-C₆ alkoxyalkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₂-C₆ haloalkylaminoalkyl, C₃-C₆ dialkylaminoalkyl or C₄-C₁₀ cycloalkylaminoalkyl;
each R¹⁵ is independently amino, cyano, halogen, hydroxy, nitro, C₁-C₄ alkyl, C₁-C₄ haloalkyl, C₃-C₆ cycloalkyl, C₃-C₆ halocycloalkyl, C₁-C₄ alkoxy, C₁-C₄ haloalkoxy, C₂-C₄ alkoxyalkoxy, C₁-C₄ alkylthio, C₁-C₄ alkylsulfinyl, C₁-C₄ alkylsulfonyl, C₁-C₄ haloalkylsulfonyl, C₂-C₄ alkylcarbonyl, C₂-C₄ haloalkylcarbonyl, C₂-C₅ alkoxycarbonyl, C₁-C₄ alkylamino or C₂-C₅ dialkylamino;
each U is independently a direct bond, C(═O)O, C(═O)N(R¹⁸) or C(═S)N(R¹⁹), wherein the atom to the left is connected to Q, and the atom to the right is connected to V;
each V is independently a direct bond; or C₁-C₆ alkylene, C₂-C₆ alkenylene, C₃-C₆ alkynylene, each optionally substituted with up to 3 substituents independently selected from halogen, cyano, nitro, hydroxy, C₁-C₂ alkyl, C₁-C₂ haloalkyl, C₁-C₂ alkoxy and C₁-C₂ haloalkoxy;
each T is independently phenyl or phenoxy, each optionally substituted with up to 3 substituents independently selected from R²⁰; or
each T is independently a 5- to 6-membered heteroaromatic ring, each ring containing ring members selected from carbon atoms and 1 to 4 heteroatoms independently selected from up to 2 O, up to 2 S and up to 4 N atoms, each ring optionally substituted with up to 3 substituents independently selected from R²⁰; or
each T is independently a 3- to 7-membered nonaromatic heterocyclic ring containing ring members selected from carbon atoms and 1 to 4 heteroatoms independently selected from up to 2 O, up to 2 S and up to 4 N atoms, wherein up to 2 ring members are independently selected from C(═O), C(═S), S(═O) and S(═O)₂, each ring optionally substituted with up to 3 substituents independently selected from R²⁰;
each R¹⁶ is independently halogen, cyano, nitro, C₁-C₆ alkyl, C₁-C₆ haloalkyl, C₁-C₆ alkoxy, C₁-C₆ haloalkoxy, C₁-C₆ alkylthio, C₁-C₆ haloalkylthio C₁-C₆ alkylsulfinyl, C₁-C₆ haloalkylsulfinyl, C₁-C₆ alkylsulfonyl or C₁-C₆ haloalkylsulfonyl;
each R¹⁷ is independently halogen, cyano, nitro, C₁-C₆ alkyl, C₁-C₆ haloalkyl, C₁-C₆ alkoxy, C₁-C₆ haloalkoxy, C₁-C₆ alkylthio, C₁-C₆ haloalkylthio C₁-C₆ alkylsulfinyl, C₁-C₆ haloalkylsulfinyl, C₁-C₆ alkylsulfonyl or C₁-C₆ haloalkylsulfonyl;
each R¹⁸ and R¹⁹ is independently H, cyano, hydroxy, C₁-C₄ alkyl, C₁-C₄ haloalkyl, C₂-C₄ alkylcarbonyl, C₂-C₄ haloalkylcarbonyl, C₂-C₄ alkoxycarbonyl or C₂-C₄ haloalkoxycarbonyl;
each R²⁰ is independently halogen, cyano, hydroxy, nitro, C₁-C₄ alkyl, C₁-C₄ haloalkyl, C₂-C₄ alkenyl, C₁-C₄ alkoxy, C₂-C₄ alkylcarbonyl or C₂-C₄ alkoxycarbonyl;
R^{21a} is H, C₁-C₄ alkyl, C₁-C₄ haloalkyl or C₂-C₄ alkylcarbonyl; and
R^{21b} is H, cyano, C₁-C₅ alkyl, C₁-C₅ haloalkyl, C₁-C₆ cycloalkyl, C₁-C₆ halocycloalkyl, C₂-C₅ alkylcarbonyl, C₂-C₅ haloalkylcarbonyl, C₄-C₇ cycloalkylcarbonyl, C₂-C₅ alkoxycarbonyl, C₂-C₅ haloalkoxycarbonyl, C₃-C₅ alkoxycarbonylalkyl, C₂-C₅ alkylaminocarbonyl or C₃-C₅ dialkylaminocarbonyl; or
R^{21a} and R^{21b} are taken together to form a 5- to 6-membered fully saturated heterocyclic ring, each ring containing ring members, in addition to the connecting nitrogen atom, selected from carbon atoms and up to 2 heteroatoms independently selected from up to 2 O, up to 2 S and up to 2 N atoms, each ring optionally substituted with up to 5 substituents independently selected from halogen, cyano, nitro, hydroxy, C₁-C₃ alkyl, C₁-C₃ haloalkyl, C₁-C₃ alkoxy and C₁-C₃ haloalkoxy.

2. The compound of claim 1 wherein
Q is selected from: 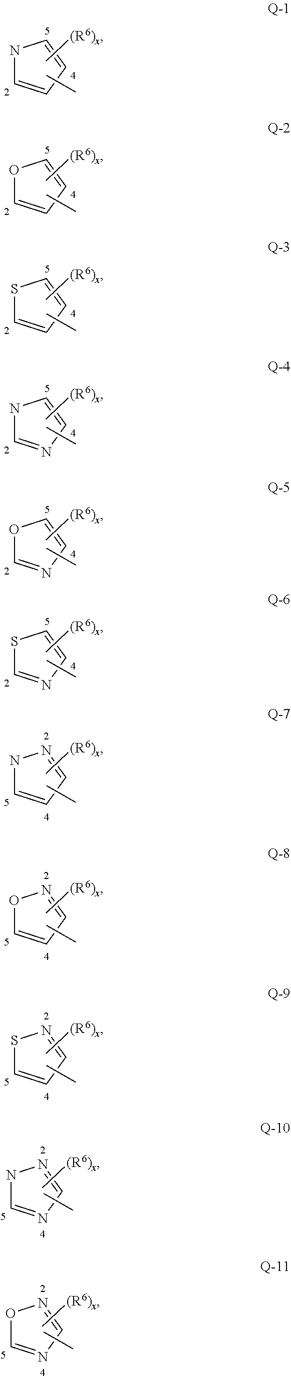 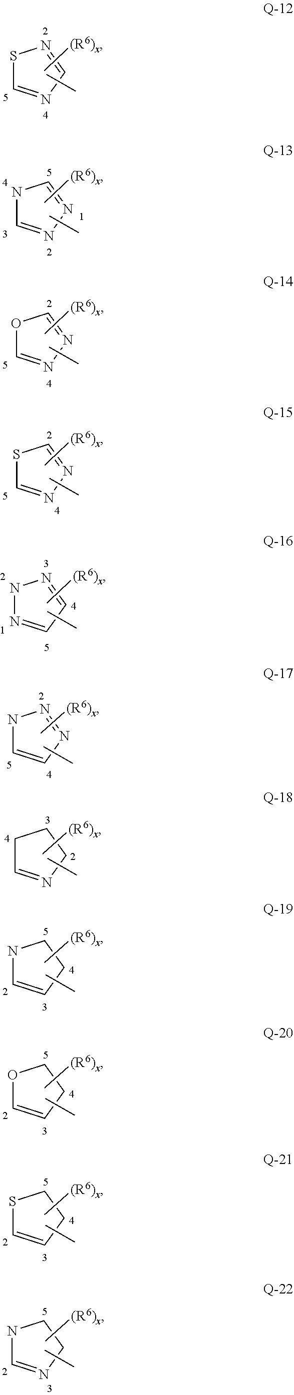 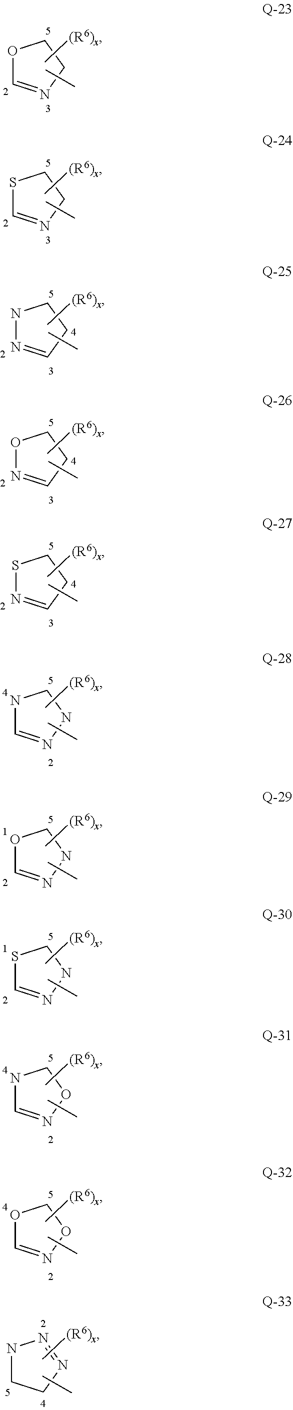 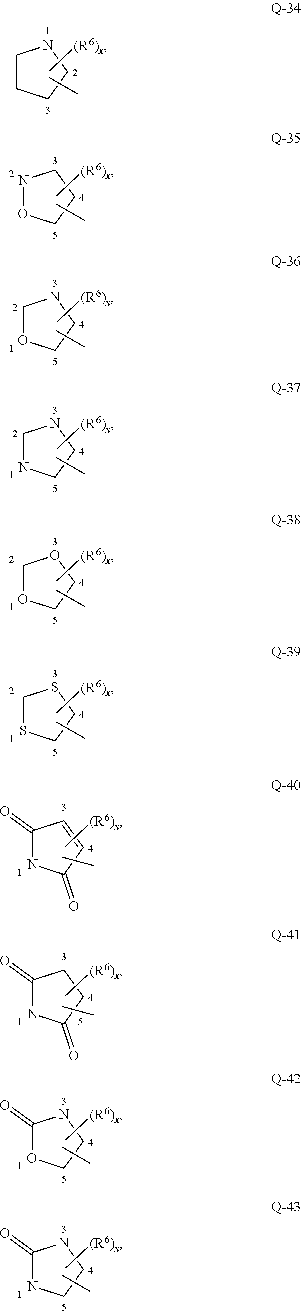 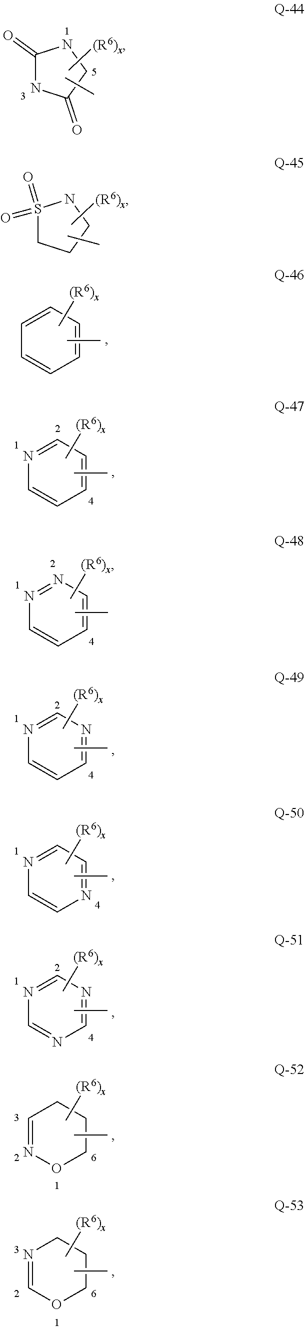 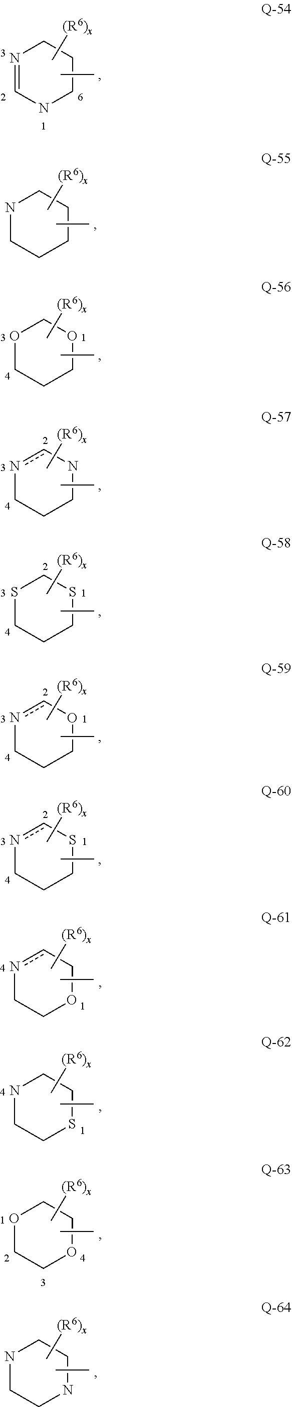 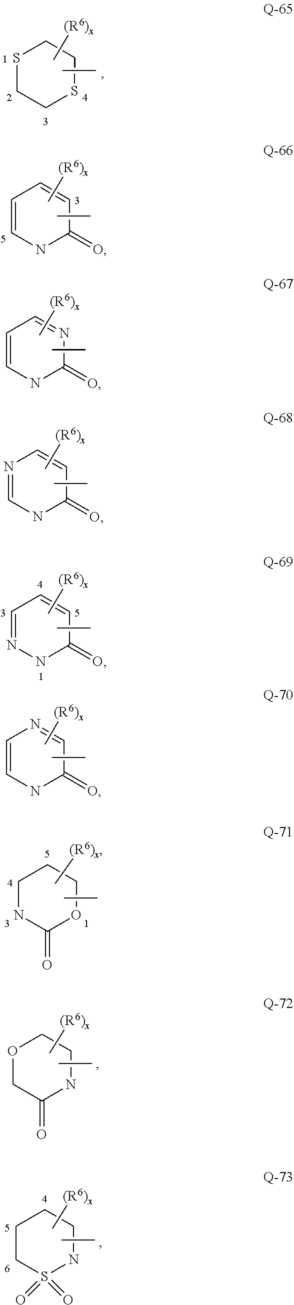 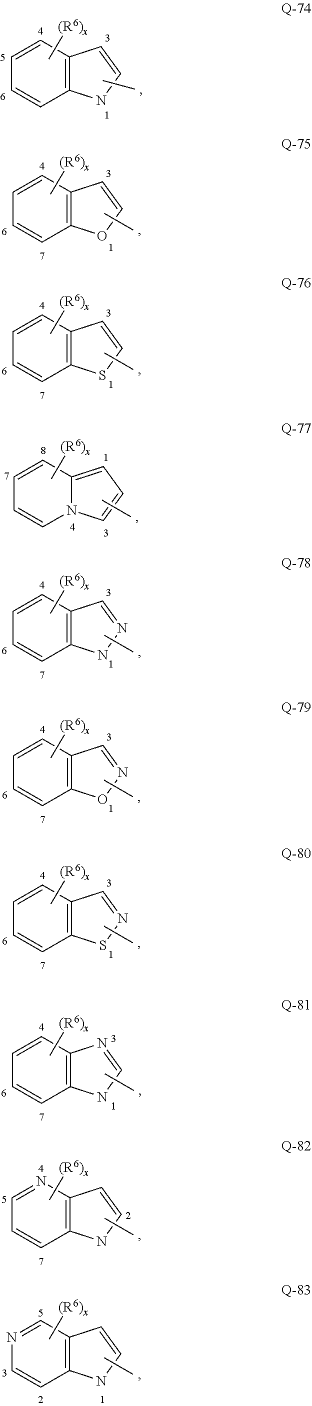 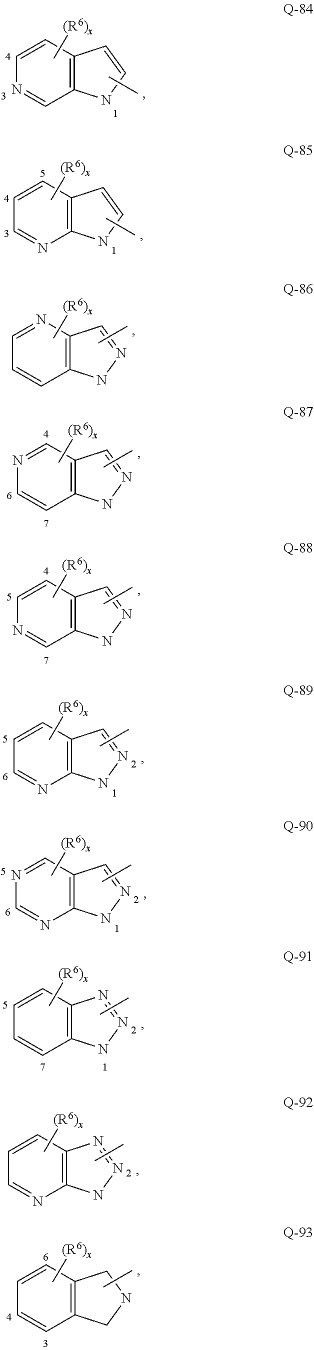 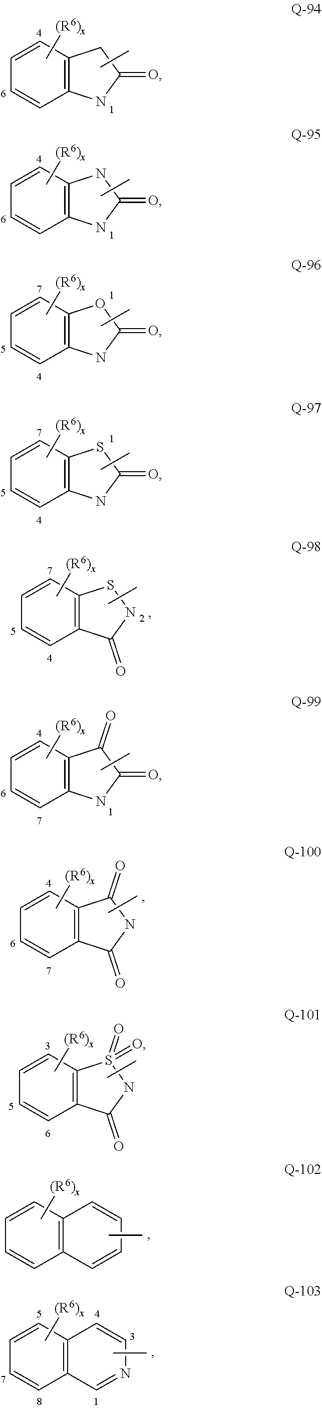 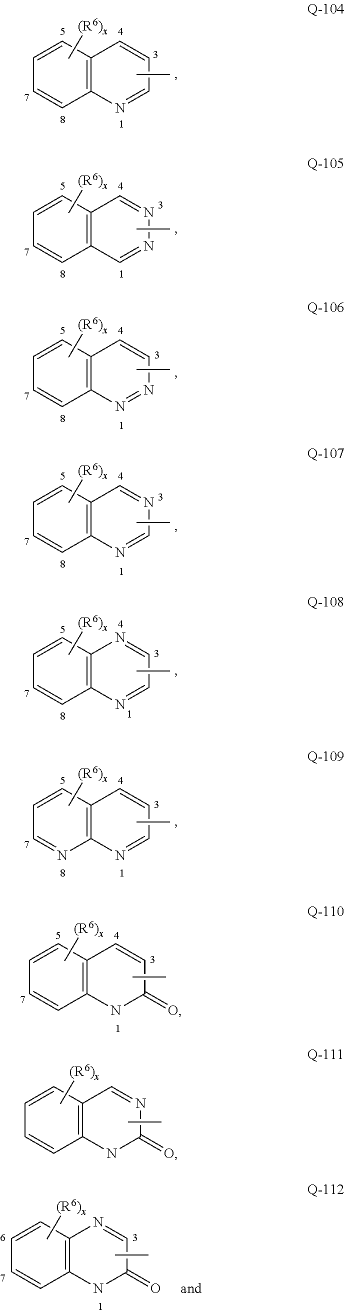 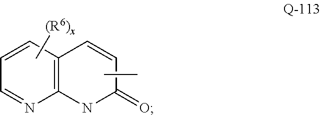
wherein the floating bond is connected to Formula 1 through any available carbon or nitrogen atom of the depicted ring or ring system; and x is 0, 1, 2 or 3;
R^{1a} is CF₃, CCl₃ or CF₂Cl;
R^{1b} is H, halogen, hydroxy, methyl, halomethyl, methoxy or halomethoxy;
Z is CR^{7a}R^{7b};
each W is O;
R² is H or methyl;
R³ is H, halogen, C₁-C₃ alkyl, C₁-C₃ haloalkyl or OR⁸;
R⁴ is halogen, cyano or C₁-C₂ alkyl;
m is 0 or 1;
R^{5a} is H, C₁-C₆ alkyl or C₁-C₆ haloalkyl;
R^{5b} is H, C₁-C₆ alkyl, C₁-C₆ haloalkyl, C₂-C₆ cyanoalkyl, C₃-C₆ cycloalkyl or C₄-C₁₀ cycloalkylalkyl; or a phenyl ring optionally substituted with up to 3 substituents independently selected from R⁹; or a 5- to 6-membered heterocyclic ring, each ring containing ring members selected from carbon atoms and 1 to 4 heteroatoms independently selected from up to 2 O, up to 2 S and up to 4 N atoms, wherein up to 2 ring members are independently selected from C(═O), C(═S), S(═O) and S(═O)₂, each ring optionally substituted with up to 3 substituents independently selected from R⁹; or -A(CR^{10a}R^{10b})ₙB or NR^{21a}R^{21b}; or
R^{5a} and R^{5b} are taken together to form a 5- to 6-membered fully saturated heterocyclic ring, each ring containing ring members, in addition to the connecting nitrogen atom, selected from carbon atoms and up to 2 heteroatoms independently selected from up to 2 O, up to 2 S and up to 2 N atoms, each ring optionally substituted with up to 3 substituents independently selected from R¹³;
A is O or direct bond;
n is 0 or 1;
B is a phenyl ring optionally substituted with up to 3 substituents independently selected from R¹²;
each R⁶ is independently cyano, halogen, nitro, NR^{14a}R^{14b}, C(═O)NR^{14a}R^{14b}, C(═S)NR^{14a}R^{14b} or —U—V-T; or C₁-C₆ alkyl, C₁-C₆ haloalkyl, C₂-C₆ alkenyl, C₂-C₆ haloalkenyl, C₃-C₆ cycloalkyl, C₃-C₆ halocycloalkyl, C₂-C₆ cyanoalkyl, C₄-C₁₀ alkylcycloalkyl, C₄-C₁₀ cycloalkylalkyl, C₂-C₆ alkoxyalkyl, C₂-C₆ alkylthioalkyl, C₁-C₆ alkoxy, C₁-C₆ haloalkoxy, C₂-C₆ alkenyloxy, C₂-C₆ haloalkenyloxy, C₂-C₆ alkoxyalkoxy, C₁-C₆ alkylthio, C₁-C₆ alkylamino, C₂-C₆ dialkylamino or C₁-C₆ haloalkylamino, each optionally substituted with up to 3 substituents independently selected from R¹⁵;
R^{7a} is H, C₁-C₃ alkyl or C₁-C₃ haloalkyl;
R^{7b} is H, C₁-C₃ alkyl, C₁-C₃ haloalkyl, C₃-C₆ cycloalkyl, C₃-C₆ halocycloalkyl, C₂-C₃ alkoxyalkyl; or phenyl optionally substituted with up to 3 substituents independently selected from R¹⁶;
R^{7c} is H, methyl, trifluoromethyl or C₂-C₄ alkylcarbonyl;
R⁸ is H, C₁-C₃ alkyl or C₁-C₃ haloalkyl; or phenyl optionally substituted with up to 3 substituents independently selected from R¹⁷;
each R⁹ is independently cyano, halogen, nitro, C₁-C₃ alkyl, C₁-C₃ haloalkyl, C₁-C₃ alkoxy, C₁-C₃ haloalkoxy, C₂-C₄ alkoxyalkoxy, C₁-C₃ alkylthio, C₂-C₄ alkylcarbonyl, C₂-C₄ alkoxycarbonyl, C₁-C₃ alkylamino or C₂-C₄ dialkylamino;
R^{10a} is H, Br, Cl, F, cyano or methyl;
R^{10b} is H or methyl;
each R¹² is independently halogen, methyl, halomethyl or methoxy;
each R¹³ is independently halogen, cyano, C₁-C₃ alkyl, C₁-C₃ haloalkyl, C₂-C₄ alkenyl, C₂-C₄ haloalkenyl, C₃-C₆ cycloalkyl, C₂-C₄ alkoxyalkyl, C₂-C₄ haloalkoxyalkyl, C₁-C₃ alkoxy, C₁-C₃ haloalkoxy, C₂-C₄ alkenyloxy, C₂-C₄ haloalkenyloxy, C₁-C₃ alkylthio, C₁-C₃ haloalkylthio, C₂-C₄ alkylcarbonyl, C₂-C₄ haloalkylcarbonyl;
each R^{14a} is independently H, C₁-C₂ alkyl, C₁-C₂ haloalkyl, C₂-C₄ alkoxyalkyl or C₂-C₄ alkylcarbonyl;
each R^{14b} is independently H, C₁-C₃ alkyl, C₁-C₃ haloalkyl, C₂-C₄ alkenyl, C₂-C₄ haloalkenyl, C₃-C₆ cycloalkyl, C₃-C₆ halocycloalkyl, C₄-C₁₀ cycloalkylalkyl, C₂-C₄ alkoxyalkyl or C₂-C₄ alkylaminoalkyl;
each R¹⁵ is independently, C₁-C₃ alkyl, C₁-C₃ haloalkyl, cyclopropyl, C₁-C₃ alkoxy, C₁-C₃ haloalkoxy, C₂-C₄ alkylcarbonyl or C₂-C₅ alkoxycarbonyl;
each U is independently direct bond, C(═O)O or C(═O)N(R¹⁸);
each V is independently a direct bond, C₁-C₃ alkylene, C₂-C₄ alkenylene or C₃-C₄ alkynylene;
each T is independently phenyl, pyridinyl or pyrazolyl, each optionally substituted with up to 2 substituents independently selected from R²⁰;
each R¹⁶ is independently halogen, C₁-C₃ alkyl, C₁-C₃ haloalkyl, C₁-C₃ alkoxy or C₁-C₃ haloalkoxy;
each R¹⁷ is independently halogen, cyano, C₁-C₃ alkyl, C₁-C₃ haloalkyl, C₁-C₃ alkoxy or C₁-C₃ haloalkoxy;
each R¹⁸ is independently H, cyano, hydroxy or C₁-C₂ alkyl;
each R²⁰ is independently halogen, cyano, C₁-C₂ alkyl, C₁-C₂ haloalkyl or C₁-C₂ alkoxy; and
R^{21a} is H, methyl, halomethyl or C₂-C₄ alkylcarbonyl;
R^{21b} is H, cyano, C₁-C₅ alkyl, C₁-C₅ haloalkyl, C₁-C₆ cycloalkyl, C₁-C₆ halocycloalkyl, C₂-C₅ alkylcarbonyl, C₂-C₅ haloalkylcarbonyl, C₂-C₅ alkoxycarbonyl, C₂-C₅ haloalkoxycarbonyl, C₃-C₅ alkoxycarbonylalkyl or C₂-C₅ alkylaminocarbonyl; or
R^{21a} and R^{21b} are taken together to form a 5- to 6-membered fully saturated heterocyclic ring, each ring containing ring members, in addition to the connecting nitrogen atom, selected from carbon atoms and up to 2 heteroatoms independently selected from up to 2 O, up to 2 S and up to 2 N atoms, each ring optionally substituted with up to 3 substituents independently selected from halogen, cyano, methyl, halomethyl or methoxy.

3. The compound of claim 2 wherein
Q is Q-46 through Q-50;
R^{1a} is CF₃;
R^{1b} is H, Br, Cl, F, hydroxy, methyl, halomethyl, methoxy or halomethoxy;
Z is CR^{7a}R^{7b};
R² is H; or C₁-C₃ alkyl, C₂-C₃ alkenyl, C₂-C₃ alkylcarbonyl or C₂-C₃ alkoxycarbonyl, each optionally substituted with up to 3 substituents independently selected from Br, Cl, F or methyl;
R³ is H or halogen;
R⁴ is Br, Cl, F or methyl;
R^{5a} is H, C₁-C₃ alkyl or C₁-C₃ haloalkyl;
R^{5b} is H, C₁-C₆ haloalkyl or C₃-C₆ cycloalkyl; or a phenyl ring optionally substituted with up to 3 substituents independently selected from R⁹; or a 6-membered heterocyclic ring, containing ring members selected from carbon atoms and 1 to 2 heteroatoms independently selected from up to 2 O, up to 2 S and up to 2 N atoms, each ring optionally substituted with up to 3 substituents independently selected from R⁹; or
R^{5a} and R^{5b} are taken together to form a 5- to 6-membered fully saturated heterocyclic ring, each ring containing ring members, in addition to the connecting nitrogen atom, selected from carbon atoms and up to 2 heteroatoms independently selected from up to 2 O, up to 2 S and up to 2 N atoms, each ring optionally substituted with up to 3 substituents independently selected from R¹³;
each R⁶ is halogen, C₁-C₃ alkyl, C₁-C₃ haloalkyl or C₁-C₃ alkoxy;
R^{7a} is H, methyl or trifluoromethyl;
R^{7b} is H, C₁-C₃ alkyl or C₁-C₃ haloalkyl;
each R⁹ is independently cyano, halogen, nitro, C₁-C₃ alkyl, C₁-C₃ haloalkyl, C₁-C₃ alkoxy or C₁-C₃ haloalkoxy; and
each R¹³ is independently halogen, C₁-C₃ alkyl, C₁-C₃ haloalkyl, C₂-C₄ alkenyl, C₂-C₄ haloalkenyl, C₁-C₃ alkoxy, C₁-C₃ haloalkoxy, C₁-C₃ alkylthio or C₁-C₃ haloalkylthio.

4. The compound of claim 3 wherein
Q is Q-46 or Q-47;
x is 1 or 2;
T is CR⁴ or N;
R^{1b} is H, Br, Cl, F, hydroxy or methyl;
R² is H or methyl;
R³ is halogen;
R⁴ is Br Cl or F;
R^{5a} is H or methyl;
R^{5b} is C₁-C₆ haloalkyl or C₁-C₆ cycloalkyl; or a phenyl or pyridinyl ring, each ring optionally substituted with up to 3 substituents independently selected from R⁹;
each R⁶ is independently Br, Cl, F or trifluoromethyl;
R^{7a} is H;
R^{7b} is H or methyl; and
each R⁹ is independently halogen, methyl, trifluoromethyl or methoxy.

5. The compound of claim 4 wherein
Q is Q-46;
T is CR⁴ or N;
R^{1b} is H;
R² is H;
m is 0;
R^{5a} is H;
R^{5b} is C₁-C₃ fluoroalkyl or cyclopropyl; or a phenyl or pyridinyl ring, each ring optionally substituted with up to 3 substituents independently selected from R⁹;
each R⁶ is independently Cl, F or trifluoromethyl;
R^{7b} is H; and
each R⁹ is independently Cl, F or trifluoromethyl.

6. The compound of claim 5 wherein
R^{5b} is cyclopropyl; or a phenyl, 2-pyridinyl or 3-pyridinyl ring, each ring optionally substituted with up to 3 substituents independently selected from R⁹.

7. The compound of claim 6 wherein
Q is Q-46 substituted at the 3- and 4-positions with substituents independently selected from R⁶; or Q is Q-46 substituted at the 3- and 5-positions with substituents independently selected from R⁶; or Q is Q-46 substituted at the 3-position with a substituent selected from R⁶; and
R^{5b} is a phenyl ring substituted at the 2-, 4- and 6-positions with substituents independently selected from R⁹; or a phenyl ring substituted at the 2- and 4-positions with substituents independently selected from R⁹; or a phenyl ring substituted at the 4-position with a substituent selected from R⁹.

8. A compound of claim 1 which is selected from the group:
3,4-dichloro-N-[4-chloro-3-[[(4-fluorophenyl)amino]carbonyl]phenyl]-β-(trifluoro-methyl)benzenepropanamide;
N-[4-chloro-3-[[(2,4-difluorophenyl)amino]carbonyl]phenyl]-β,3-bis(trifluoromethyl)-benzenepropanamide;
N-[4-chloro-3-[[[4-(trifluoromethyl)phenyl]amino]carbonyl]phenyl]-β,3-bis(trifluoro-methyl)benzenepropanamide;
3,5-dichloro-N-[4-chloro-3-[[(3-fluorophenyl)amino]carbonyl]phenyl]-β-(trifluoro-methyl)benzenepropanamide;
3,4-dichloro-N-[4-chloro-3-[[(2,4-difluorophenyl)amino]carbonyl]phenyl]-β-(trifluoro-methyl)benzenepropanamide;
3,5-dichloro-N-[4-chloro-3-[[(2,4-difluorophenyl)amino]carbonyl]phenyl]-β-(trifluoro-methyl)benzenepropanamide;
3,5-dichloro-N-[4-chloro-3-[[(4-fluorophenyl)amino]carbonyl]phenyl]-β-(trifluoro-methyl)benzenepropanamide;
N-[4-chloro-3-[[(2-chloro-4-fluorophenyl)amino]carbonyl]phenyl]-β,3-bis(trifluoromethyl)benzenepropanamide;
N-[4-chloro-3-[[(4-chloro-2-fluorophenyl)amino]carbonyl]phenyl]-β,3-bis(trifluoro-methyl)benzenepropanamide;
N-[4-chloro-3-[[(2,4,6-trifluorophenyl)amino]carbonyl]phenyl]-β,3-bis(trifluoromethyl)-benzenepropanamide.
3,5-dichloro-N-[4-chloro-3-[[(2,4-difluorophenyl)amino]carbonyl]phenyl]-4-fluoro-β-(trifluoromethyl)benzenepropanamide;
3-chloro-N-[4-chloro-3-[[(2,4-difluorophenyl)amino]carbonyl]phenyl]-4-fluoro-β-(trifluoromethyl)benzenepropanamide;
(βR)—N-[4-chloro-3-[[(2,4-difluorophenyl)amino]carbonyl]phenyl]-β,3-bis(trisfluoromethyl)benzenepropanamide;
N-[4-chloro-3-[[(2,4-difluorophenyl)amino]carbonyl]phenyl]-N-methyl-β,3-bis(trifluoromethyl)benzenepropanamide;
N-[4-chloro-3-[[(2,4-difluorophenyl)amino]carbonyl]phenyl]-β,3,5-tris(trifluoromethyl) benzenepropanamide; and
N-[4-chloro-3-[[(2,4-difluorophenyl)methylamino]carbonyl]phenyl]-β,3-bis(trifluoromethyl)benzenepropanamide;

9. A composition comprising a compound of claim 1 and at least one additional component selected from the group consisting of surfactants, solid diluents and liquid diluents, said composition optionally further comprising at least one additional biologically active compound or agent.

10. The composition of claim 9 wherein the at least one additional biologically active compound or agent is selected from the group consisting of abamectin, acephate, acequinocyl, acetamiprid, acrinathrin, afidopyropen, amidoflumet, amitraz, avermectin, azadirachtin, azinphos-methyl, benfuracarb, bensultap, bifenthrin, bifenazate, bistrifluron, borate, buprofezin, carbaryl, carbofuran, cartap, carzol, chlorantraniliprole, chlorfenapyr, chlorfluazuron, chlorpyrifos, chlorpyrifos-methyl, chromafenozide, clofentezin, clothianidin, cyantraniliprole, cyclaniliprole, cycloprothrin, cycloxaprid, cyflumetofen, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, diafenthiuron, diazinon, dieldrin, diflubenzuron, dimefluthrin, dimehypo, dimethoate, dinotefuran, diofenolan, emamectin, endosulfan, esfenvalerate, ethiprole, etofenprox, etoxazole, fenbutatin oxide, fenitrothion, fenothiocarb, fenoxycarb, fenpropathrin, fenvalerate, fipronil, flometoquin, flonicamid, flubendiamide, flucythrinate, flufenerim, flufenoxuron, flufenoxystrobin, flufensulfone, fluorpyram, flupiprole, flupyradifurone, fluvalinate, tau-fluvalinate, fonophos, formetanate, fosthiazate, halofenozide, heptafluthrin, hexaflumuron, hexythiazox, hydramethylnon, imidacloprid, indoxacarb, insecticidal soaps, isofenphos, lufenuron, malathion, meperfluthrin, metaflumizone, metaldehyde, methamidophos, methidathion, methiodicarb, methomyl, methoprene, methoxychlor, metofluthrin, monocrotophos, monofluthrin, methoxyfenozide, nitenpyram, nithiazine, novaluron, noviflumuron, oxamyl, parathion, parathion-methyl, permethrin, phorate, phosalone, phosmet, phosphamidon, pirimicarb, profenofos, profluthrin, propargite, protrifenbute, pyflubumide, pymetrozine, pyrafluprole, pyrethrin, pyridaben, pyridalyl, pyrifluquinazon, pyriminostrobin, pyriprole, pyriproxyfen, rotenone, ryanodine, silafluofen, spinetoram, spinosad, spirodiclofen, spiromesifen, spirotetramat, sulprofos, sulfoxaflor, tebufenozide, tebufenpyrad, teflubenzuron, tefluthrin, terbufos, tetrachlorvinphos, tetramethrin, tetramethylfluthrin, thiacloprid, thiamethoxam, thiodicarb, thiosultap-sodium, tolfenpyrad, tralomethrin, triazamate, trichlorfon, triflumuron, all strains of Bacillus thuringiensis, entomopathogenic bacteria, all strains of Nucleo polyhedrosis viruses, entomopathogenic viruses and entomopathogenic fungi.

11. The composition of claim 10 wherein the at least one additional biologically active compound or agent is selected from the group consisting of abamectin, acetamiprid, acrinathrin, afidopyropen, amitraz, avermectin, azadirachtin, benfuracarb, bensultap, bifenthrin, 3-bromo-1-(3-chloro-2-pyridinyl)-N-[4-cyano-2-methyl-6-[(methylamino)carbonyl]phenyl]-1H-pyrazole-5-carboxamide, buprofezin, carbaryl, cartap, chlorantraniliprole, chlorfenapyr, chlorpyrifos, clothianidin, cyantraniliprole, cyclaniliprole, cycloprothrin, cyfluthrin, beta-cyfluthrin, cyhalothrin, lambda-cyhalothrin, gamma-cyhalothrin, cypermethrin, alpha-cypermethrin, zeta-cypermethrin, cyromazine, deltamethrin, dieldrin, dinotefuran, diofenolan, emamectin, endosulfan, esfenvalerate, ethiprole, etofenprox, etoxazole, fenitrothion, fenothiocarb, fenoxycarb, fenvalerate, fipronil, flometoquin, flonicamid, flubendiamide, flufenoxuron, flufenoxystrobin, flufensulfone, flupiprole, flupyradifurone, fluvalinate, formetanate, fosthiazate, heptafluthrin, hexaflumuron, hydramethylnon, imidacloprid, indoxacarb, lufenuron, meperfluthirn, metaflumizone, methiodicarb, methomyl, methoprene, methoxyfenozide, metofluthrin, monofluthrin, nitenpyram, nithiazine, novaluron, oxamyl, pyflubumide, pymetrozine, pyrethrin, pyridaben, pyridalyl, pyriminostrobin, pyriproxyfen, ryanodine, spinetoram, spinosad, spirodiclofen, spiromesifen, spirotetramat, sulfoxaflor, tebufenozide, tetramethrin, thiacloprid, thiamethoxam, thiodicarb, thiosultap-sodium, tralomethrin, tetramethylfluthrin, triazamate, triflumuron, all strains of Bacillus thuringiensis and all strains of Nucleo polyhedrosis viruses.

12. A method for controlling an invertebrate pest comprising contacting the invertebrate pest or its environment with a biologically effective amount of a compound of any one of claims 1-8.
